SEQUENCE LISTING (720 sequences)

SEQ ID NO: 1 - RNA, 22 nt, Homo sapiens
cuccuacauauuagcauuaaca

SEQ ID NO: 2 - RNA, 22 nt, Homo sapiens
uccuguacugagcugccccgag

SEQ ID NO: 3 - RNA, 21 nt, Homo sapiens
aagccugcccggcuccucggg

SEQ ID NO: 4 - RNA, 22 nt, Homo sapiens
ccucccacacccaaggcuugca

SEQ ID NO: 5 - RNA, 20 nt, Homo sapiens
cuuccucgucugucugcccc

SEQ ID NO: 6 - RNA, 22 nt, Homo sapiens
caaucacuaacuccacugccau

SEQ ID NO: 7 - RNA, 21 nt, Homo sapiens
ucgaggagcucacagucuagu

SEQ ID NO: 8 - RNA, 23 nt, Homo sapiens
ucccccaggugugauucugauuu

SEQ ID NO: 9 - RNA, 22 nt, Homo sapiens
uucccuuugucauccuucgccu

SEQ ID NO: 10 - RNA, 23 nt, Homo sapiens
uacugcaucaggaacugauugga

SEQ ID NO: 11 - RNA, 22 nt, Homo sapiens
gccugcugggguggaaccuggu

SEQ ID NO: 12 - RNA, 21 nt, Homo sapiens
ucacuccucuccucccgucuu

SEQ ID NO: 13 - RNA, 21 nt, Homo sapiens
aaagugcuuccuuuuugaggg

SEQ ID NO: 14 - RNA, 23 nt, Homo sapiens
agguuacccgagcaacuuugcau

SEQ ID NO: 15 - RNA, 22 nt, Homo sapiens
caaagaauucuccuuuugggcu

SEQ ID NO: 16 - RNA, 22 nt, Homo sapiens
aaagugcaucuuuuuagaggau

SEQ ID NO: 17 - RNA, 23 nt, Homo sapiens
gcaaagcacacggccugcagaga

SEQ ID NO: 18 - RNA, 22 nt, Homo sapiens
ucgugucuuguguugcagccgg

SEQ ID NO: 19 - RNA, 23 nt, Homo sapiens
aucccuugcaggggcuguugggu

SEQ ID NO: 20 - RNA, 22 nt, Homo sapiens
ccgcacuguggguacuugcugc

SEQ ID NO: 21 - RNA, 21 nt, Homo sapiens
caaagaggaaggucccauuac

SEQ ID NO: 22 - RNA, 22 nt, Homo sapiens
uauagggauuggagccguggcg

SEQ ID NO: 23 - RNA, 22 nt, Homo sapiens
cuuucagucagauguuugcugc

SEQ ID NO: 24 - RNA, 21 nt, Homo sapiens
uccgguucucagggcuccacc

SEQ ID NO: 25 - RNA, 23 nt, Homo sapiens
cuggagauauggaagagcugugu

SEQ ID NO: 26 - RNA, 22 nt, Homo sapiens
aagcccuuaccccaaaaagcau

SEQ ID NO: 27 - RNA, 21 nt, Homo sapiens
guggcugcacucacuuccuuc

SEQ ID NO: 28 - RNA, 22 nt, Homo sapiens
ugucuacuacuggagacacugg

SEQ ID NO: 29 - RNA, 22 nt, Homo sapiens
uucuccaaaagggagcacuuuc

SEQ ID NO: 30 - RNA, 21 nt, Homo sapiens
gaaggcgcuucccuuuggagu

SEQ ID NO: 31 - RNA, 21 nt, Homo sapiens
aggcggagacuugggcaauug

SEQ ID NO: 32 - RNA, 22 nt, Homo sapiens
accuggcauacaauguagauuu

SEQ ID NO: 33 - RNA, 22 nt, Homo sapiens
acuuuaacauggaggcacuugc

SEQ ID NO: 34 - RNA, 21 nt, Homo sapiens
gcaguccaugggcauauacac

SEQ ID NO: 35 - RNA, 22 nt, Homo sapiens
aucaugaugggcuccucggugu

SEQ ID NO: 36 - RNA, 22 nt, Homo sapiens
ucuacagugcacgugucuccag

SEQ ID NO: 37 - RNA, 22 nt, Homo sapiens
aucgggaaugucguguccgccc

SEQ ID NO: 38 - RNA, 22 nt, Homo sapiens
uguaaacauccucgacuggaag

SEQ ID NO: 39 - RNA, 22 nt, Homo sapiens
aaagugcuucucuuuggugggu

SEQ ID NO: 40 - RNA, 23 nt, Homo sapiens
gcgaggaccccucggggucugac

SEQ ID NO: 41 - RNA, 21 nt, Homo sapiens
aauauaacacagauggccugu

SEQ ID NO: 42 - RNA, 22 nt, Homo sapiens
aaugcaccugggcaaggauuca

SEQ ID NO: 43 - RNA, 22 nt, Homo sapiens
cuccugagccauucugagccuc

SEQ ID NO: 44 - RNA, 21 nt, Homo sapiens
ccccaccuccucucuccucag

SEQ ID NO: 45 - RNA, 21 nt, Homo sapiens
gugucuuuugcucugcaguca

SEQ ID NO: 46 - RNA, 22 nt, Homo sapiens
ucagugcaucacagaacuuugu

SEQ ID NO: 47 - RNA, 22 nt, Homo sapiens
ucggauccgucugagcuuggcu

SEQ ID NO: 48 - RNA, 22 nt, Homo sapiens
gucauacacggcucuccucucu

SEQ ID NO: 49 - RNA, 21 nt, Homo sapiens
ccgucgccgccacccgagccg

SEQ ID NO: 50 - RNA, 21 nt, Homo sapiens
aaagcgcuucccuucagagug

SEQ ID NO: 51 - RNA, 22 nt, Homo sapiens
acugcauuaugagcacuuaaag

SEQ ID NO: 52 - RNA, 23 nt, Homo sapiens
aggaccugcgggacaagauucuu

SEQ ID NO: 53 - RNA, 22 nt, Homo sapiens
uaugucugcugaccaucaccuu

SEQ ID NO: 54 - RNA, 24 nt, Homo sapiens
acaaagugcuucccuuuagagugu

SEQ ID NO: 55 - RNA, 23 nt, Homo sapiens
caagucuuauuugagcaccuguu

SEQ ID NO: 56 - RNA, 23 nt, Homo sapiens
cgcauccccuagggcauuggugu

SEQ ID NO: 57 - RNA, 22 nt, Homo sapiens
aagcccuuaccccaaaaaguau

SEQ ID NO: 58 - RNA, 22 nt, Homo sapiens
aggcauugacuucucacuagcu

SEQ ID NO: 59 - RNA, 22 nt, Homo sapiens
auccgcgcucugacucucugcc

SEQ ID NO: 60 - RNA, 22 nt, Homo sapiens
agaucgaccguguuauauucgc

SEQ ID NO: 61 - RNA, 22 nt, Homo sapiens
caaagugccucccuuuagagug

SEQ ID NO: 62 - RNA, 23 nt, Homo sapiens
agcagcauuguacagggcuauga

SEQ ID NO: 63 - RNA, 22 nt, Homo sapiens
caagcucgugucuguggguccg

SEQ ID NO: 64 - RNA, 22 nt, Homo sapiens
cgggguuuugagggcgagauga

SEQ ID NO: 65 - RNA, 22 nt, Homo sapiens
uagcagcacauaaugguuugug

SEQ ID NO: 66 - RNA, 21 nt, Homo sapiens
gcgacccauacuugguuucag

SEQ ID NO: 67 - RNA, 25 nt, Homo sapiens
gcugggcagggcuucugagcuccuu

SEQ ID NO: 68 - RNA, 21 nt, Homo sapiens
uccuucugcuccgucccccag

SEQ ID NO: 69 - RNA, 21 nt, Homo sapiens
gaagugugccguggugugucu

SEQ ID NO: 70 - RNA, 21 nt, Homo sapiens
uggaggagaaggaaggugaug

SEQ ID NO: 71 - RNA, 22 nt, Homo sapiens
uaacugguugaacaacugaacc

SEQ ID NO: 72 - RNA, 22 nt, Homo sapiens
ugagguaguagguugugugguu

SEQ ID NO: 73 - RNA, 22 nt, Homo sapiens
aaagugcuucccuuuggacugu

SEQ ID NO: 74 - RNA, 19 nt, Homo sapiens
aggcugcggaauucaggac

SEQ ID NO: 75 - RNA, 23 nt, Homo sapiens
acuuacagacaagagccuugcuc

SEQ ID NO: 76 - RNA, 23 nt, Homo sapiens
uacuccagagggcgucacucaug

SEQ ID NO: 77 - RNA, 21 nt, Homo sapiens
uucacaguggcuaaguuccgc

SEQ ID NO: 78 - RNA, 22 nt, Homo sapiens
ugcuaugccaacauauugccau

SEQ ID NO: 79 - RNA, 22 nt, Homo sapiens
uguaacagcaacuccaugugga

SEQ ID NO: 80 - RNA, 20 nt, Homo sapiens
ccauggaucuccaggugggu

SEQ ID NO: 81 - RNA, 22 nt, Homo sapiens
caggauguggucaaguguuguu

SEQ ID NO: 82 - RNA, 19 nt, Homo sapiens
ugucucugcugggguuucu

SEQ ID NO: 83 - RNA, 23 nt, Homo sapiens
uaaggugcaucuagugcaguuag

SEQ ID NO: 84 - RNA, 22 nt, Homo sapiens
aggugguccguggcgcguucgc

SEQ ID NO: 85 - RNA, 22 nt, Homo sapiens
caauguuuccacagugcaucac

SEQ ID NO: 86 - RNA, 22 nt, Homo sapiens
aggggcuggcuuuccucugguc

SEQ ID NO: 87 - RNA, 17 nt, Homo sapiens
ucucgcuggggccucca

SEQ ID NO: 88 - RNA, 22 nt, Homo sapiens
ugcccuaaaugccccuucuggc

SEQ ID NO: 89 - RNA, 22 nt, Homo sapiens
uggagugugacaaugguguuug

SEQ ID NO: 90 - RNA, 21 nt, Homo sapiens
uugcucacuguucuucccuag

SEQ ID NO: 91 - RNA, 21 nt, Homo sapiens
cacuguguccuuucugcguag

SEQ ID NO: 92 - RNA, 21 nt, Homo sapiens
agggagggacgggggcugugc

SEQ ID NO: 93 - RNA, 21 nt, Homo sapiens
aaggcagggcccccgcucccc

SEQ ID NO: 94 - RNA, 22 nt, Homo sapiens
cuauacagucuacugucuuucc

SEQ ID NO: 95 - RNA, 22 nt, Homo sapiens
aaucauacacgguugaccuauu

SEQ ID NO: 96 - RNA, 24 nt, Homo sapiens
acugggggcuuucgggcucugcgu

SEQ ID NO: 97 - RNA, 21 nt, Homo sapiens
ugguucuagacuugccaacua

SEQ ID NO: 98 - RNA, 21 nt, Homo sapiens
cugaccuaugaauugacagcc

SEQ ID NO: 99 - RNA, 22 nt, Homo sapiens
cucuagagggaagcgcuuucug

SEQ ID NO: 100 - RNA, 20 nt, Homo sapiens
agagguauagggcaugggaa

SEQ ID NO: 101 - RNA, 21 nt, Homo sapiens
uuaagacuugcagugauguuu

SEQ ID NO: 102 - RNA, 22 nt, Homo sapiens
aaaaguaauugcggauuuugcc

SEQ ID NO: 103 - RNA, 23 nt, Homo sapiens
cugggaucuccggggucuugguu

SEQ ID NO: 104 - RNA, 22 nt, Homo sapiens
cuccuauaugaugccuuucuuc

SEQ ID NO: 105 - RNA, 22 nt, Homo sapiens
aaaaugguucccuuuagagugu

SEQ ID NO: 106 - RNA, 21 nt, Homo sapiens
cggggcagcucaguacaggau

SEQ ID NO: 107 - RNA, 23 nt, Homo sapiens
caaagugcuuacagugcagguag

SEQ ID NO: 108 - RNA, 22 nt, Homo sapiens
ugcaacuuaccugagucauuga

SEQ ID NO: 109 - RNA, 22 nt, Homo sapiens
accaucgaccguugauuguacc

SEQ ID NO: 110 - RNA, 22 nt, Homo sapiens
gaaggcgcuucccuuuagagcg

SEQ ID NO: 111 - RNA, 22 nt, Homo sapiens
cacacacugcaauuacuuuugc

SEQ ID NO: 112 - RNA, 21 nt, Homo sapiens
uuaauaucggacaaccauugu

SEQ ID NO: 113 - RNA, 22 nt, Homo sapiens
aacaauauccuggugcugagug

SEQ ID NO: 114 - RNA, 24 nt, Homo sapiens
agcagaagcagggagguucuccca

SEQ ID NO: 115 - RNA, 22 nt, Homo sapiens
agucauuggaggguuugagcag

SEQ ID NO: 116 - RNA, 22 nt, Homo sapiens
ccuguucuccauuacuuggcuc

SEQ ID NO: 117 - RNA, 25 nt, Homo sapiens
aaaggauucugcugucggucccacu

SEQ ID NO: 118 - RNA, 22 nt, Homo sapiens
cacuagauugugagcuccugga

SEQ ID NO: 119 - RNA, 24 nt, Homo sapiens
gaccuggacauguuugugcccagu

SEQ ID NO: 120 - RNA, 22 nt, Homo sapiens
ucagugcacuacagaacuuugu

SEQ ID NO: 121 - RNA, 22 nt, Homo sapiens
acgcccuucccccccuucuuca

SEQ ID NO: 122 - RNA, 21 nt, Homo sapiens
ucguggccuggucuccauuau

SEQ ID NO: 123 - RNA, 22 nt, Homo sapiens
agagguaguagguugcauaguu

SEQ ID NO: 124 - RNA, 22 nt, Homo sapiens
uaauacugucugguaaaaccgu

SEQ ID NO: 125 - RNA, 23 nt, Homo sapiens
agguuguccguggugaguucgca

SEQ ID NO: 126 - RNA, 22 nt, Homo sapiens
ccaauauuggcugugcugcucc

SEQ ID NO: 127 - RNA, 22 nt, Homo sapiens
uaacagucuacagccauggucg

SEQ ID NO: 128 - RNA, 23 nt, Homo sapiens
uauggcuuuucauuccuauguga

SEQ ID NO: 129 - RNA, 22 nt, Homo sapiens
uauugcacauuacuaaguugca

SEQ ID NO: 130 - RNA, 22 nt, Homo sapiens
ugaccgauuucuccugguguuc

SEQ ID NO: 131 - RNA, 22 nt, Homo sapiens
aacccguagauccgaacuugug

SEQ ID NO: 132 - RNA, 23 nt, Homo sapiens
cacucagccuugagggcacuuuc

SEQ ID NO: 133 - RNA, 22 nt, Homo sapiens
cuacaaagggaagcacuuucuc

SEQ ID NO: 134 - RNA, 22 nt, Homo sapiens
aggcagcgggguguaguggaua

SEQ ID NO: 135 - RNA, 23 nt, Homo sapiens
aaagugcugcgacauuugagcgu

SEQ ID NO: 136 - RNA, 20 nt, Homo sapiens
cugcaaagggaagcccuuuc

SEQ ID NO: 137 - RNA, 21 nt, Homo sapiens
agaggauacccuuuguauguu

SEQ ID NO: 138 - RNA, 21 nt, Homo sapiens
gaaagcgcuucucuuuagagg

SEQ ID NO: 139 - RNA, 22 nt, Homo sapiens
gugagucucuaagaaaagagga

SEQ ID NO: 140 - RNA, 22 nt, Homo sapiens
accacugaccguugacuguacc

SEQ ID NO: 141 - RNA, 20 nt, Homo sapiens
ucugcaggguuugcuuugag

SEQ ID NO: 142 - RNA, 22 nt, Homo sapiens
caucuuacugggcagcauugga

SEQ ID NO: 143 - RNA, 19 nt, Homo sapiens
ucuaggcugguacugcuga

SEQ ID NO: 144 - RNA, 22 nt, Homo sapiens
aaaccuguguuguucaagaguc

SEQ ID NO: 145 - RNA, 21 nt, Homo sapiens
uguucauguagauguuuaagc

SEQ ID NO: 146 - RNA, 22 nt, Homo sapiens
cggaugagcaaagaaagugguu

SEQ ID NO: 147 - RNA, 22 nt, Homo sapiens
aacacaccugguuaaccucuuu

SEQ ID NO: 148 - RNA, 23 nt, Homo sapiens
uuucaagccagggggcguuuuuc

SEQ ID NO: 149 - RNA, 23 nt, Homo sapiens
ucaagagcaauaacgaaaaaugu

SEQ ID NO: 150 - RNA, 23 nt, Homo sapiens
cccaguguuuagacuaucuguuc

SEQ ID NO: 151 - RNA, 23 nt, Homo sapiens
ucccuguccuccaggagcucacg

SEQ ID NO: 152 - RNA, 22 nt, Homo sapiens
aaaagcuggguugagagggcga

SEQ ID NO: 153 - RNA, 23 nt, Homo sapiens
aacauucauuguugucggugggu

SEQ ID NO: 154 - RNA, 21 nt, Homo sapiens
gccccugggccuauccuagaa

SEQ ID NO: 155 - RNA, 23 nt, Homo sapiens
uaagugcuuccauguuuugguga

SEQ ID NO: 156 - RNA, 22 nt, Homo sapiens
aaaaguacuugcggauuuugcu

SEQ ID NO: 157 - RNA, 20 nt, Homo sapiens
agagucuugugaugucuugc

SEQ ID NO: 158 - RNA, 23 nt, Homo sapiens
ugagcgccucgacgacagagccg

SEQ ID NO: 159 - RNA, 22 nt, Homo sapiens
cugaagcucagagggcucugau

SEQ ID NO: 160 - RNA, 22 nt, Homo sapiens
uuugguccccuucaaccagcua

SEQ ID NO: 161 - RNA, 21 nt, Homo sapiens
ccacaccguaucugacacuuu

SEQ ID NO: 162 - RNA, 22 nt, Homo sapiens
acuggacuuagggucagaaggc

SEQ ID NO: 163 - RNA, 23 nt, Homo sapiens
aguauguucuuccaggacagaac

SEQ ID NO: 164 - RNA, 22 nt, Homo sapiens
uuguacaugguaggcuuucauu

SEQ ID NO: 165 - RNA, 22 nt, Homo sapiens
uaaucucagcuggcaacuguga

SEQ ID NO: 166 - RNA, 22 nt, Homo sapiens
ugagaaccacgucugcucugag

SEQ ID NO: 167 - RNA, 22 nt, Homo sapiens
gaaguuguucgugguggauucg

SEQ ID NO: 168 - RNA, 21 nt, Homo sapiens
uaacagucuccagucacggcc

SEQ ID NO: 169 - RNA, 21 nt, Homo sapiens
uucaaguaauucaggauaggu

SEQ ID NO: 170 - RNA, 22 nt, Homo sapiens
cggguggaucacgaugcaauuu

SEQ ID NO: 171 - RNA, 22 nt, Homo sapiens
augguacccuggcauacugagu

SEQ ID NO: 172 - RNA, 21 nt, Homo sapiens
gcaggaacuugugagucuccu

SEQ ID NO: 173 - RNA, 27 nt, Homo sapiens
cacuguaggugauggugagagugggca

SEQ ID NO: 174 - RNA, 22 nt, Homo sapiens
aucgugcauccuuuuagagugu

SEQ ID NO: 175 - RNA, 22 nt, Homo sapiens
aaugcacccgggcaaggauucu

SEQ ID NO: 176 - RNA, 21 nt, Homo sapiens
acuggacuuggagucagaagg

SEQ ID NO: 177 - RNA, 21 nt, Homo sapiens
ucccacguuguggcccagcag

SEQ ID NO: 178 - RNA, 21 nt, Homo sapiens
aacaggugacugguuagacaa

SEQ ID NO: 179 - RNA, 22 nt, Homo sapiens
ugugacugguugaccagagggg

SEQ ID NO: 180 - RNA, 20 nt, Homo sapiens
agaccauggguucucauugu

SEQ ID NO: 181 - RNA, 22 nt, Homo sapiens
ccuauucuugguuacuugcacg

SEQ ID NO: 182 - RNA, 22 nt, Homo sapiens
acaguagagggaggaaucgcag

SEQ ID NO: 183 - RNA, 21 nt, Homo sapiens
uagcagcacagaaauauuggc

SEQ ID NO: 184 - RNA, 22 nt, Homo sapiens
ugccuacugagcugaaacacag

SEQ ID NO: 185 - RNA, 22 nt, Homo sapiens
aaaguucugagacacuccgacu

SEQ ID NO: 186 - RNA, 22 nt, Homo sapiens
uuuugcaauauguuccugaaua

SEQ ID NO: 187 - RNA, 21 nt, Homo sapiens
ugagaugaagcacuguagcuc

SEQ ID NO: 188 - RNA, 22 nt, Homo sapiens
ggauuccuggaaauacuguucu

SEQ ID NO: 189 - RNA, 22 nt, Homo sapiens
acggauguuugagcaugugcua

SEQ ID NO: 190 - RNA, 22 nt, Homo sapiens
uuuaacauggggguaccugcug

SEQ ID NO: 191 - RNA, 22 nt, Homo sapiens
aagauguggaaaaauuggaauc

SEQ ID NO: 192 - RNA, 22 nt, Homo sapiens
gcugcgcuuggauuucgucccc

SEQ ID NO: 193 - RNA, 23 nt, Homo sapiens
uccaguaccacgugucagggcca

SEQ ID NO: 194 - RNA, 23 nt, Homo sapiens
ucggggaucaucaugucacgaga

SEQ ID NO: 195 - RNA, 22 nt, Homo sapiens
cuugguucagggagggucccca

SEQ ID NO: 196 - RNA, 20 nt, Homo sapiens
cgugccacccuuuuccccag

SEQ ID NO: 197 - RNA, 22 nt, Homo sapiens
cucaucugcaaagaaguaagug

SEQ ID NO: 198 - RNA, 22 nt, Homo sapiens
cuuaugcaagauucccuucuac

SEQ ID NO: 199 - RNA, 22 nt, Homo sapiens
ugguugaccauagaacaugcgc

SEQ ID NO: 200 - RNA, 22 nt, Homo sapiens
ccaguggggcugcuguuaucug

SEQ ID NO: 201 - RNA, 22 nt, Homo sapiens
uaugcauuguauuuuuaggucc

SEQ ID NO: 202 - RNA, 23 nt, Homo sapiens
ugucacucggcucggcccacuac

SEQ ID NO: 203 - RNA, 23 nt, Homo sapiens
uaaggugcaucuagugcagauag

SEQ ID NO: 204 - RNA, 22 nt, Homo sapiens
cugguuucacaugguggcuuag

SEQ ID NO: 205 - RNA, 22 nt, Homo sapiens
cuauacaaccuacugccuuccc

SEQ ID NO: 206 - RNA, 22 nt, Homo sapiens
guucucccaacguaagcccagc

SEQ ID NO: 207 - RNA, 22 nt, Homo sapiens
aacuggaucaauuauaggagug

SEQ ID NO: 208 - RNA, 22 nt, Homo sapiens
ugugcgcagggagaccucuccc

SEQ ID NO: 209 - RNA, 22 nt, Homo sapiens
aaccaucgaccguugaguggac

SEQ ID NO: 210 - RNA, 22 nt, Homo sapiens
cgucaacacuugcugguuuccu

SEQ ID NO: 211 - RNA, 20 nt, Homo sapiens
aaaguagcuguaccauuugc

SEQ ID NO: 212 - RNA, 24 nt, Homo sapiens
cugaagugauguguaacugaucag

SEQ ID NO: 213 - RNA, 21 nt, Homo sapiens
cuauacaaucuacugucuuuc

SEQ ID NO: 214 - RNA, 22 nt, Homo sapiens
aucauagaggaaaauccauguu

SEQ ID NO: 215 - RNA, 22 nt, Homo sapiens
agagcuuagcugauuggugaac

SEQ ID NO: 216 - RNA, 22 nt, Homo sapiens
ugcaacgaaccugagccacuga

SEQ ID NO: 217 - RNA, 22 nt, Homo sapiens
caugccuugaguguaggaccgu

SEQ ID NO: 218 - RNA, 22 nt, Homo sapiens
gagcuuauucauaaaagugcag

SEQ ID NO: 219 - RNA, 23 nt, Homo sapiens
uaagugcuuccauguuuuaguag

SEQ ID NO: 220 - RNA, 24 nt, Homo sapiens
ucagaacaaaugccgguucccaga

SEQ ID NO: 221 - RNA, 19 nt, Homo sapiens
ugagcugcuguaccaaaau

SEQ ID NO: 222 - RNA, 22 nt, Homo sapiens
aacuggcccucaaagucccgcu

SEQ ID NO: 223 - RNA, 22 nt, Homo sapiens
ucguaccgugaguaauaaugcg

SEQ ID NO: 224 - RNA, 22 nt, Homo sapiens
aaccagcaccccaacuuuggac

SEQ ID NO: 225 - RNA, 21 nt, Homo sapiens
aagugaucuaaaggccuacau

SEQ ID NO: 226 - RNA, 22 nt, Homo sapiens
uagcuuaucagacugauguuga

SEQ ID NO: 227 - RNA, 21 nt, Homo sapiens
ccuggaaacacugagguugug

SEQ ID NO: 228 - RNA, 22 nt, Homo sapiens
auauuaccauuagcucaucuuu

SEQ ID NO: 229 - RNA, 21 nt, Homo sapiens
aggaggcagcgcucucaggac

SEQ ID NO: 230 - RNA, 25 nt, Homo sapiens
agggaucgcgggcggguggcggccu

SEQ ID NO: 231 - RNA, 22 nt, Homo sapiens
gaaagcgcuucccuuugcugga

SEQ ID NO: 232 - RNA, 21 nt, Homo sapiens
aggcaagaugcuggcauagcu

SEQ ID NO: 233 - RNA, 21 nt, Homo sapiens
aguuaggauuaggucguggaa

SEQ ID NO: 234 - RNA, 23 nt, Homo sapiens
ugcaccaugguugucugagcaug

SEQ ID NO: 235 - RNA, 21 nt, Homo sapiens
caucccuugcaugguggaggg

SEQ ID NO: 236 - RNA, 22 nt, Homo sapiens
gaugagcucauuguaauaugag

SEQ ID NO: 237 - RNA, 22 nt, Homo sapiens
uuaucagaaucuccagggguac

SEQ ID NO: 238 - RNA, 26 nt, Homo sapiens
gaugaugauggcagcaaauucugaaa

SEQ ID NO: 239 - RNA, 22 nt, Homo sapiens
uagcagcacaucaugguuuaca

SEQ ID NO: 240 - RNA, 26 nt, Homo sapiens
aaguaguugguuuguaugagaugguu

SEQ ID NO: 241 - RNA, 23 nt, Homo sapiens
ucugcucauaccccaugguuucu

SEQ ID NO: 242 - RNA, 22 nt, Homo sapiens
cugcgcaagcuacugccuugcu

SEQ ID NO: 243 - RNA, 20 nt, Homo sapiens
ggggagcuguggaagcagua

SEQ ID NO: 244 - RNA, 21 nt, Homo sapiens
uuuccauaggugaugagucac

SEQ ID NO: 245 - RNA, 22 nt, Homo sapiens
uccgucucaguuacuuuauagc

SEQ ID NO: 246 - RNA, 22 nt, Homo sapiens
uauaccucaguuuuaucaggug

SEQ ID NO: 247 - RNA, 21 nt, Homo sapiens
uucacaguggcuaaguucugc

SEQ ID NO: 248 - RNA, 27 nt, Homo sapiens
accuucuuguauaagcacugugcuaaa

SEQ ID NO: 249 - RNA, 23 nt, Homo sapiens
uuacaguuguucaaccaguuacu

SEQ ID NO: 250 - RNA, 22 nt, Homo sapiens
aguucuucaguggcaagcuuua

SEQ ID NO: 251 - RNA, 22 nt, Homo sapiens
ugucaguuugucaaauacccca

SEQ ID NO: 252 - RNA, 22 nt, Homo sapiens
aaaaguaauugcgguuuuugcc

SEQ ID NO: 253 - RNA, 22 nt, Homo sapiens
uauugcacuugucccggccugu

SEQ ID NO: 254 - RNA, 23 nt, Homo sapiens
cucuugagggaagcacuuucugu

SEQ ID NO: 255 - RNA, 22 nt, Homo sapiens
uggcucaguucagcaggaacag

SEQ ID NO: 256 - RNA, 24 nt, Homo sapiens
gcugguuucauauggugguuuaga

SEQ ID NO: 257 - RNA, 22 nt, Homo sapiens
gaaagugcuuccuuuuagaggc

SEQ ID NO: 258 - RNA, 21 nt, Homo sapiens
uccucuucucccuccucccag

SEQ ID NO: 259 - RNA, 24 nt, Homo sapiens
uuuggcaaugguagaacucacacu

SEQ ID NO: 260 - RNA, 22 nt, Homo sapiens
uuugguccccuucaaccagcug

SEQ ID NO: 261 - RNA, 22 nt, Homo sapiens
cguguucacagcggaccuugau

SEQ ID NO: 262 - RNA, 22 nt, Homo sapiens
ccucuuccccuugucucuccag

SEQ ID NO: 263 - RNA, 21 nt, Homo sapiens
cuucuugugcucuaggauugu

SEQ ID NO: 264 - RNA, 22 nt, Homo sapiens
ugagaccucuggguucugagcu

SEQ ID NO: 265 - RNA, 20 nt, Homo sapiens
guugugucaguuuaucaaac

SEQ ID NO: 266 - RNA, 22 nt, Homo sapiens
cugccaauuccauaggucacag

SEQ ID NO: 267 - RNA, 23 nt, Homo sapiens
gaacgccuguucuugccaggugg

SEQ ID NO: 268 - RNA, 22 nt, Homo sapiens
acuuguaugcuagcucagguag

SEQ ID NO: 269 - RNA, 22 nt, Homo sapiens
uggugggcacagaaucuggacu

SEQ ID NO: 270 - RNA, 22 nt, Homo sapiens
ggguggggauuuguugcauuac

SEQ ID NO: 271 - RNA, 21 nt, Homo sapiens
cacauuacacggucgaccucu

SEQ ID NO: 272 - RNA, 22 nt, Homo sapiens
acccuaucaauauugucucugc

SEQ ID NO: 273 - RNA, 23 nt, Homo sapiens
ucucuggagggaagcacuuucug

SEQ ID NO: 274 - RNA, 25 nt, Homo sapiens
cuagugagggacagaaccaggauuc

SEQ ID NO: 275 - RNA, 19 nt, Homo sapiens
gggcgccugugaucccaac

SEQ ID NO: 276 - RNA, 22 nt, Homo sapiens
aagugcuuccuuuuagaggguu

SEQ ID NO: 277 - RNA, 22 nt, Homo sapiens
aggcggggcgccgcgggaccgc

SEQ ID NO: 278 - RNA, 22 nt, Homo sapiens
gugaaauguuuaggaccacuag

SEQ ID NO: 279 - RNA, 25 nt, Homo sapiens
aggggugguguugggacagcuccgu

SEQ ID NO: 280 - RNA, 22 nt, Homo sapiens
uucucaaggaggugucguuuau

SEQ ID NO: 281 - RNA, 22 nt, Homo sapiens
uucaacggguauuuauugagca

SEQ ID NO: 282 - RNA, 22 nt, Homo sapiens
aaaucucugcaggcaaauguga

SEQ ID NO: 283 - RNA, 22 nt, Homo sapiens
cuauacgaccugcugccuuucu

SEQ ID NO: 284 - RNA, 23 nt, Homo sapiens
uguaguguuuccuacuuuaugga

SEQ ID NO: 285 - RNA, 23 nt, Homo sapiens
uaaagugcuuauagugcagguag

SEQ ID NO: 286 - RNA, 22 nt, Homo sapiens
gggagccaggaaguauugaugu

SEQ ID NO: 287 - RNA, 21 nt, Homo sapiens
ucagugcaugacagaacuugg

SEQ ID NO: 288 - RNA, 22 nt, Homo sapiens
ucacaagucaggcucuugggac

SEQ ID NO: 289 - RNA, 21 nt, Homo sapiens
ugguagacuauggaacguagg

SEQ ID NO: 290 - RNA, 23 nt, Homo sapiens
caaagugcucauagugcagguag

SEQ ID NO: 291 - RNA, 23 nt, Homo sapiens
ugugcuugcucgucccgcccgca

SEQ ID NO: 292 - RNA, 23 nt, Homo sapiens
aagugccgccaucuuuugagugu

SEQ ID NO: 293 - RNA, 17 nt, Homo sapiens
uaagugcuuccaugcuu

SEQ ID NO: 294 - RNA, 22 nt, Homo sapiens
aacuguuugcagaggaaacuga

SEQ ID NO: 295 - RNA, 21 nt, Homo sapiens
caacaccagucgaugggcugu

SEQ ID NO: 296 - RNA, 20 nt, Homo sapiens
acugccccaggugcugcugg

SEQ ID NO: 297 - RNA, 21 nt, Homo sapiens
uaccacaggguagaaccacgg

SEQ ID NO: 298 - RNA, 18 nt, Homo sapiens
ugcuuccuuucagagggu

SEQ ID NO: 299 - RNA, 23 nt, Homo sapiens
caacggaaucccaaaagcagcug

SEQ ID NO: 300 - RNA, 21 nt, Homo sapiens
ggcuagcaacagcgcuuaccu

SEQ ID NO: 301 - RNA, 23 nt, Homo sapiens
uuaaugcuaaucgugauaggggu

SEQ ID NO: 302 - RNA, 22 nt, Homo sapiens
ccaauauuacugugcugcuuua

SEQ ID NO: 303 - RNA, 23 nt, Homo sapiens
aguuuugcagguuugcauccagc

SEQ ID NO: 304 - RNA, 23 nt, Homo sapiens
uaagugcuuccauguuugagugu

SEQ ID NO: 305 - RNA, 21 nt, Homo sapiens
agaccuggcccagaccucagc

SEQ ID NO: 306 - RNA, 22 nt, Homo sapiens
ugucuuacucccucaggcacau

SEQ ID NO: 307 - RNA, 22 nt, Homo sapiens
cucaguagccaguguagauccu

SEQ ID NO: 308 - RNA, 21 nt, Homo sapiens
cuguacaggccacugccuugc

SEQ ID NO: 309 - RNA, 23 nt, Homo sapiens
gacacgggcgacagcugcggccc

SEQ ID NO: 310 - RNA, 22 nt, Homo sapiens
cagugcaaugaugaaagggcau

SEQ ID NO: 311 - RNA, 22 nt, Homo sapiens
caaucagcaaguauacugcccu

SEQ ID NO: 312 - RNA, 20 nt, Homo sapiens
uaaggcacgcggugaaugcc

SEQ ID NO: 313 - RNA, 22 nt, Homo sapiens
uacgucaucguugucaucguca

SEQ ID NO: 314 - RNA, 23 nt, Homo sapiens
ucuggcuccgugucuucacuccc

SEQ ID NO: 315 - RNA, 22 nt, Homo sapiens
aaggagcucacagucuauugag

SEQ ID NO: 316 - RNA, 22 nt, Homo sapiens
cuauacaaucuauugccuuccc

SEQ ID NO: 317 - RNA, 22 nt, Homo sapiens
aguuuugcagguuugcauuuca

SEQ ID NO: 318 - RNA, 23 nt, Homo sapiens
uauggcuuuuuauuccuauguga

SEQ ID NO: 319 - RNA, 22 nt, Homo sapiens
ugagguaguagguuguauaguu

SEQ ID NO: 320 - RNA, 21 nt, Homo sapiens
uaaagugcugacagugcagau

SEQ ID NO: 321 - RNA, 22 nt, Homo sapiens
uuggggaaacggccgcugagug

SEQ ID NO: 322 - RNA, 22 nt, Homo sapiens
uucacauugugcuacugucugc

SEQ ID NO: 323 - RNA, 23 nt, Homo sapiens
ccugcagcgacuugauggcuucc

SEQ ID NO: 324 - RNA, 21 nt, Homo sapiens
gcgacccacucuugguuucca

SEQ ID NO: 325 - RNA, 22 nt, Homo sapiens
aaagugcauccuuuuagagguu

SEQ ID NO: 326 - RNA, 22 nt, Homo sapiens
cugugcgugugacagcggcuga

SEQ ID NO: 327 - RNA, 23 nt, Homo sapiens
uagcagcgggaacaguucugcag

SEQ ID NO: 328 - RNA, 21 nt, Homo sapiens
ugacaacuauggaugagcucu

SEQ ID NO: 329 - RNA, 22 nt, Homo sapiens
ccucuagauggaagcacugucu

SEQ ID NO: 330 - RNA, 23 nt, Homo sapiens
aaugacacgaucacucccguuga

SEQ ID NO: 331 - RNA, 22 nt, Homo sapiens
uugcauagucacaaaagugauc

SEQ ID NO: 332 - RNA, 24 nt, Homo sapiens
ucccugagacccuuuaaccuguga

SEQ ID NO: 333 - RNA, 21 nt, Homo sapiens
cuccagagggaaguacuuucu

SEQ ID NO: 334 - RNA, 22 nt, Homo sapiens
gguccagaggggagauagguuc

SEQ ID NO: 335 - RNA, 21 nt, Homo sapiens
ugaguuggccaucugagugag

SEQ ID NO: 336 - RNA, 22 nt, Homo sapiens
uguaaacauccuacacucagcu

SEQ ID NO: 337 - RNA, 22 nt, Homo sapiens
uggaauguaaagaaguauguau

SEQ ID NO: 338 - RNA, 22 nt, Homo sapiens
uauguaacaugguccacuaacu

SEQ ID NO: 339 - RNA, 23 nt, Homo sapiens
guuugcacgggugggccuugucu

SEQ ID NO: 340 - RNA, 22 nt, Homo sapiens
cuccugacuccagguccugugu

SEQ ID NO: 341 - RNA, 22 nt, Homo sapiens
caaccuggaggacuccaugcug

SEQ ID NO: 342 - RNA, 22 nt, Homo sapiens
uacucaggagaguggcaaucac

SEQ ID NO: 343 - RNA, 24 nt, Homo sapiens
agccugauuaaacacaugcucuga

SEQ ID NO: 344 - RNA, 22 nt, Homo sapiens
cuuggcaccuagcaagcacuca

SEQ ID NO: 345 - RNA, 22 nt, Homo sapiens
caucuuaccggacagugcugga

SEQ ID NO: 346 - RNA, 22 nt, Homo sapiens
uuugugaccugguccacuaacc

SEQ ID NO: 347 - RNA, 21 nt, Homo sapiens
cagcagcacacugugguuugu

SEQ ID NO: 348 - RNA, 21 nt, Homo sapiens
cuccagagggaugcacuuucu

SEQ ID NO: 349 - RNA, 22 nt, Homo sapiens
acacagggcuguugugaagacu

SEQ ID NO: 350 - RNA, 22 nt, Homo sapiens
ugccuacugagcugauaucagu

SEQ ID NO: 351 - RNA, 22 nt, Homo sapiens
gaauguugcucggugaaccccu

SEQ ID NO: 352 - RNA, 22 nt, Homo sapiens
ugagguaguagauuguauaguu

SEQ ID NO: 353 - RNA, 20 nt, Homo sapiens
cuguaugcccucaccgcuca

SEQ ID NO: 354 - RNA, 22 nt, Homo sapiens
cauugcacuugucucggucuga

SEQ ID NO: 355 - RNA, 22 nt, Homo sapiens
uuuguucguucggcucgcguga

SEQ ID NO: 356 - RNA, 22 nt, Homo sapiens
uaugugccuuuggacuacaucg

SEQ ID NO: 357 - RNA, 22 nt, Homo sapiens
cuggcccucucugcccuuccgu

SEQ ID NO: 358 - RNA, 22 nt, Homo sapiens
cacgcucaugcacacacccaca

SEQ ID NO: 359 - RNA, 23 nt, Homo sapiens
aggaagcccuggaggggcuggag

SEQ ID NO: 360 - RNA, 22 nt, Homo sapiens
cacccguagaaccgaccuugcg

SEQ ID NO: 361 - RNA, 22 nt, Homo sapiens
gugugcggaaaugcuucugcua

SEQ ID NO: 362 - RNA, 22 nt, Homo sapiens
uugggaucauuuugcauccaua

SEQ ID NO: 363 - RNA, 21 nt, Homo sapiens
uggguuuacguugggagaacu

SEQ ID NO: 364 - RNA, 22 nt, Homo sapiens
gguggcccggccgugccugagg

SEQ ID NO: 365 - RNA, 22 nt, Homo sapiens
ucucugggccugugucuuaggc

SEQ ID NO: 366 - RNA, 22 nt, Homo sapiens
aaucacuaaccacacggccagg

SEQ ID NO: 367 - RNA, 22 nt, Homo sapiens
ugcccuguggacucaguucugg

SEQ ID NO: 368 - RNA, 22 nt, Homo sapiens
uugugucaauaugcgaugaugu

SEQ ID NO: 369 - RNA, 22 nt, Homo sapiens
uguaaacauccccgacuggaag

SEQ ID NO: 370 - RNA, 21 nt, Homo sapiens
aggguaagcugaaccucugau

SEQ ID NO: 371 - RNA, 21 nt, Homo sapiens
aucacauugccagggauuucc

SEQ ID NO: 372 - RNA, 22 nt, Homo sapiens
caguuaucacagugcugaugcu

SEQ ID NO: 373 - RNA, 22 nt, Homo sapiens
uucaccaccuucuccacccagc

SEQ ID NO: 374 - RNA, 22 nt, Homo sapiens
aaucauacagggacauccaguu

SEQ ID NO: 375 - RNA, 22 nt, Homo sapiens
aagugcugucauagcugagguc

SEQ ID NO: 376 - RNA, 22 nt, Homo sapiens
acaaagugcuucccuuuagagu

SEQ ID NO: 377 - RNA, 22 nt, Homo sapiens
uauugcacucgucccggccucc

SEQ ID NO: 378 - RNA, 23 nt, Homo sapiens
agcugguguugugaaucaggccg

SEQ ID NO: 379 - RNA, 22 nt, Homo sapiens
uagguaguuucauguuguuggg

SEQ ID NO: 380 - RNA, 21 nt, Homo sapiens
aauggcgccacuaggguugug

SEQ ID NO: 381 - RNA, 22 nt, Homo sapiens
cuguacagccuccuagcuuucc

SEQ ID NO: 382 - RNA, 23 nt, Homo sapiens
ucaaaugcucagacuccuguggu

SEQ ID NO: 383 - RNA, 23 nt, Homo sapiens
cagugcaaugauauugucaaagc

SEQ ID NO: 384 - RNA, 21 nt, Homo sapiens
gaacggcuucauacaggaguu

SEQ ID NO: 385 - RNA, 22 nt, Homo sapiens
aguauucuguaccagggaaggu

SEQ ID NO: 386 - RNA, 22 nt, Homo sapiens
gaggguuggguggaggcucucc

SEQ ID NO: 387 - RNA, 22 nt, Homo sapiens
ugagguaguaguuugugcuguu

SEQ ID NO: 388 - RNA, 22 nt, Homo sapiens
gugacaucacauauacggcagc

SEQ ID NO: 389 - RNA, 22 nt, Homo sapiens
agacccuggucugcacucuauc

SEQ ID NO: 390 - RNA, 22 nt, Homo sapiens
cgaaucauuauuugcugcucua

SEQ ID NO: 391 - RNA, 20 nt, Homo sapiens
guguguggaaaugcuucugc

SEQ ID NO: 392 - RNA, 22 nt, Homo sapiens
guagauucuccuucuaugagua

SEQ ID NO: 393 - RNA, 22 nt, Homo sapiens
acggguuaggcucuugggagcu

SEQ ID NO: 394 - RNA, 22 nt, Homo sapiens
ccucugaaauucaguucuucag

SEQ ID NO: 395 - RNA, 22 nt, Homo sapiens
ggcuacaacacaggacccgggc

SEQ ID NO: 396 - RNA, 23 nt, Homo sapiens
uaagugcuuccauguuucagugg

SEQ ID NO: 397 - RNA, 21 nt, Homo sapiens
aaagugcuuccuuuuagaggg

SEQ ID NO: 398 - RNA, 22 nt, Homo sapiens
caaagcgcuccccuuuagaggu

SEQ ID NO: 399 - RNA, 23 nt, Homo sapiens
cgggucggaguuagcucaagcgg

SEQ ID NO: 400 - RNA, 23 nt, Homo sapiens
aggcaguguaguuagcugauugc

SEQ ID NO: 401 - RNA, 22 nt, Homo sapiens
uagcagcacguaaauauuggcg

SEQ ID NO: 402 - RNA, 22 nt, Homo sapiens
cuuucagucggauguuuacagc

SEQ ID NO: 403 - RNA, 24 nt, Homo sapiens
aaagacauaggauagagucaccuc

SEQ ID NO: 404 - RNA, 21 nt, Homo sapiens
cucccacaugcaggguuugca

SEQ ID NO: 405 - RNA, 21 nt, Homo sapiens
cccggagccaggaugcagcuc

SEQ ID NO: 406 - RNA, 22 nt, Homo sapiens
agggacgggacgcggugcagug

SEQ ID NO: 407 - RNA, 22 nt, Homo sapiens
aaaaguaauugcgaguuuuacc

SEQ ID NO: 408 - RNA, 23 nt, Homo sapiens
uuuggcacuagcacauuuuugcu

SEQ ID NO: 409 - RNA, 21 nt, Homo sapiens
aucacauugccagggauuacc

SEQ ID NO: 410 - RNA, 22 nt, Homo sapiens
agaguugagucuggacgucccg

SEQ ID NO: 411 - RNA, 23 nt, Homo sapiens
ccucagggcuguagaacagggcu

SEQ ID NO: 412 - RNA, 22 nt, Homo sapiens
aaaaguaauugcggucuuuggu

SEQ ID NO: 413 - RNA, 22 nt, Homo sapiens
aaacaaacauggugcacuucuu

SEQ ID NO: 414 - RNA, 22 nt, Homo sapiens
cuagguauggucccagggaucc

SEQ ID NO: 415 - RNA, 23 nt, Homo sapiens
uaggcagugucauuagcugauug

SEQ ID NO: 416 - RNA, 23 nt, Homo sapiens
uaauccuugcuaccugggugaga

SEQ ID NO: 417 - RNA, 22 nt, Homo sapiens
uggucuaggauuguuggaggag

SEQ ID NO: 418 - RNA, 22 nt, Homo sapiens
auguagggcuaaaagccauggg

SEQ ID NO: 419 - RNA, 22 nt, Homo sapiens
ugagguaggagguuguauaguu

SEQ ID NO: 420 - RNA, 22 nt, Homo sapiens
uggugguuuacaaaguaauuca

SEQ ID NO: 421 - RNA, 22 nt, Homo sapiens
acugauuucuuuugguguucag

SEQ ID NO: 422 - RNA, 24 nt, Homo sapiens
uucuccaaaagaaagcacuuucug

SEQ ID NO: 423 - RNA, 22 nt, Homo sapiens
aaucaugugcagugccaauaug

SEQ ID NO: 424 - RNA, 22 nt, Homo sapiens
uauguaacacgguccacuaacc

SEQ ID NO: 425 - RNA, 22 nt, Homo sapiens
caggccauauugugcugccuca

SEQ ID NO: 426 - RNA, 21 nt, Homo sapiens
agggcccccccucaauccugu

SEQ ID NO: 427 - RNA, 22 nt, Homo sapiens
aacgccauuaucacacuaaaua

SEQ ID NO: 428 - RNA, 22 nt, Homo sapiens
aacaucacagcaagucugugcu

SEQ ID NO: 429 - RNA, 22 nt, Homo sapiens
uggugggccgcagaacaugugc

SEQ ID NO: 430 - RNA, 22 nt, Homo sapiens
ucuucucuguuuuggccaugug

SEQ ID NO: 431 - RNA, 22 nt, Homo sapiens
ugaguauuacauggccaaucuc

SEQ ID NO: 432 - RNA, 21 nt, Homo sapiens
aacauagaggaaauuccacgu

SEQ ID NO: 433 - RNA, 22 nt, Homo sapiens
cugcaauguaagcacuucuuac

SEQ ID NO: 434 - RNA, 22 nt, Homo sapiens
ugagguaguagguuguaugguu

SEQ ID NO: 435 - RNA, 22 nt, Homo sapiens
ggggguccccggugcucggauc

SEQ ID NO: 436 - RNA, 22 nt, Homo sapiens
acaggugagguucuugggagcc

SEQ ID NO: 437 - RNA, 22 nt, Homo sapiens
aaacauucgcggugcacuucuu

SEQ ID NO: 438 - RNA, 22 nt, Homo sapiens
ucaggcucaguccccucccgau

SEQ ID NO: 439 - RNA, 21 nt, Homo sapiens
auccuugcuaucugggugcua

SEQ ID NO: 440 - RNA, 23 nt, Homo sapiens
ugugcaaauccaugcaaaacuga

SEQ ID NO: 441 - RNA, 23 nt, Homo sapiens
gaacgcgcuucccuauagagggu

SEQ ID NO: 442 - RNA, 22 nt, Homo sapiens
uccgagccugggucucccucuu

SEQ ID NO: 443 - RNA, 19 nt, Homo sapiens
aggcacggugucagcaggc

SEQ ID NO: 444 - RNA, 22 nt, Homo sapiens
cuggacugagccgugcuacugg

SEQ ID NO: 445 - RNA, 21 nt, Homo sapiens
acucuuucccuguugcacuac

SEQ ID NO: 446 - RNA, 22 nt, Homo sapiens
cuuucagucggauguuugcagc

SEQ ID NO: 447 - RNA, 22 nt, Homo sapiens
ugauugguacgucuguggguag

SEQ ID NO: 448 - RNA, 23 nt, Homo sapiens
acuucaccugguccacuagccgu

SEQ ID NO: 449 - RNA, 22 nt, Homo sapiens
cucuagagggaagcacuuucug

SEQ ID NO: 450 - RNA, 22 nt, Homo sapiens
cagugccucggcagugcagccc

SEQ ID NO: 451 - RNA, 20 nt, Homo sapiens
guagaggagauggcgcaggg

SEQ ID NO: 452 - RNA, 23 nt, Homo sapiens
ccuaguagguguccaguaagugu

SEQ ID NO: 453 - RNA, 22 nt, Homo sapiens
ucccugagacccuaacuuguga

SEQ ID NO: 454 - RNA, 23 nt, Homo sapiens
gagggucuugggagggaugugac

SEQ ID NO: 455 - RNA, 23 nt, Homo sapiens
agcagcauuguacagggcuauca

SEQ ID NO: 456 - RNA, 21 nt, Homo sapiens
uugaaaggcuauuucuugguc

SEQ ID NO: 457 - RNA, 22 nt, Homo sapiens
acugcugagcuagcacuucccg

SEQ ID NO: 458 - RNA, 23 nt, Homo sapiens
uucucgaggaaagaagcacuuuc

SEQ ID NO: 459 - RNA, 22 nt, Homo sapiens
gugaacgggcgccaucccgagg

SEQ ID NO: 460 - RNA, 22 nt, Homo sapiens
uccauuacacuacccugccucu

SEQ ID NO: 461 - RNA, 22 nt, Homo sapiens
gacugacaccucuuugggugaa

SEQ ID NO: 462 - RNA, 22 nt, Homo sapiens
uggagagaaaggcaguuccuga

SEQ ID NO: 463 - RNA, 22 nt, Homo sapiens
gcuauuucacgacaccaggguu

SEQ ID NO: 464 - RNA, 23 nt, Homo sapiens
gcagcagagaauaggacuacguc

SEQ ID NO: 465 - RNA, 22 nt, Homo sapiens
cgucuuacccagcaguguuugg

SEQ ID NO: 466 - RNA, 23 nt, Homo sapiens
ugauuguagccuuuuggaguaga

SEQ ID NO: 467 - RNA, 22 nt, Homo sapiens
uggcaguguauuguuagcuggu

SEQ ID NO: 468 - RNA, 23 nt, Homo sapiens
uaauacugccggguaaugaugga

SEQ ID NO: 469 - RNA, 23 nt, Homo sapiens
guccaguuuucccaggaaucccu

SEQ ID NO: 470 - RNA, 21 nt, Homo sapiens
uugugcuugaucuaaccaugu

SEQ ID NO: 471 - RNA, 22 nt, Homo sapiens
caagaaccucaguugcuuuugu

SEQ ID NO: 472 - RNA, 22 nt, Homo sapiens
uggcagugucuuagcugguugu

SEQ ID NO: 473 - RNA, 22 nt, Homo sapiens
uccuucauuccaccggagucug

SEQ ID NO: 474 - RNA, 23 nt, Homo sapiens
agcucggucugaggccccucagu

SEQ ID NO: 475 - RNA, 22 nt, Homo sapiens
aaucguacagggucauccacuu

SEQ ID NO: 476 - RNA, 23 nt, Homo sapiens
aaggagcuuacaaucuagcuggg

SEQ ID NO: 477 - RNA, 22 nt, Homo sapiens
aagugccuccuuuuagaguguu

SEQ ID NO: 478 - RNA, 21 nt, Homo sapiens
ugagcuaaaugugugcuggga

SEQ ID NO: 479 - RNA, 20 nt, Homo sapiens
acucaaacugugggggcacu

SEQ ID NO: 480 - RNA, 23 nt, Homo sapiens
cccaguguucagacuaccuguuc

SEQ ID NO: 481 - RNA, 21 nt, Homo sapiens
cccagauaauggcacucucaa

SEQ ID NO: 482 - RNA, 18 nt, Homo sapiens
aucccaccucugccacca

SEQ ID NO: 483 - RNA, 22 nt, Homo sapiens
aaagugcuuccuuuuagagggu

SEQ ID NO: 484 - RNA, 22 nt, Homo sapiens
acucaaaacccuucagugacuu

SEQ ID NO: 485 - RNA, 22 nt, Homo sapiens
acuccagccccacagccucagc

SEQ ID NO: 486 - RNA, 22 nt, Homo sapiens
caucuuccaguacaguguugga

SEQ ID NO: 487 - RNA, 21 nt, Homo sapiens
acagucugcugagguuggagc

SEQ ID NO: 488 - RNA, 22 nt, Homo sapiens
acugcagugaaggcacuuguag

SEQ ID NO: 489 - RNA, 22 nt, Homo sapiens
uacugcagacguggcaaucaug

SEQ ID NO: 490 - RNA, 22 nt, Homo sapiens
uaacacugucugguaaagaugg

SEQ ID NO: 491 - RNA, 22 nt, Homo sapiens
uugagaaugaugaaucauuagg

SEQ ID NO: 492 - RNA, 22 nt, Homo sapiens
aucgugcaucccuuuagagugu

SEQ ID NO: 493 - RNA, 22 nt, Homo sapiens
uucccuuugucauccuaugccu

SEQ ID NO: 494 - RNA, 21 nt, Homo sapiens
aucauagaggaaaauccacgu

SEQ ID NO: 495 - RNA, 22 nt, Homo sapiens
uuuuucauuauugcuccugacc

SEQ ID NO: 496 - RNA, 22 nt, Homo sapiens
acagcaggcacagacaggcagu

SEQ ID NO: 497 - RNA, 23 nt, Homo sapiens
ucucacacagaaaucgcacccgu

SEQ ID NO: 498 - RNA, 20 nt, Homo sapiens
ccucugggcccuuccuccag

SEQ ID NO: 499 - RNA, 23 nt, Homo sapiens
ucuuugguuaucuagcuguauga

SEQ ID NO: 500 - RNA, 22 nt, Homo sapiens
acagauucgauucuaggggaau

SEQ ID NO: 501 - RNA, 22 nt, Homo sapiens
uggguuccuggcaugcugauuu

SEQ ID NO: 502 - RNA, 21 nt, Homo sapiens
aggggugcuaucugugauuga

SEQ ID NO: 503 - RNA, 22 nt, Homo sapiens
aggcaguguauuguuagcuggc

SEQ ID NO: 504 - RNA, 22 nt, Homo sapiens
uagguuauccguguugccuucg

SEQ ID NO: 505 - RNA, 22 nt, Homo sapiens
uuuugcgauguguuccuaauau

SEQ ID NO: 506 - RNA, 22 nt, Homo sapiens
caagcucgcuucuaugggucug

SEQ ID NO: 507 - RNA, 22 nt, Homo sapiens
aacccguagauccgaucuugug

SEQ ID NO: 508 - RNA, 25 nt, Homo sapiens
ggcggagggaaguagguccguuggu

SEQ ID NO: 509 - RNA, 23 nt, Homo sapiens
acugcccuaagugcuccuucugg

SEQ ID NO: 510 - RNA, 22 nt, Homo sapiens
aaaagcuggguugagagggcaa

SEQ ID NO: 511 - RNA, 21 nt, Homo sapiens
agagaagaagaucagccugca

SEQ ID NO: 512 - RNA, 22 nt, Homo sapiens
uuagggcccuggcuccaucucc

SEQ ID NO: 513 - RNA, 22 nt, Homo sapiens
uggauuucuuugugaaucacca

SEQ ID NO: 514 - RNA, 22 nt, Homo sapiens
cuguugccacuaaccucaaccu

SEQ ID NO: 515 - RNA, 22 nt, Homo sapiens
cguguauuugacaagcugaguu

SEQ ID NO: 516 - RNA, 23 nt, Homo sapiens
aacauucauugcugucggugggu

SEQ ID NO: 517 - RNA, 21 nt, Homo sapiens
uaguagaccguauagcguacg

SEQ ID NO: 518 - RNA, 23 nt, Homo sapiens
agcuacauugucugcuggguuuc

SEQ ID NO: 519 - RNA, 21 nt, Homo sapiens
augauccaggaaccugccucu

SEQ ID NO: 520 - RNA, 21 nt, Homo sapiens
cuuuuugcggucugggcuugc

SEQ ID NO: 521 - RNA, 22 nt, Homo sapiens
caagcuuguaucuauagguaug

SEQ ID NO: 522 - RNA, 22 nt, Homo sapiens
acaguagucugcacauugguua

SEQ ID NO: 523 - RNA, 20 nt, Homo sapiens
ucacuguucagacaggcgga

SEQ ID NO: 524 - RNA, 23 nt, Homo sapiens
ugucugcccgcaugccugccucu

SEQ ID NO: 525 - RNA, 21 nt, Homo sapiens
uaaggcacccuucugaguaga

SEQ ID NO: 526 - RNA, 22 nt, Homo sapiens
cagugguuuuacccuaugguag

SEQ ID NO: 527 - RNA, 21 nt, Homo sapiens
caaaacgugaggcgcugcuau

SEQ ID NO: 528 - RNA, 19 nt, Homo sapiens
gugucugcuuccuguggga

SEQ ID NO: 529 - RNA, 21 nt, Homo sapiens
ccuguugaaguguaaucccca

SEQ ID NO: 530 - RNA, 22 nt, Homo sapiens
ugguuuaccgucccacauacau

SEQ ID NO: 531 - RNA, 21 nt, Homo sapiens
cugacuguugccguccuccag

SEQ ID NO: 532 - RNA, 19 nt, Homo sapiens
aagcagcugccucugaggc

SEQ ID NO: 533 - RNA, 22 nt, Homo sapiens
ucgugcaucccuuuagaguguu

SEQ ID NO: 534 - RNA, 20 nt, Homo sapiens
cggcucugggucugugggga

SEQ ID NO: 535 - RNA, 25 nt, Homo sapiens
aggcaccagccaggcauugcucagc

SEQ ID NO: 536 - RNA, 21 nt, Homo sapiens
agcuacaucuggcuacugggu

SEQ ID NO: 537 - RNA, 22 nt, Homo sapiens
accguggcuuucgauuguuacu

SEQ ID NO: 538 - RNA, 22 nt, Homo sapiens
ugagaacugaauuccauaggcu

SEQ ID NO: 539 - RNA, 23 nt, Homo sapiens
caaagcgcuucucuuuagagugu

SEQ ID NO: 540 - RNA, 22 nt, Homo sapiens
uagguaguuuccuguuguuggg

SEQ ID NO: 541 - RNA, 21 nt, Homo sapiens
gcuaguccugacucagccagu

SEQ ID NO: 542 - RNA, 22 nt, Homo sapiens
ugaaggucuacugugugccagg

SEQ ID NO: 543 - RNA, 22 nt, Homo sapiens
aucuggagguaagaagcacuuu

SEQ ID NO: 544 - RNA, 22 nt, Homo sapiens
gggguuccuggggaugggauuu

SEQ ID NO: 545 - RNA, 23 nt, Homo sapiens
agguugggaucgguugcaaugcu

SEQ ID NO: 546 - RNA, 22 nt, Homo sapiens
cuuagcagguuguauuaucauu

SEQ ID NO: 547 - RNA, 22 nt, Homo sapiens
gcuacuucacaacaccagggcc

SEQ ID NO: 548 - RNA, 23 nt, Homo sapiens
aaaagugcuuacagugcagguag

SEQ ID NO: 549 - RNA, 22 nt, Homo sapiens
agacuucccauuugaagguggc

SEQ ID NO: 550 - RNA, 22 nt, Homo sapiens
ugagguaguaguuuguacaguu

SEQ ID NO: 551 - RNA, 23 nt, Homo sapiens
aacauucaacgcugucggugagu

SEQ ID NO: 552 - RNA, 22 nt, Homo sapiens
caggucgucuugcagggcuucu

SEQ ID NO: 553 - RNA, 22 nt, Homo sapiens
uuaugguuugccugggacugag

SEQ ID NO: 554 - RNA, 22 nt, Homo sapiens
acuguaguaugggcacuuccag

SEQ ID NO: 555 - RNA, 22 nt, Homo sapiens
ggugcagugcugcaucucuggu

SEQ ID NO: 556 - RNA, 21 nt, Homo sapiens
cgcgggugcuuacugacccuu

SEQ ID NO: 557 - RNA, 22 nt, Homo sapiens
uagaguuacacccugggaguua

SEQ ID NO: 558 - RNA, 23 nt, Homo sapiens
cacccggcugugugcacaugugc

SEQ ID NO: 559 - RNA, 22 nt, Homo sapiens
ugccugucuacacuugcugugc

SEQ ID NO: 560 - RNA, 21 nt, Homo sapiens
cuagacugaagcuccuugagg

SEQ ID NO: 561 - RNA, 21 nt, Homo sapiens
cuccguuugccuguuucgcug

SEQ ID NO: 562 - RNA, 23 nt, Homo sapiens
aucgcugcgguugcgagcgcugu

SEQ ID NO: 563 - RNA, 22 nt, Homo sapiens
ugaaacauacacgggaaaccuc

SEQ ID NO: 564 - RNA, 22 nt, Homo sapiens
gugaauuaccgaagggccauaa

SEQ ID NO: 565 - RNA, 22 nt, Homo sapiens
caacaaaucccagucuaccuaa

SEQ ID NO: 566 - RNA, 23 nt, Homo sapiens
uagugcaauauugcuuauagggu

SEQ ID NO: 567 - RNA, 22 nt, Homo sapiens
ccaaaacugcaguuacuuuugc

SEQ ID NO: 568 - RNA, 21 nt, Homo sapiens
cauuauuacuuuugguacgcg

SEQ ID NO: 569 - RNA, 22 nt, Homo sapiens
ugcccuuaaaggugaacccagu

SEQ ID NO: 570 - RNA, 22 nt, Homo sapiens
uauguaauaugguccacaucuu

SEQ ID NO: 571 - RNA, 21 nt, Homo sapiens
cggcggggacggcgauugguc

SEQ ID NO: 572 - RNA, 22 nt, Homo sapiens
gcugacuccuaguccagggcuc

SEQ ID NO: 573 - RNA, 22 nt, Homo sapiens
aaaaguaaucgcgguuuuuguc

SEQ ID NO: 574 - RNA, 22 nt, Homo sapiens
aacuggccuacaaagucccagu

SEQ ID NO: 575 - RNA, 23 nt, Homo sapiens
uggaagacuagugauuuuguugu

SEQ ID NO: 576 - RNA, 23 nt, Homo sapiens
ugaggggcagagagcgagacuuu

SEQ ID NO: 577 - RNA, 21 nt, Homo sapiens
auauaugaugacuuagcuuuu

SEQ ID NO: 578 - RNA, 23 nt, Homo sapiens
ugaguaccgccaugucuguuggg

SEQ ID NO: 579 - RNA, 23 nt, Homo sapiens
uaaaucccauggugccuucuccu

SEQ ID NO: 580 - RNA, 23 nt, Homo sapiens
uaaauuucaccuuucugagaagg

SEQ ID NO: 581 - RNA, 21 nt, Homo sapiens
augaccuaugaauugacagac

SEQ ID NO: 582 - RNA, 20 nt, Homo sapiens
caccaggcauuguggucucc

SEQ ID NO: 583 - RNA, 23 nt, Homo sapiens
uacccuguagauccgaauuugug

SEQ ID NO: 584 - RNA, 22 nt, Homo sapiens
uggacggagaacugauaagggu

SEQ ID NO: 585 - RNA, 22 nt, Homo sapiens
auucuaauuucuccacgucuuu

SEQ ID NO: 586 - RNA, 23 nt, Homo sapiens
aucaacagacauuaauugggcgc

SEQ ID NO: 587 - RNA, 23 nt, Homo sapiens
gaagugcuucgauuuuggggugu

SEQ ID NO: 588 - RNA, 23 nt, Homo sapiens
guguuaauuaaaccucuauuuac

SEQ ID NO: 589 - RNA, 22 nt, Homo sapiens
aagcugccaguugaagaacugu

SEQ ID NO: 590 - RNA, 23 nt, Homo sapiens
uguaaacauccuacacucucagc

SEQ ID NO: 591 - RNA, 22 nt, Homo sapiens
auauaauacaaccugcuaagug

SEQ ID NO: 592 - RNA, 23 nt, Homo sapiens
agcuucuuuacagugcugccuug

SEQ ID NO: 593 - RNA, 23 nt, Homo sapiens
uacccuguagaaccgaauuugug

SEQ ID NO: 594 - RNA, 22 nt, Homo sapiens
aaagugcauccuuuuagagugu

SEQ ID NO: 595 - RNA, 21 nt, Homo sapiens
aaaacggugagauuuuguuuu

SEQ ID NO: 596 - RNA, 20 nt, Homo sapiens
aggguguuucucucaucucu

SEQ ID NO: 597 - RNA, 22 nt, Homo sapiens
augcugacauauuuacuagagg

SEQ ID NO: 598 - RNA, 23 nt, Homo sapiens
cggcccgggcugcugcuguuccu

SEQ ID NO: 599 - RNA, 22 nt, Homo sapiens
uggaauguaaggaagugugugg

SEQ ID NO: 600 - RNA, 23 nt, Homo sapiens
ugugcaaaucuaugcaaaacuga

SEQ ID NO: 601 - RNA, 24 nt, Homo sapiens
aauccuuggaaccuaggugugagu

SEQ ID NO: 602 - RNA, 22 nt, Homo sapiens
ucgacagcacgacacugccuuc

SEQ ID NO: 603 - RNA, 22 nt, Homo sapiens
auaaagcuagauaaccgaaagu

SEQ ID NO: 604 - RNA, 21 nt, Homo sapiens
ccaccaccgugucugacacuu

SEQ ID NO: 605 - RNA, 22 nt, Homo sapiens
uaaugccccuaaaaauccuuau

SEQ ID NO: 606 - RNA, 22 nt, Homo sapiens
gcccgcguguggagccaggugu

SEQ ID NO: 607 - RNA, 21 nt, Homo sapiens
aagcauucuuucauugguugg

SEQ ID NO: 608 - RNA, 22 nt, Homo sapiens
uaguaccaguaccuuguguuca

SEQ ID NO: 609 - RNA, 21 nt, Homo sapiens
ucacagugaaccggucucuuu

SEQ ID NO: 610 - RNA, 23 nt, Homo sapiens
uucauuugguauaaaccgcgauu

SEQ ID NO: 611 - RNA, 21 nt, Homo sapiens
caaagcgcuucccuuuggagc

SEQ ID NO: 612 - RNA, 23 nt, Homo sapiens
aaaauggugcccuagugacuaca

SEQ ID NO: 613 - RNA, 22 nt, Homo sapiens
gaaaucaagcgugggugagacc

SEQ ID NO: 614 - RNA, 22 nt, Homo sapiens
cagccacaacuacccugccacu

SEQ ID NO: 615 - RNA, 21 nt, Homo sapiens
gugcauuguaguugcauugca

SEQ ID NO: 616 - RNA, 22 nt, Homo sapiens
caaauucguaucuaggggaaua

SEQ ID NO: 617 - RNA, 21 nt, Homo sapiens
uacuuggaaaggcaucaguug

SEQ ID NO: 618 - RNA, 23 nt, Homo sapiens
caguaacaaagauucauccuugu

SEQ ID NO: 619 - RNA, 22 nt, Homo sapiens
auaagacgaacaaaagguuugu

SEQ ID NO: 620 - RNA, 20 nt, Homo sapiens
auggagauagauauagaaau

SEQ ID NO: 621 - RNA, 23 nt, Homo sapiens
agugccugagggaguaagagccc

SEQ ID NO: 622 - RNA, 21 nt, Homo sapiens
ucuaguaagaguggcagucga

SEQ ID NO: 623 - RNA, 22 nt, Homo sapiens
ugagguaguaaguuguauuguu

SEQ ID NO: 624 - RNA, 21 nt, Homo sapiens
caagucacuagugguuccguu

SEQ ID NO: 625 - RNA, 22 nt, Homo sapiens
cugggagaaggcuguuuacucu

SEQ ID NO: 626 - RNA, 22 nt, Homo sapiens
uugcauauguaggaugucccau

SEQ ID NO: 627 - RNA, 22 nt, Homo sapiens
ggaggggucccgcacugggagg

SEQ ID NO: 628 - RNA, 21 nt, Homo sapiens
auugacacuucugugaguaga

SEQ ID NO: 629 - RNA, 22 nt, Homo sapiens
auucugcauuuuuagcaaguuc

SEQ ID NO: 630 - RNA, 22 nt, Homo sapiens
gacuauagaacuuucccccuca

SEQ ID NO: 631 - RNA, 22 nt, Homo sapiens
aauccuuugucccugggugaga

SEQ ID NO: 632 - RNA, 21 nt, Homo sapiens
guucaaauccagaucuauaac

SEQ ID NO: 633 - RNA, 22 nt, Homo sapiens
uaauacugccugguaaugauga

SEQ ID NO: 634 - RNA, 22 nt, Homo sapiens
gagugccuucuuuuggagcguu

SEQ ID NO: 635 - RNA, 22 nt, Homo sapiens
uauggcacugguagaauucacu

SEQ ID NO: 636 - RNA, 21 nt, Homo sapiens
auguaugugugcaugugcaug

SEQ ID NO: 637 - RNA, 22 nt, Homo sapiens
agggacuuucaggggcagcugu

SEQ ID NO: 638 - RNA, 23 nt, Homo sapiens
uuauugcuuaagaauacgcguag

SEQ ID NO: 639 - RNA, 21 nt, Homo sapiens
uuggccacaauggguuagaac

SEQ ID NO: 640 - RNA, 24 nt, Homo sapiens
ugccugggucucuggccugcgcgu

SEQ ID NO: 641 - RNA, 22 nt, Homo sapiens
cagugcaauguuaaaagggcau

SEQ ID NO: 642 - RNA, 22 nt, Homo sapiens
uuauaaagcaaugagacugauu

SEQ ID NO: 643 - RNA, 22 nt, Homo sapiens
ucucccaacccuuguaccagug

SEQ ID NO: 644 - RNA, 23 nt, Homo sapiens
ugguuguaguccgugcgagaaua

SEQ ID NO: 645 - RNA, 22 nt, Homo sapiens
ugcggggcuagggcuaacagca

SEQ ID NO: 646 - RNA, 22 nt, Homo sapiens
cucccacugcuucacuugacua

SEQ ID NO: 647 - RNA, 22 nt, Homo sapiens
ugggucuuugcgggcgagauga

SEQ ID NO: 648 - RNA, 21 nt, Homo sapiens
uagauaaaauauugguaccug

SEQ ID NO: 649 - RNA, 21 nt, Homo sapiens
ugauauguuugauauuggguu

SEQ ID NO: 650 - RNA, 22 nt, Homo sapiens
cugggagguggauguuuacuuc

SEQ ID NO: 651 - RNA, 21 nt, Homo sapiens
guguugaaacaaucucuacug

SEQ ID NO: 652 - RNA, 22 nt, Homo sapiens
ggauaucaucauauacuguaag

SEQ ID NO: 653 - RNA, 22 nt, Homo sapiens
cucuagagggaagcacuuucuc

SEQ ID NO: 654 - RNA, 22 nt, Homo sapiens
cccugugcccggcccacuucug

SEQ ID NO: 655 - RNA, 22 nt, Homo sapiens
ucugcccccuccgcugcugcca

SEQ ID NO: 656 - RNA, 22 nt, Homo sapiens
agaauuguggcuggacaucugu

SEQ ID NO: 657 - RNA, 22 nt, Homo sapiens
ggagaaauuauccuuggugugu

SEQ ID NO: 658 - RNA, 22 nt, Homo sapiens
ccuauucuugauuacuuguuuc

SEQ ID NO: 659 - RNA, 21 nt, Homo sapiens
uacucaaaaagcugucaguca

SEQ ID NO: 660 - RNA, 22 nt, Homo sapiens
ucagcaaacauuuauugugugc

SEQ ID NO: 661 - RNA, 23 nt, Homo sapiens
uagcaccauuugaaaucaguguu

SEQ ID NO: 662 - RNA, 22 nt, Homo sapiens
auaagacgagcaaaaagcuugu

SEQ ID NO: 663 - RNA, 22 nt, Homo sapiens
caacuagacugugagcuucuag

SEQ ID NO: 664 - RNA, 21 nt, Homo sapiens
uccugcgcgucccagaugccc

SEQ ID NO: 665 - RNA, 22 nt, Homo sapiens
aacauucaaccugucggugagu

SEQ ID NO: 666 - RNA, 20 nt, Homo sapiens
cuacaaagggaagcccuuuc

SEQ ID NO: 667 - RNA, 24 nt, Homo sapiens
agccuggaagcuggagccugcagu

SEQ ID NO: 668 - RNA, 21 nt, Homo sapiens
auuugugcuuggcucugucac

SEQ ID NO: 669 - RNA, 23 nt, Homo sapiens
cagugcaauaguauugucaaagc

SEQ ID NO: 670 - RNA, 22 nt, Homo sapiens
ugagaacugaauuccauggguu

SEQ ID NO: 671 - RNA, 22 nt, Homo sapiens
aaaaguaauugugguuuuugcc

SEQ ID NO: 672 - RNA, 22 nt, Homo sapiens
uauacaagggcaagcucucugu

SEQ ID NO: 673 - RNA, 22 nt, Homo sapiens
augguuccgucaagcaccaugg

SEQ ID NO: 674 - RNA, 22 nt, Homo sapiens
uacccagagcaugcagugugaa

SEQ ID NO: 675 - RNA, 21 nt, Homo sapiens
uggcagggaggcugggagggg

SEQ ID NO: 676 - RNA, 22 nt, Homo sapiens
cgaaaacagcaauuaccuuugc

SEQ ID NO: 677 - RNA, 22 nt, Homo sapiens
aguggggaacccuuccaugagg

SEQ ID NO: 678 - RNA, 20 nt, Homo sapiens
guccgcucggcgguggccca

SEQ ID NO: 679 - RNA, 22 nt, Homo sapiens
caaaaaucucaauuacuuuugc

SEQ ID NO: 680 - RNA, 22 nt, Homo sapiens
uagcaccaucugaaaucgguua

SEQ ID NO: 681 - RNA, 23 nt, Homo sapiens
acuuaaacguggauguacuugcu

SEQ ID NO: 682 - RNA, 22 nt, Homo sapiens
cgcaggggccgggugcucaccg

SEQ ID NO: 683 - RNA, 22 nt, Homo sapiens
agaaggaaauugaauucauuua

SEQ ID NO: 684 - RNA, 22 nt, Homo sapiens
uaugugggaugguaaaccgcuu

SEQ ID NO: 685 - RNA, 22 nt, Homo sapiens
acucaaaaugggggcgcuuucc

SEQ ID NO: 686 - RNA, 22 nt, Homo sapiens
aacacaccuauucaaggauuca

SEQ ID NO: 687 - RNA, 22 nt, Homo sapiens
aacgcacuucccuuuagagugu

SEQ ID NO: 688 - RNA, 22 nt, Homo sapiens
uaacacugucugguaacgaugu

SEQ ID NO: 689 - RNA, 22 nt, Homo sapiens
ucaggccaggcacaguggcuca

SEQ ID NO: 690 - RNA, 20 nt, Homo sapiens
accaggaggcugaggccccu

SEQ ID NO: 691 - RNA, 21 nt, Homo sapiens
caaagguauuugugguuuuug

SEQ ID NO: 692 - RNA, 19 nt, Homo sapiens
agcugucugaaaaugucuu

SEQ ID NO: 693 - RNA, 20 nt, Homo sapiens
auuccuagaaauuguucaua

SEQ ID NO: 694 - RNA, 22 nt, Homo sapiens
uguaaacauccuugacuggaag

SEQ ID NO: 695 - RNA, 23 nt, Homo sapiens
caaagugcuguucgugcagguag

SEQ ID NO: 696 - RNA, 22 nt, Homo sapiens
agaucagaaggugauuguggcu

SEQ ID NO: 697 - RNA, 22 nt, Homo sapiens
aaaaccgucuaguuacaguugu

SEQ ID NO: 698 - RNA, 22 nt, Homo sapiens
aaaaguauuugcggguuuuguc

SEQ ID NO: 699 - RNA, 22 nt, Homo sapiens
uccagcaucagugauuuuguug

SEQ ID NO: 700 - RNA, 22 nt, Homo sapiens
gucccucuccaaaugugucuug

SEQ ID NO: 701 - RNA, 22 nt, Homo sapiens
cugggagaggguuguuuacucc

SEQ ID NO: 702 - RNA, 21 nt, Homo sapiens
cauaaaguagaaagcacuacu

SEQ ID NO: 703 - RNA, 22 nt, Homo sapiens
caacaaaucacagucugccaua

SEQ ID NO: 704 - RNA, 22 nt, Homo sapiens
uucaaguaauccaggauaggcu

SEQ ID NO: 705 - RNA, 23 nt, Homo sapiens
uauucauuuauccccagccuaca

SEQ ID NO: 706 - RNA, 22 nt, Homo sapiens
aauugcacgguauccaucugua

SEQ ID NO: 707 - RNA, 22 nt, Homo sapiens
uacccauugcauaucggaguug

SEQ ID NO: 708 - RNA, 22 nt, Homo sapiens
caaaguuuaagauccuugaagu

SEQ ID NO: 709 - RNA, 22 nt, Homo sapiens
uagcaccauuugaaaucgguua

SEQ ID NO: 710 - RNA, 22 nt, Homo sapiens
uuccuaugcauauacuucuuug

SEQ ID NO: 711 - RNA, 21 nt, Homo sapiens
cuggauggcuccuccaugucu

SEQ ID NO: 712 - RNA, 20 nt, Homo sapiens
cuguaugcccucaccgcuca

SEQ ID NO: 713 - RNA, 22 nt, Homo sapiens
aucacacaaaggcaacuuuugu

SEQ ID NO: 714 - RNA, 22 nt, Homo sapiens
aaaccguuaccauuacugaguu

SEQ ID NO: 715 - RNA, 22 nt, Homo sapiens
aaguucuguuauacacucaggc

SEQ ID NO: 716 - RNA, 22 nt, Homo sapiens
cagcagcaauucauguuuugaa

SEQ ID NO: 717 - RNA, 21 nt, Homo sapiens
ugucuugcaggccgucaugca

SEQ ID NO: 718 - RNA, 22 nt, Homo sapiens
acccgucccguucguccccgga

SEQ ID NO: 719 - RNA, 22 nt, Homo sapiens
uuuaggauaagcuugacuuuug

SEQ ID NO: 720 - RNA, 23 nt, Homo sapiens
ucauagcccuguacaaugcugcu

(19) United States
(12) Patent Application Publication
(10) Pub. No.: US 2011/0144914 A1
(43) Pub. Date: Jun. 16, 2011

(54) BIOMARKER ASSAY FOR DIAGNOSIS AND CLASSIFICATION OF CARDIOVASCULAR DISEASE

(76) Inventors: Doug Harrington, San Clemente, CA (US); Evangelos Hytopoulos, San Mateo, CA (US); Bruce Phelps, Clayton, CA (US)

(21) Appl. No.: 12/964,719

(22) Filed: Dec. 9, 2010

Related U.S. Application Data
(60) Provisional application No. 61/285,121, filed on Dec. 9, 2009.

(51) Int. Cl. G06F 19/00; G01N 33/48

(52) U.S. Cl. 702/19

(57) ABSTRACT

The disclosed methods, assays and kits identify biomarkers, particularly miRNA and/or protein biomarkers, for assessing the cardiovascular health of a human. In certain embodiments, methods, assays and kits, circulating miRNA and/or protein biomarkers are identified for assessing the cardiovascular health of a human.

### CROSS REFERENCE TO RELATED APPLICATIONS

This application claims priority to U.S. Provisional Patent Application No. 61/285,121, filed on Dec. 9, 2009, which is incorporated by reference herein in its entirety.

### BACKGROUND

Atherosclerotic cardiovascular disease (ASCVD) is the primary cause of morbidity and mortality worldwide. Almost 60% of myocardial infarctions (MIs) occur in people with 0 or 1 risk factor. That is, the majority of people that experience a cardiac event are in the low-intermediate or intermediate risk categories as assessed by current methods.

A combination of genetic and environmental factors is responsible for the initiation and progression of the disease. Atherosclerosis is often asymptomatic and goes undetected by current diagnostic methods. In fact, for many, the first symptom of atherosclerotic cardiovascular disease is heart attack or sudden cardiac death.

An assay and method that can accurately predict and diagnose cardiovascular disease and development is highly desirable.

### BRIEF SUMMARY

The disclosure provides methods, assays and kits for assessing the cardiovascular health of a human. In one embodiment, a method for assessing the cardiovascular health of a human is provided comprising: a) obtaining a biological sample from a human; b) determining levels of at least 2 miRNA markers selected from miRNAs listed in Table 20 in the biological sample; c) obtaining a dataset comprised of the levels of each miRNA marker; d) inputting the data into an analytical classification process that uses the data to classify the biological sample, wherein the classification is selected from the group consisting of an atherosclerotic cardiovascular disease classification, a healthy classification, a medication exposure classification, a no medication exposure classification; and e) determining a treatment regimen for the human based on the classification in step (d); wherein the cardiovascular health of the human is assessed.

A method for assessing the cardiovascular health of a human comprising: a) obtaining a biological sample from a human; b) determining levels of at least 3 protein markers selected from the group consisting of IL-16, sFas, Fas ligand, MCP-3, HGF, CTACK, EOTAXIN, adiponectin, IL-18, TIMP.4, TIMP.1, CRP, VEGF, and EGF in the biological sample; c) obtaining a dataset comprised of the levels of each protein marker; d) inputting the data into an analytical classification process that uses the data to classify the biological sample, wherein the classification is selected from the group consisting of an atherosclerotic cardiovascular disease classification, a healthy classification, a medication exposure classification, a no medication exposure classification; and e) determining a treatment regimen for the human based on the classification in step (d); wherein the cardiovascular health of the human is assessed.

A method for assessing the cardiovascular health of a human to determine the need for or effectiveness of a treatment regimen comprising: obtaining a biological sample from a human; determining levels of at least 2 miRNA markers selected from miRNAs listed in Table 20 in the biological sample; determining levels of at least 3 protein biomarker selected from the group consisting of IL-16, sFas, Fas ligand, MCP-3, HGF, CTACK, EOTAXIN, adiponectin, IL-18, TIMP.4, TIMP.1, CRP, VEGF, and EGF in the biological sample; obtaining a dataset comprised of the individual levels of the miRNA markers and the protein biomarkers; inputting the data into an analytical classification process that uses the data to classify the biological sample, wherein the classification is selected from the group consisting of an atherosclerotic cardiovascular disease classification, a healthy classification, a medication exposure classification, a no medication exposure classification; and classifying the biological sample according to the output of the classification process and determining a treatment regimen for the human based on the classification.

In yet another embodiment, a kit for assessing the cardiovascular health of a human to determine the need for or effectiveness of a treatment regimen is provided. The kit comprises: an assay for determining levels of at least two miRNA markers selected from the miRNAs listed in Table 20 in the biological sample and/or for determining the levels of at least 3 protein markers selected from the group consisting of IL-16, sFas, Fas ligand, MCP-3, HGF, CTACK, EOTAXIN, adiponectin, IL-18, TIMP.4, TIMP.1, CRP, VEGF, and EGF in the biological sample; instructions for (1) obtaining a dataset comprised of the levels of each miRNA and/or protein marker, (2) inputting the data into an analytical classification process that uses the data to classify the biological sample, wherein the classification is selected from the group consisting of an atherosclerotic cardiovascular disease classification, a healthy classification, a medication exposure classification, a no medication exposure classification; (3) and determining a treatment regimen for the human based on the classification.

In yet another embodiment, methods for assessing the risk of a cardiovascular event of a human comprising: a) obtaining a biological sample from a human; b) determining levels of three or more protein biomarkers selected from the group consisting of IL-16, sFas, Fas ligand, MCP-3, HGF, CTACK, EOTAXIN, adiponectin, IL-18, TIMP.4, TIMP.1, CRP, VEGF, and EGF and/or 2 or more of the miRNAs in Table 20 in the sample; c) obtaining a dataset comprised of the levels of each protein and/or miRNA biomarkers; d) inputting the data into a risk prediction analysis process to determine the risk of a cardiovascular event based on the dataset; and e) determining a treatment regimen for the human based on the predicted risk of a cardiovascular event in step (d); wherein the risk of a cardiovascular event of the human is assessed.

### DESCRIPTION OF THE DRAWINGS

FIG. 1 is a graph depicting the expected classification performance for a set of 52 samples (26 cases and 26 controls) based on a logistic regression approach. The expected AUC and corresponding 95% confidence interval was obtained from 500 simulations of classifying sets of 52 either individual or pooled samples. Open circles on error bars represent the expected value and the confidence interval using pooled samples (5 samples in each pool), with a biomarker concentration or score value assumed to follow a log-normal distribution. Open circles on solid error bars represent expected value and confidence interval using individual samples from the same distribution. Solid black dots represent the theoretical result. The x-axis represent differences in the mean for the case and control biomarker or score distribution.

FIG. 2 is a graph depicting the expected classification performance for a set of 52 samples (26 cases and 26 controls) based on a logistic regression approach. The expected AUC and corresponding 95% confidence interval was obtained from 500 simulations of classifying sets of 52 either individual or pooled samples. Open circles on dashed error bars represent the expected value and the confidence interval using pooled samples (5 samples in each pool), with a biomarker concentration or score value assumed to follow a normal distribution. Open circles on solid error bars represent expected value and confidence interval using individual samples from the same distribution. Solid black dots represent the theoretical result. The x-axis represents differences in the mean for the case and control biomarker or score distribution.

FIG. 3 is a graph of the AUC values distribution for the classification of pooled samples based on based on models selecting covariates from a set of 44 miR species. The calculation of the AUC values is based on obtaining 100 prevalidated classification score vectors through fitting penalized logistic regression models (with L1 penalty) to the data. The x-axis represents the AUC and the y-axis represents the frequency. As shown, the average AUC is 0.68.

FIG. 4 is a graph of the AUC values distribution for the classification of individual samples based on models selecting covariates from a set of 44 miR species. The calculation of the AUC values is based on obtaining 100 prevalidated classification score vectors through fitting penalized logistic regression models (with L1 penalty) to the data. As shown, the average AUC is 0.78.

FIG. 5 is a graph of the AUC values distribution for the classification of individual samples based on models selecting covariates from a set of 44 miR species and 47 protein biomarkers. The calculation of the AUC values is based on obtaining 100 prevalidated classification score vectors through fitting penalized logistic regression models (with L1 penalty) to the data. As shown, the average AUC is 0.75.

FIG. 6 is a graph showing distribution of the correlations between miR and protein, including the highest negative correlation and highest positive correlation indicated by the vertical lines.

FIG. 7 is a graph showing the distribution of the correlations between the miRs alone.

FIG. 8 is a graph showing the AUC distribution based on prevalidated score (500 repeats) calculated based on protein biomarker data alone.

FIG. 9 is a graph showing the univariate hazard ratio for the protein biomarkers normalized to the mean and standard deviation of the controls.

FIG. 10 is a graph showing the adjusted hazard ratio (HR) for protein biomarkers. Adjustment was based on traditional risk factors (TRFs): age, gender, systolic blood pressure (BP), diastolic BP, cholesterol, high density lipoprotein (HDL), hypertension, use of hypertension drug, hyperlipidemia, diabetes, and smoking status.

FIGS. 11 A and B are graphs showing the markers with the highest time-dependent AUC and corresponding values for up to 5 years of follow-up. The AUC for sFas, NT.proBNP, MIG, IL.16, MIG, and ANG2 are shown in FIG. 11A and FasLigand, SCD40L, adiponectin, MCP.3, leptin and rantes are shown in FIG. 11B.

FIG. 12 is a graph of the absolute value and standard error of the drop-in-deviance as a function of the number of terms in a Cox proportional Hazard regression model. The optimum number of markers to be included in a model is selected using the 1-standard error rule.

FIGS. 13 A and B are graphs showing the kernel density estimate of the linear predictor obtained from 4 Cox PH models on the Marshfield sample set for controls and cases, respectively.

FIGS. 14 A and B are graphs showing the kernel density estimate of linear predictor obtained from 4 Cox PH models on the MESA sample set for controls and cases, respectively.

### DETAILED DESCRIPTION

The disclosure provides methods, assays and kits for assessing the cardiovascular health of a human, and particularly, to predict, diagnose, and monitor atherosclerotic cardiovascular disease (ASCVD) in a human. The disclosed methods, assays and kits identify circulating micro ribonucleic acid (miRNA) biomarkers and/or protein biomarkers for assessing the cardiovascular health of a human. In certain embodiments of the methods, assays and kits, circulating miRNA and/or protein biomarkers are identified for assessing the cardiovascular health of a human.

In one embodiment, the disclosure provides a method for assessing the cardiovascular health of a human to determine the need for, or effectiveness of, a treatment regimen comprising: obtaining a biological sample from a human; determining levels of at least 2 miRNA markers selected from the group consisting of the list in Table 20 in the biological sample; obtaining a dataset comprised of the levels of each miRNA marker; inputting the data into an analytical classification process that uses the data to classify the biological sample, wherein the classification is selected from the group consisting of an atherosclerotic cardiovascular disease classification, a healthy classification, a medication exposure classification, a no medication exposure classification; and classifying the biological sample according to the output of the classification process and determining a treatment regimen for the human based on the classification.

In certain embodiments, a method for assessing the cardiovascular health of a human to determine the need for, or effectiveness of, a treatment regimen is disclosed comprising: obtaining a biological sample from a human; determining levels of at least 3 protein biomarkers selected from the group consisting of IL-16, sFas, Fas ligand, MCP-3, HGF, CTACK, EOTAXIN, adiponectin, IL-18, TIMP.4, TIMP.1, CRP, VEGF, and EGF in the biological sample; obtaining a dataset comprised of the levels of each protein marker; inputting the data into an analytical classification process that uses the data to classify the biological sample, wherein the classification is selected from the group consisting of an atherosclerotic cardiovascular disease classification, a healthy classification, a medication exposure classification, a no medication exposure classification; and classifying the biological sample according to the output of the classification process and determining a treatment regimen for the human based on the classification.

In another embodiment, a method is provided for assessing the cardiovascular health of a human. In certain embodiments, the assessment can be used to determine the need for or effectiveness of a treatment regimen. The method comprises: obtaining a biological sample from a human; determining levels of at least two miRNA markers selected from the miRNAs listed in Table 20 in the biological sample; determining levels of at least three protein biomarker selected from the group consisting of IL-16, sFas, Fas ligand, MCP-3, HGF, CTACK, EOTAXIN, adiponectin, IL-18, TIMP.4, TIMP.1, CRP, VEGF, and EGF in the biological sample; obtaining a dataset comprised of the levels of the individual miRNA markers and the protein biomarkers; inputting the data into an analytical classification process that uses the data to classify the biological sample, wherein the classification is selected from the group consisting of an atherosclerotic cardiovascular disease classification, a healthy classification, a medication exposure classification, a no medication exposure classification; and classifying the biological sample according to the output of the classification process and determining a treatment regimen for the human based on the classification.

In yet another embodiment, methods for assessing the risk of a cardiovascular event of a human. The method comprises obtaining a biological sample from a human; and determining the levels of (1) three or more protein biomarkers selected from the group consisting of IL-16, sFas, Fas ligand, MCP-3, HGF, CTACK, EOTAXIN, adiponectin, IL-18, TIMP.4, TIMP.1, CRP, VEGF, and EGF and/or (2) two or more of the miRNAs in Table 20 in the sample. In the method, a dataset is obtained comprised of the levels of each protein and/or miRNA biomarkers. The data is input into a risk prediction analysis process to predict the risk of a cardiovascular event based on the dataset; and a treatment regimen can be determined for the human based on the predicted risk of a cardiovascular event. The risk of a cardiovascular even can be predicted for about 1 year, about 2 years, about 3 years, about 4 years, about 5 years or more from the date on which the sample is obtained and/or analyzed. The predicted cardiovascular event, as described below, can be development of atherosclerotic disease, a MI, etc.

The terms “marker” and “biomarker” are used interchangeably throughout the disclosure.

In the disclosed methods, the number of miRNA markers that are detected and whose levels are determined, can be 1, or more than 1, such as 2, 3, 4, 5, 6, 7, 8, 9, 10, or more. In certain embodiments, the number of miRNA markers detected is 3, or 5, or more. The number of protein biomarkers that are detected, and whose levels are determined, can be 1, or more than 1, such as 2, 3, 4, 5, 6, 7, 8, 9, 10, or more. In certain embodiments, 1, 2, 3, or 5 or more miRNA markers are detected and levels are determined and 1, 2, 3, or 5 or more protein biomarkers are detected and levels are determined.

The methods of this disclosure are useful for diagnosing and monitoring atherosclerotic disease. Atherosclerotic disease is also known as atherosclerosis, arteriosclerosis, atheromatous vascular disease, arterial occlusive disease, or cardiovascular disease, and is characterized by plaque accumulation on vessel walls and vascular inflammation. Vascular inflammation is a hallmark of active atherosclerotic disease, unstable plaque, or vulnerable plaque. The plaque consists of accumulated intracellular and extracellular lipids, smooth muscle cells, connective tissue, inflammatory cells, and glycosaminoglycans. Certain plaques also contain calcium. Unstable or active or vulnerable plaques are enriched with inflammatory cells.

By way of example, the present disclosure includes methods for generating a result useful in diagnosing and monitoring atherosclerotic disease by obtaining a dataset associated with a sample, where the dataset at least includes quantitative data about miRNA markers alone or in combination with protein biomarkers which have been identified as predictive of atherosclerotic disease, and inputting the dataset into an analytic process that uses the dataset to generate a result useful in diagnosing and monitoring atherosclerotic disease. This quantitative data can include DNA, RNA, protein expression levels, and a combination thereof.

The methods, assays and kits disclosed are also useful for diagnosing and monitoring complications of cardiovascular disease, including myocardial infarction (MI), acute coronary syndrome, stroke, heart failure, and angina. An example of a common complication is MI, which refers to ischemic myocardial necrosis usually resulting from abrupt reduction in coronary blood flow to a segment of myocardium. In the great majority of patients with acute MI, an acute thrombus, often associated with plaque rupture, occludes the artery that supplies the damaged area. Plaque rupture occurs generally in arteries previously partially obstructed by an atherosclerotic plaque enriched in inflammatory cells. Another example of a common atherosclerotic complication is angina, a condition with symptoms of chest pain or discomfort resulting from inadequate blood flow to the heart.

The present disclosure identifies profiles of biomarkers of inflammation that can be used for diagnosis and classification of atherosclerotic cardiovascular disease as well as prediction of the risk of a cardiovascular event (e.g., MI) within a specific period of time from blood draw for a given individual. The miRNA and protein biomarkers assayed in the present disclosure are those identified using a learning algorithm as being capable of distinguishing between different atherosclerotic classifications, e.g., diagnosis, staging, prognosis, monitoring, therapeutic response, and prediction of pseudo-coronary calcium score. Other data useful for making atherosclerotic classifications, such as clinical indicia (e.g., traditional risk factors) may also be a part of a dataset used to generate a result useful for atherosclerotic classification.

Datasets containing quantitative data for the various miRNA and protein biomarkers markers disclosed herein, alone or in combination, and quantitative data for other dataset components (e.g., DNA, RNA, measures of clinical indicia) can be input into an analytical process and used to generate a result. The analytic process may be any type of learning algorithm with defined parameters, or in other words, a predictive model. Predictive models can be developed for a variety of atherosclerotic classifications or risk prediction by applying learning algorithms to the appropriate type of reference or control data. The result of the analytical process/predictive model can be used by an appropriate individual to take the appropriate course of action. For example, if the classification is “healthy” or “atherosclerotic cardiovascular disease”, then a result can be used to determine the appropriate clinical course of treatment for an individual.

MicroRNA (also referred to herein as miRNA, μRNA, mi-R) is a form of single-stranded RNA molecule of about 17-27 nucleotides in length, which regulates gene expression. miRNAs are encoded by genes from whose DNA they are transcribed but miRNAs are not translated into protein (i.e. they are non-coding RNAs); instead each primary transcript (a pri-miRNA) is processed into a short stem-loop structure called a pre-miRNA and finally into a functional miRNA.

miRNA markers associated with inflammation and useful for assessing the cardiovascular health of a human include, but are not limited to, one or more of miR-26a, miR-16, miR-222, miR-10b, miR-93, miR-192, miR-15a, miR-125-a.5p, miR-130a, miR-92a, miR-378, miR-20a, miR-20b, miR-107, miR-186, hsa.let.7f, miR-19a, miR-150, miR-106b, miR-30c, and let 7b. In certain embodiments, the miRNA markers include one or more of miR-26a, miR-16, miR-222, miR-10b, miR-93, miR-192, miR-15a, miR-125-a.5p, miR-130a, miR-92a, miR-378, and let 7b. In particular, the miRNAs listed in Table 20 are useful in assessing cardiovascular health of a human.

Protein biomarkers associated with inflammation and useful for assessing the cardiovascular health of a human include, but are not limited to, one or more of RANTES, TIMP1, MCP-1, MCP-2, MCP-3, MCP-4, eotaxin, IP-10, M-CSF, IL-3, TNFa, Ang-2, IL-5, IL-7, IGF-1, sVCAM, sICAM-1, E-selectin, P-selection, interleukin-6, interleukin-18, creatine kinase, LDL, oxLDL, LDL particle size, Lipoprotein(a), troponin I, troponin T, LPPLA2, CRP, HDL, triglycerides, insulin, BNP, fractalkine, osteopontin, osteoprotegerin, oncostatin-M, Myeloperoxidase, ADMA, PAI-1 (plasminogen activator inhibitor), SAA (circulating amyloid A), t-PA (tissue-type plasminogen activator), sCD40 ligand, fibrinogen, homocysteine, D-dimer, leukocyte count, heart-type fatty acid binding protein, MMP1, plasminogen, folate, vitamin B6, leptin, soluble thrombomodulin, PAPPA, MMP9, MMP2, VEGF, PIGF, HGF, vWF, and cystatin C. In certain embodiments, the protein biomarkers include one or more of IL-16, sFas, Fas ligand, MCP-3, HGF, CTACK, EOTAXIN, adiponectin, IL-18, TIMP.4, TIMP.1, CRP, VEGF, and EGF. In addition to the specific biomarkers, the disclosure further includes biomarker variants that are about 90%, about 95%, or about 97% identical to the exemplified sequences. Variants, as used herein, include polymorphisms, splice variants, mutations, and the like.

Protein biomarkers can be detected in a variety of ways. For example, in vivo imaging may be utilized to detect the presence of atherosclerosis-associated proteins in heart tissue. Such methods may utilize, for example, labeled antibodies or ligands specific for such proteins. In these embodiments, a detectably-labeled moiety, e.g., an antibody, ligand, etc., which is specific for the polypeptide is administered to an individual (e.g., by injection), and labeled cells are located using standard imaging techniques, including, but not limited to, magnetic resonance imaging, computed tomography scanning, and the like. Detection may utilize one, or a cocktail of, imaging reagents.

Additional markers can be selected from one or more clinical indicia, including but not limited to, age, gender, LDL concentration, HDL concentration, triglyceride concentration, blood pressure, body mass index, CRP concentration, coronary calcium score, waist circumference, tobacco smoking status, previous history of cardiovascular disease, family history of cardiovascular disease, heart rate, fasting insulin concentration, fasting glucose concentration, diabetes status, and use of high blood pressure medication. Additional clinical indicia useful for making atherosclerotic classifications can be identified using learning algorithms known in the art, such as linear discriminant analysis, support vector machine classification, recursive feature elimination, prediction analysis of microarray, logistic regression, CART, FlexTree, LART, random forest, MART, and/or survival analysis regression, which are known to those of skill in the art and are further described herein.

The analytical classification disclosed herein, can comprise the use of a predictive model. The predictive model further comprises a quality metric of at least about 0.68 or higher for classification. In certain embodiments, the quality metric is at least about 0.70 or higher for classification. In certain embodiments, the quality metric is selected from area under the curve (AUC), hazard ratio (HR), relative risk (RR), reclassification, positive predictive value (PPV), negative predictive value (NPV), accuracy, sensitivity and specificity, Net reclassification Index, Clinical Net reclassification Index. These and other metrics can be used as described herein. Further, various terms can be selected to provide a quality metric.

Quantitative data is obtained for each component of the dataset and input into an analytic process with previously defined parameters (the predictive model) and then used to generate a result.

The data may be obtained via any technique that results in an individual receiving data associated with a sample. For example, an individual may obtain the dataset by generating the dataset himself by methods known to those in the art. Alternatively, the dataset may be obtained by receiving a dataset or one or more data values from another individual or entity. For example, a laboratory professional may generate certain data values while another individual, such as a medical professional, may input all or part of the dataset into an analytic process to generate the result.

One of skill should understand that although reference is made to “a sample” throughout the disclosure that the quantitative data may be obtained from multiple samples varying in any number of characteristics, such as the method of procurement, time of procurement, tissue origin, etc.

In methods of generating a result useful for atherosclerotic classification, the expression pattern in blood, serum, etc. of the protein markers provided herein is obtained. The quantitative data associated with the protein markers of interest can be any data that allows generation of a result useful for atherosclerotic classification, including measurement of DNA or RNA levels associated with the markers but is typically protein expression patterns. Protein levels can be measured via any method known to those of skill in the art that generates a quantitative measurement either individually or via high-throughput methods as part of an expression profile. For example, a blood-derived patient sample, e.g., blood, plasma, serum, etc. may be applied to a specific binding agent or panel of specific binding agents to determine the presence and quantity of the protein markers of interest.

Blood samples, or samples derived from blood, e.g. plasma, serum, etc. are assayed for the presence of expression levels of the miRNA markers alone or in combination with protein markers of interest. Typically a blood sample is drawn, and a derivative product, such as plasma or serum, is tested. In addition, the sample can be derived from other bodily fluids such as saliva, urine, semen, milk or sweat. Samples can further be derived from tissue, such as from a blood vessel, such as an artery, vein, capillary and the like. Further, when both miRNA and protein biomarkers are assayed, they can be derived from the same or different samples. That is, for example, an miRNA biomarker can be assayed in a blood derived sample and a protein biomarker can be assayed in a tissue sample.

The quantitative data associated with the miRNA and protein markers of interest typically takes the form of an expression profile. Expression profiles constitute a set of relative or absolute expression values for a number of miRNA or protein products corresponding to the plurality of markers evaluated. In various embodiments, expression profiles containing expression patterns at least about 2, 3, 4, 5, 6, 7 or more markers are produced. The expression pattern for each differentially expressed component member of the expression profile may provide a particular specificity and sensitivity with respect to predictive value, e.g., for diagnosis, prognosis, monitoring treatment, etc.

Numerous methods for obtaining expression data are known, and any one or more of these techniques, singly or in combination, are suitable for determining expression patterns and profiles in the context of the present disclosure.

For example, DNA and RNA (mRNA, pri-miRNA, pre-miRNA, miRNA, precursor hairpin RNA, microRNP, and the like) expression patterns can be evaluated by northern analysis, PCR, RT-PCR, Taq Man analysis, FRET detection, monitoring one or more molecular beacon, hybridization to an oligonucleotide array, hybridization to a cDNA array, hybridization to a polynucleotide array, hybridization to a liquid microarray, hybridization to a microelectric array, cDNA sequencing, clone hybridization, cDNA fragment fingerprinting, serial analysis of gene expression (SAGE), subtractive hybridization, differential display and/or differential screening. These and other techniques are well known to those of skill in the art.

The present disclosure includes nucleic acid molecules, preferably in isolated form. As used herein, a nucleic acid molecule is to be “isolated” when the nucleic acid molecule is substantially separated from contaminant nucleic acid molecules encoding other polypeptides. The term “nucleic acid” is defined as coding and noncoding RNA or DNA. Nucleic acids that are complementary to, that is, hybridize to, and remain stably bound to the molecules under appropriate stringency conditions are included within the scope of this disclosure. Such sequences exhibit at least 50%, 60%, 70% or 75%, preferably at least about 80-90%, more preferably at least about 92-94%, and even more preferably at least about 95%, 98%, 99% or more nucleotide sequence identity with the RNAs disclosed herein, and include insertions, deletions, wobble bases, substitutions and the like. Further contemplated are sequences sharing at least about 50%, 60%, 70% or 75%, preferably at least about 80-90%, more preferably at least about 92-94%, and most preferably at least about 95%, 98%, 99% or more identity with the protein biomarker sequences disclosed herein

Specifically contemplated within the scope of the disclosure are genomic DNA, cDNA, RNA (mRNA, pri-miRNA, pre-miRNA, miRNA, hairpin precursor RNA, RNP, etc.) molecules, as well as nucleic acids based on alternative backbones or including alternative bases, whether derived from natural sources or synthesized.

Homology or identity at the nucleotide or amino acid sequence level is determined by BLAST (Basic Local Alignment Search Tool) analysis using the algorithm employed by the programs blastp, blastn, blastx, tblastn and tblastx which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a preselected threshold of significance. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix, recommended for query sequences over 85 nucleotides or amino acids in length.

For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters were adjusted as follows: Q=10 (gap creation penalty); R=10 (gap extension penalty); wink=1 (generates word hits at every winkth position along the query); and gapw-16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

The present disclosure further provides fragments of the disclosed nucleic acid molecules. As used herein, a fragment of a nucleic acid molecule refers to a small portion of the coding or non-coding sequence. The size of the fragment will be determined by the intended use. For example, if the fragment is chosen so as to encode an active portion of the protein, the fragment will need to be large enough to encode the functional region(s) of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming.

Protein expression patterns can be evaluated by any method known to those of skill in the art which provides a quantitative measure and is suitable for evaluation of multiple markers extracted from samples such as one or more of the following methods: ELISA sandwich assays, flow cytometry, mass spectrometric detection, calorimetric assays, binding to a protein array (e.g., antibody array), or fluorescent activated cell sorting (FACS).

In one embodiment, an approach involves the use of labeled affinity reagents (e.g., antibodies, small molecules, etc.) that recognize epitopes of one or more protein products in an ELISA, antibody-labelled fluorescent bead array, antibody array, or FACS screen. Methods for producing and evaluating antibodies are well known in the art.

A number of suitable high throughput formats exist for evaluating expression patterns and profiles of the disclosed biomarkers. Typically, the term high throughput refers to a format that performs at least about 100 assays, or at least about 500 assays, or at least about 1000 assays, or at least about 5000 assays, or at least about 10,000 assays, or more per day. When enumerating assays, either the number of samples or the number of markers assayed can be considered.

Numerous technological platforms for performing high throughput expression analysis are known. Generally, such methods involve a logical or physical array of either the subject samples, or the protein markers, or both. Common array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384 or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis. Exemplary systems include, e.g., xMAP® technology from Luminex (Austin, Tex.), the SECTOR® Imager with MULTI-ARRAY® and MULTI-SPOT® technologies from Meso Scale Discovery (Gaithersburg, Md.), the ORCA™ system from Beckman-Coulter, Inc. (Fullerton, Calif.) and the ZYMATE™ systems from Zymark Corporation (Hopkinton, Mass.), miRCURY LNA™ microRNA Arrays (Exiqon, Woburn, Mass.).

Alternatively, a variety of solid phase arrays can favorably be employed to determine expression patterns in the context of the disclosed methods, assays and kits. Exemplary formats include membrane or filter arrays (e.g., nitrocellulose, nylon), pin arrays, and bead arrays (e.g., in a liquid “slurry”). Typically, probes corresponding to nucleic acid or protein reagents that specifically interact with (e.g., hybridize to or bind to) an expression product corresponding to a, member of the candidate library, are immobilized, for example by direct or indirect cross-linking, to the solid support. Essentially any solid support capable of withstanding the reagents and conditions necessary for performing the particular expression assay can be utilized. For example, functionalized glass, silicon, silicon dioxide, modified silicon, any of a variety of polymers, such as (poly)tetrafluoroethylene, (poly)vinylidenedifluoride, polystyrene, polycarbonate, or combinations thereof can all serve as the substrate for a solid phase array.

In one embodiment, the array is a “chip” composed, e.g., of one of the above-specified materials. Polynucleotide probes, e.g., RNA or DNA, such as cDNA, synthetic oligonucleotides, and the like, or binding proteins such as antibodies or antigen-binding fragments or derivatives thereof, that specifically interact with expression products of individual components of the candidate library are affixed to the chip in a logically ordered manner, i.e., in an array. In addition, any molecule with a specific affinity for either the sense or anti-sense sequence of the marker nucleotide sequence (depending on the design of the sample labeling), can be fixed to the array surface without loss of specific affinity for the marker and can be obtained and produced for array production, for example, proteins that specifically recognize the specific nucleic acid sequence of the marker, ribozymes, peptide nucleic acids (PNA), or other chemicals or molecules with specific affinity.

Microarray expression may be detected by scanning the microarray with a variety of laser or CCD-based scanners, and extracting features with numerous software packages, for example, IMAGENE™ (Biodiscovery), Feature Extraction Software (Agilent), SCANLYZE™ (Stanford Univ., Stanford, Calif.), GENEPIX™ (Axon Instruments).

High-throughput protein systems include commercially available systems from Ciphergen Biosystems, Inc. (Fremont, Calif.) such as PROTEIN CHIP™ arrays, and FASTQUANT™ human chemokine protein microspot array (S&S Bioscences Inc., Keene, N.H., US).

Quantitative data regarding other dataset components, such as clinical indicia, metabolic measures, and genetic assays, can be determined via methods known to those of skill in the art.

The quantitative data thus obtained about the miRNA, protein markers and other dataset components (i.e., clinical indicia and the like) is subjected to an analytic process with parameters previously determined using a learning algorithm, i.e., inputted into a predictive model. The parameters of the analytic process may be those disclosed herein or those derived using the guidelines described herein. Learning algorithms such as linear discriminant analysis, recursive feature elimination, a prediction analysis of microarray, logistic regression, CART, FlexTree, LART, random forest, MART, or another machine learning algorithm are applied to the appropriate reference or training data to determine the parameters for analytical processes suitable for a variety of atherosclerotic classifications.

The analytic process used to generate a result (classification, survival/time-to-event, etc.) may be any type of process capable of providing a result useful for classifying a sample, for example, comparison of the obtained dataset with a reference dataset, a linear algorithm, a quadratic algorithm, a decision tree algorithm, or a voting algorithm.

Various analytic processes for obtaining a result useful for making an atherosclerotic classification are described herein, however, one of skill in the art will readily understand that any suitable type of analytic process is within the scope of this disclosure.

Prior to input into the analytical process, the data in each dataset is collected by measuring the values for each marker, usually in duplicate or triplicate or in multiple replicates. The data may be manipulated, for example, raw data may be transformed using standard curves, and the average of replicate measurements used to calculate the average and standard deviation for each patient. These values may be transformed before being used in the models, e.g. log-transformed, Box-Cox transformed, etc. This data can then be input into the analytical process with defined parameters.

The analytic process may set a threshold for determining the probability that a sample belongs to a given class. The probability preferably is at least 50%, or at least 60% or at least 70% or at least 80%, at least 90%, or higher.

In other embodiments, the analytic process determines whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

In general, the analytical process will be in the form of a model generated by a statistical analytical method such as those described below. Examples of such analytical processes may include a linear algorithm, a quadratic algorithm, a polynomial algorithm, a decision tree algorithm, a voting algorithm. A linear algorithm may have the form:

$$R = {C}_{0} + {\sum }_{i = 1}^{N} {C}_{i} {x}_{i}$$

where R is the useful result obtained. C₀ is a constant that may be zero. Cᵢ and xᵢ are the constants and the value of the applicable biomarker or clinical indicia, respectively, and N is the total number of markers.

A quadratic algorithm may have the form:

$$R = {C}_{0} + {\sum }_{i = 1}^{N} {C}_{i} {x}_{i}^{2}$$

where R is the useful result obtained. C₀ is a constant that may be zero. Cᵢ and xᵢ are the constants and the value of the applicable biomarker or clinical indicia, respectively, and N is the total number of markers.

A polynomial algorithm is a more generalized form of a linear or quadratic algorithm that may have the form:

$$R = {C}_{0} + {\sum }_{i = 0}^{N} {C}_{i} {x}_{i}^{{y}_{i}}$$

where R is the useful result obtained. C₀ is a constant that may be zero. Cᵢ and xᵢ are the constants and the value of the applicable biomarker or clinical indicia, respectively; yᵢ is the power to which xᵢ is raised and N is the total number of markers.

Using any suitable learning algorithm, an appropriate reference or training dataset can be used to determine the parameters of the analytical process to be used for classification, i.e., develop a predictive model. The reference or training dataset to be used will depend on the desired atherosclerotic classification to be determined. The dataset may include data from two, three, four or more classes. For example, to use a supervised learning algorithm to determine the parameters for an analytic process used to diagnose atherosclerosis, a dataset comprising control and diseased samples is used as a training set. Alternatively, if a supervised learning algorithm is to be used to develop a predictive model for atherosclerotic staging, then the training set may include data for each of the various stages of cardiovascular disease.

The following are examples of the types of statistical analysis methods that are available to one of skill in the art to aid in the practice of the disclosed methods, assays and kits. The statistical analysis may be applied for one or both of two tasks. First, these and other statistical methods may be used to identify preferred subsets of markers and other indicia that will form a preferred dataset. In addition, these and other statistical methods may be used to generate the analytical process that will be used with the dataset to generate the result. Several of statistical methods presented herein or otherwise available in the art will perform both of these tasks and yield a model that is suitable for use as an analytical process for the practice of the methods disclosed herein.

Biomarkers whose corresponding features values (e.g., concentration, expression level) are capable of discriminating between, e.g., healthy and atherosclerotic, are identified herein. The identity of these markers and their corresponding features (e.g., concentration, expression level) can be used to develop an analytical process, or plurality of analytical processes, that discriminate between classes of patients. The examples below illustrate how data analysis algorithms can be used to construct a number of such analytical processes. Each of the data analysis algorithms described in the examples use features (e.g., expression values) of a subset of the markers identified herein across a training population that includes healthy and atherosclerotic patients. Specific data analysis algorithms for building an analytical process, or plurality of analytical processes, that discriminate between subjects disclosed herein will be described in the subsections below. Once an analytical process has been built using these exemplary data analysis algorithms or other techniques known in the art, the analytical process can be used to classify a test subject into one of the two or more phenotypic classes (e.g. a healthy or atherosclerotic patient) and/or predict survival/time-to-event. This is accomplished by applying one or more analytical processes to one or more marker profile(s) obtained from the test subject. Such analytical processes, therefore, have enormous value as diagnostic indicators.

The disclosed methods, assays and kits provide, in one aspect, for the evaluation of one or more marker profile(s) from a test subject to marker profiles obtained from a training population. In some embodiments, each marker profile obtained from subjects in the training population, as well as the test subject, comprises a feature for each of a plurality of different markers. In some embodiments, this comparison is accomplished by (i) developing an analytical process using the marker profiles from the training population and (ii) applying the analytical process to the marker profile from the test subject. As such, the analytical process applied in some embodiments of the methods disclosed herein is used to determine whether a test subject has atherosclerosis. In alternate embodiments, the methods disclosed herein determine whether or not a subject will experience a MI, and/or can predict time-to-event (e.g. MI and/or survival).

In some embodiments of the methods disclosed herein, when the results of the application of an analytical process indicate that the subject will likely experience a MI, the subject is diagnosed/classified as a “MI” subject. Alternately, if, for example, the results of the analytical process indicate that a subject will likely develop atherosclerosis, the subject is diagnosed as an “atherosclerotic” subject. If the results of an application of an analytical process indicate that the subject will not develop atherosclerosis, the subject is diagnosed as a healthy subject. Thus, in some embodiments, the result in the above-described binary decision situation has four possible outcomes: (i) truly atherosclerotic, where the analytical process indicates that the subject will develop atherosclerosis and the subject does in fact develop atherosclerosis during the definite time period (true positive, TP); (ii) falsely atherosclerotic, where the analytical process indicates that the subject will develop atherosclerosis and the subject, in fact, does not develop atherosclerosis during the definite time period (false positive, FP); (iii) truly healthy, where the analytical process indicates that the subject will not develop atherosclerosis and the subject, in fact, does not develop atherosclerosis during the definite time period (true negative, TN); or (iv) falsely healthy, where the analytical process indicates that the subject will not develop atherosclerosis and the subject, in fact, does develop atherosclerosis during the definite time period (false negative, FN).

It will be appreciated that other definitions for TP, FP, TN, FN can be made. While all such alternative definitions are within the scope of the disclosed methods, assays and kits, for ease of understanding, the definitions for TP, FP, TN, and FN given by definitions (i) through (iv) above will be used herein, unless otherwise stated.

As will be appreciated by those of skill in the art, a number of quantitative criteria can be used to communicate the performance of the comparisons made between a test marker profile and reference marker profiles (e.g., the application of an analytical process to the marker profile from a test subject). These include positive predicted value (PPV), negative predicted value (NPV), specificity, sensitivity, accuracy, and certainty. In addition, other constructs such a receiver operator curves (ROC) can be used to evaluate analytical process performance. As used herein: PPV=TP/(TP+FP), NPV=TN/(TN+FN), specificity=TN/(TN+FP), sensitivity=TP/(TP+FN), and accuracy=certainty=(TP+TN)/N.

Here, N is the number of samples compared (e.g., the number of test samples for which a determination of atherosclerotic or healthy is sought). For example, consider the case in which there are ten subjects for which this classification is sought. Marker profiles are constructed for each of the ten test subjects. Then, each of the marker profiles is evaluated by applying an analytical process, where the analytical process was developed based upon marker profiles obtained from a training population. In this example, N, from the above equations, is equal to 10. Typically, N is a number of samples, where each sample was collected from a different member of a population. This population can, in fact, be of two different types. In one type, the population comprises subjects whose samples and phenotypic data (e.g., feature values of markers and an indication of whether or not the subject developed atherosclerosis) was used to construct or refine an analytical process. Such a population is referred to herein as a training population. In the other type, the population comprises subjects that were not used to construct the analytical process. Such a population is referred to herein as a validation population. Unless otherwise stated, the population represented by N is either exclusively a training population or exclusively a validation population, as opposed to a mixture of the two population types. It will be appreciated that scores such as accuracy will be higher (closer to unity) when they are based on a training population as opposed to a validation population. Nevertheless, unless otherwise explicitly stated herein, all criteria used to assess the performance of an analytical process (or other forms of evaluation of a biomarker profile from a test subject) including certainty (accuracy) refer to criteria that were measured by applying the analytical process corresponding to the criteria to either a training population or a validation population.

In some embodiments, N is more than 1, more than 5, more than 10, more than 20, between 10 and 100, more than 100, or less than 1000 subjects. An analytical process (or other forms of comparison) can have at least about 99% certainty, or even more, in some embodiments, against a training population or a validation population. In other embodiments, the certainty is at least about 97%, at least about 95%, at least about 90%, at least about 85%, at least about 80%, at least about 75%, at least about 70%, at least about 65%, or at least about 60% against a training population or a validation population. The useful degree of certainty may vary, depending on the particular method. As used herein, “certainty” means “accuracy.” In one embodiment, the sensitivity and/or specificity is at least about 97%, at least about 95%, at least about 90%, at least about 85%, at least about 80%, at least about 75%, or at least about 70% against a training population or a validation population. In some embodiments, such analytical processes are used to predict the development of atherosclerosis with the stated accuracy. In some embodiments, such analytical processes are used to diagnoses atherosclerosis with the stated accuracy. In some embodiments, such analytical processes are used to determine a stage of atherosclerosis with the stated accuracy.

The number of features that may be used by an analytical process to classify a test subject with adequate certainty is 2 or more. In some embodiments, it is 3 or more, 4 or more, 10 or more, or between 10 and 200. Depending on the degree of certainty sought, however, the number of features used in an analytical process can be more or less, but in all cases is at least 2. In one embodiment, the number of features that may be used by an analytical process to classify a test subject is optimized to allow a classification of a test subject with high certainty.

In certain embodiments, analytical processes are utilized to predict survival. Survival analyses involve modeling time-to-event data. Proportional hazards models are a class of survival models in statistics. Survival models relate the time that passes before some event occurs to one or more covariates that may be associated with that quantity. In a proportional hazards model, the unique effect of a unit increase in a covariate is multiplicative with respect to the hazard rate. Survival models can be viewed as consisting of two parts: the underlying hazard function, often denoted Λ0(t), describing how the hazard (risk) changes over time at baseline levels of covariates; and the effect parameters, describing how the hazard varies in response to explanatory covariates. A typical medical example would include covariates such as treatment assignment, as well as patient characteristics such as age, gender, and the presence of other diseases in order to reduce variability and/or control for confounding.

The proportional hazards assumption is the assumption that covariates multiply hazard. In the simplest case of stationary coefficients, for example, a treatment with a drug may, say, halve a subject's hazard at any given time t, while the baseline hazard may vary. Note however, that the covariate is not restricted to binary predictors; in the case of a continuous covariate x, the hazard responds logarithmically; each unit increase in x results in proportional scaling of the hazard. Typically under the fully-general Cox model, the baseline hazard is “integrated out”, or heuristically removed from consideration, and the remaining partial likelihood is maximized. The effect of covariates estimated by any proportional hazards model can thus be reported as hazard ratios. The Cox model assumes that if the proportional hazards assumption holds, it is possible to estimate the effect parameters without consideration of the hazard function.

Relevant data analysis algorithms for developing an analytical process include, but are not limited to, discriminant analysis including linear, logistic, and more flexible discrimination techniques; tree-based algorithms such as classification and regression trees (CART) and variants; generalized additive models; neural networks, penalized regression methods, and the like.

In one embodiment, comparison of a test subject's marker profile to a marker profile(s) obtained from a training population is performed, and comprises applying an analytical process. The analytical process is constructed using a data analysis algorithm, such as a computer pattern recognition algorithm. Other suitable data analysis algorithms for constructing analytical process include, but are not limited to, logistic regression or a nonparametric algorithm that detects differences in the distribution of feature values (e.g., a Wilcoxon Signed Rank Test (unadjusted and adjusted)). The analytical process can be based upon 2, 3, 4, 5, 10, 20 or more features, corresponding to measured observables from 1, 2, 3, 4, 5, 10, 20 or more markers. In one embodiment, the analytical process is based on hundreds of features or more. An analytical process may also be built using a classification tree algorithm. For example, each marker profile from a training population can comprise at least 3 features, where the features are predictors in a classification tree algorithm. The analytical process predicts membership within a population (or class) with an accuracy of at least about 70%, at least about 75%, at least about 80%, at least about 85%, at least about 90%, at least about 95%, at least about 97%, at least about 98%, at least about 99%, or about 100%.

Suitable data analysis algorithms are known in the art. In one embodiment, a data analysis algorithm of the disclosure comprises Classification and Regression Tree (CART), Multiple Additive Regression Tree (MART), Prediction Analysis for Microarrays (PAM), or Random Forest analysis. Such algorithms classify complex spectra from biological materials, such as a blood sample, to distinguish subjects as normal or as possessing biomarker levels characteristic of a particular disease state. In other embodiments, a data analysis algorithm of the disclosure comprises ANOVA and nonparametric equivalents, linear discriminant analysis, logistic regression analysis, nearest neighbor classifier analysis, neural networks, principal component analysis, quadratic discriminant analysis, regression classifiers and support vector machines. While such algorithms may be used to construct an analytical process and/or increase the speed and efficiency of the application of the analytical process and to avoid investigator bias, one of ordinary skill in the art will realize that computer-based algorithms are not required to carry out the methods of the present disclosure.

Analytical processes can be used to evaluate biomarker profiles, regardless of the method that was used to generate the marker profile. For example, suitable analytical processes can be used to evaluate marker profiles generated using gas chromatography, spectra obtained by static time-of-flight secondary ion mass spectrometry (TOF-SIMS), distinguishing between bacterial strains with high certainty (79-89% correct classification rates) by analysis of MALDI-TOF-MS spectra, use of MALDI-TOF-MS and liquid chromatography-electrospray ionization mass spectrometry (LC/ESI-MS) to classify profiles of biomarkers in complex biological samples.

One approach to developing an analytical process using expression levels of markers disclosed herein is the nearest centroid classifier. Such a technique computes, for each class (e.g., healthy and atherosclerotic), a centroid given by the average expression levels of the markers in the class, and then assigns new samples to the class whose centroid is nearest. This approach is similar to k-means clustering except clusters are replaced by known classes. This algorithm can be sensitive to noise when a large number of markers are used. One enhancement to the technique uses shrinkage: for each marker, differences between class centroids are set to zero if they are deemed likely to be due to chance. This approach is implemented in the Prediction Analysis of Microarray, or PAM. Shrinkage is controlled by a threshold below which differences are considered noise. Markers that show no difference above the noise level are removed. A threshold can be chosen by cross-validation. As the threshold is decreased, more markers are included and estimated classification errors decrease, until they reach a bottom and start climbing again as a result of noise markers—a phenomenon known as overfitting.

Multiple additive regression trees (MART) represent another way to construct an analytical process that can be used in the methods disclosed herein. A generic algorithm for MART is:

### 1. Initialize

$${F}_{0} ( x ) = arg min y {\sum }_{i = 1}^{N} L ( {y}_{i} , y )$$

2. For m=I to M:

(a) For I=1, 2, . . . , N compute

$${r}_{im} = - \vert \frac{\partial L ( {y}_{i} , f ( {x}_{i} ) )}{\partial f ( {x}_{i} )} \vert j - {j}_{m} - 1$$

(b) Fit a regression tree to the targets rim giving terminal regions Rjm, j=1, 2, . . . Jm

(c) For j=1, 2, . . . Jm compute

$${γ}_{\text{?}} = arg min γ {\sum }_{\text{?}} L ( {y}_{i} , {f}_{m - 1} ( {x}_{i} ) + y )$$

$$( d ) Update fm ( x ) = fm - \text{?} + {\sum }_{\text{?}}^{\text{?}} \text{?}$$

$$\text{?} \text{indicates text missing or illegible when filed}$$

3. Output f(x)=f_{M}(x).

Specific algorithms are obtained by inserting different loss criteria L(y,f(x)). The first line of the algorithm initializes to the optimal constant model, which is just a single terminal node tree. The components of the negative gradient computed in line 2(a) are referred to as generalized pseudo residuals, r. Gradients for commonly used loss functions are known in the art. Tuning parameters associated with the MART procedure are the number of iterations M and the sizes of each of the constituent trees J.sub.m, m=1, 2, . . . , M.

In some embodiments, an analytical process used to classify subjects is built using regression. In such embodiments, the analytical process can be characterized as a regression classifier, preferably a logistic regression classifier. Such a regression classifier includes a coefficient for each of the markers (e.g., the expression level for each such marker) used to construct the classifier. In such embodiments, the coefficients for the regression classifier are computed using, for example, a maximum likelihood approach. In such a computation, the features for the biomarkers (e.g., RT-PCR, microarray data) are used. In certain embodiments, molecular marker data from only two trait subgroups is used (e.g., healthy patients and atherosclerotic patients) and the dependent variable is absence or presence of a particular trait in the subjects for which marker data is available.

In another embodiment, the training population comprises a plurality of trait subgroups (e.g., three or more trait subgroups, four or more specific trait subgroups, etc.). These multiple trait subgroups can correspond to discrete stages in the phenotypic progression from healthy, to mild atherosclerosis, to medium atherosclerosis, etc. in a training population. In this embodiment, a generalization of the logistic regression model that handles multi-category responses can be used to develop a decision that discriminates between the various trait subgroups found in the training population. For example, measured data for selected molecular markers can be applied to any of the multi-category logit models in order to develop a classifier capable of discriminating between any of a plurality of trait subgroups represented in a training population.

In some embodiments, the analytical process is based on a regression model, preferably a logistic regression model. Such a regression model includes a coefficient for each of the markers in a selected set of markers disclosed herein. In such embodiments, the coefficients for the regression model are computed using, for example, a maximum likelihood approach. In particular embodiments, molecular marker data from the two groups (e.g., healthy and diseased) is used and the dependent variable is the status of the patient corresponding to the marker characteristic data.

Some embodiments of the disclosed methods, assays and kits provide generalizations of the logistic regression model that handle multi-category (polychotomous) responses. Such embodiments can be used to discriminate an organism into one or three or more classifications. Such regression models use multicategory logit models that simultaneously refer to all pairs of categories, and describe the odds of response in one category instead of another. Once the model specifies logits for a certain (J−1) pairs of categories, the rest are redundant.

Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. For use with the disclosed methods, the expression values for the selected set of markers across a subset of the training population serve as the requisite continuous independent variables. The group classification of each of the members of the training population serves as the dichotomous categorical dependent variable.

LDA seeks the linear combination of variables that maximizes the ratio of between-group variance and within-group variance by using the grouping information. Implicitly, the linear weights used by LDA depend on how the expression of a marker across the training set separates in the two groups (e.g., a group that has atherosclerosis and a group that does not have atherosclerosis) and how this expression correlates with the expression of other markers. In some embodiments, LDA is applied to the data matrix of the N members in the training sample by K genes in a combination of genes described in the present disclosure. Then, the linear discriminant of each member of the training population is plotted. Ideally, those members of the training population representing a first subgroup (e.g. those subjects that do not have atherosclerosis) will cluster into one range of linear discriminant values (e.g., negative) and those member of the training population representing a second subgroup (e.g. those subjects that have atherosclerosis) will cluster into a second range of linear discriminant values (e.g., positive). The LDA is considered more successful when the separation between the clusters of discriminant values is larger.

Quadratic discriminant analysis (QDA) takes the same input parameters and returns the same results, as LDA. QDA uses quadratic equations, rather than linear equations, to produce results. LDA and QDA are roughly interchangeable (though there are differences related to the number of subjects required), and which to use is a matter of preference and/or availability of software to support the analysis. Logistic regression takes the same input parameters and returns the same results as LDA and QDA.

One type of analytical process that can be constructed using the expression level of the markers identified herein is a decision tree. Here, the “data analysis algorithm” is any technique that can build the analytical process, whereas the final “decision tree” is the analytical process. An analytical process is constructed using a training population and specific data analysis algorithms. Tree-based methods partition the feature space into a set of rectangles, and then fit a model (like a constant) in each one.

The training population data includes the features (e.g., expression values, or some other observable) for the markers across a training set population. One specific algorithm that can be used to construct an analytical process is a classification and regression tree (CART). Other specific decision tree algorithms include, but are not limited to, ID3, C4.5, MART, and Random Forests. All such algorithms are known in the art.

In some embodiments of the disclosed methods, assays and kits, decision trees are used to classify patients using expression data for a selected set of markers. Decision tree algorithms belong to the class of supervised learning algorithms. The aim of a decision tree is to induce an analytical process (a tree) from real-world example data. This tree can be used to classify unseen examples which have not been used to derive the decision tree.

A decision tree is derived from training data. An example contains values for the different attributes and what class the example belongs. In one embodiment, the training data is expression data for a combination of markers described herein across the training population.

The following algorithm describes a decision tree derivation:
Tree (Examples,Class,Attributes)
Create a root node
If all Examples have the same Class value, give the root this label
Else if Attributes is empty label the root according to the most common value
Else begin
Calculate the information gain for each attribute
Select the attribute A with highest information gain and make this the root attribute
For each possible value, v, of this attribute
Add a new branch below the root, corresponding to A=v Let Examples(v) be those examples with A=v
If Examples(v) is empty, make the new branch a leaf node labeled with the most common value among Examples
Else let the new branch be the tree created by Tree (Examples(v),Class,Attributes-{A})
End.

A more detailed description of the calculation of information gain is shown in the following. If the possible classes vi of the examples have probabilities P(vi) then the information content I of the actual answer is given by:

$$I ( P ( {V}_{1} ) , … , P ( {V}_{n} ) ) = {\sum }_{i = 1}^{n} - P ( {v}_{1} ) {log}_{2} P ( {v}_{i} ) .$$

The I-value shows how much information is needed in order to be able to describe the outcome of a classification for the specific dataset used. Supposing that the dataset contains p positive (e.g. has atherosclerosis) and n negative (e.g. healthy) examples (e.g. individuals), the information contained in a correct answer is:

$$I ( \frac{p}{p + n} \cdot \frac{n}{p + n} ) = - \frac{p}{p + n} {log}_{2} \frac{p}{p + n} - \frac{n}{p + n} {log}_{2} \frac{n}{p + n}$$

where log₂ is the logarithm using base two. By testing single attributes the amount of information needed to make a correct classification can be reduced. The remainder for a specific attribute A (e.g. a marker) shows how much the information that is needed can be reduced.

$$Remainder ( A ) = {\sum }_{i = 1}^{v} \frac{{p}_{i} + {n}_{i}}{p + n} I ( \frac{{p}_{i}}{{p}_{i} + {n}_{i}} \cdot \frac{{n}_{i}}{{p}_{i} + {n}_{i}} )$$

where “v” is the number of unique attribute values for attribute A in a certain dataset, “i” is a certain attribute value, “pᵢ” is the number of examples for attribute A where the classification is positive (e.g. atherosclerotic), “nᵢ” is the number of examples for attribute A where the classification is negative (e.g. healthy).

The information gain of a specific attribute A is calculated as the difference between the information content for the classes and the remainder of attribute A:

$$Gain ( A ) = I ( \frac{p}{p + n} \cdot \frac{n}{p + n} ) - Remainder ( A ) .$$

The information gain is used to evaluate how important the different attributes are for the classification (how well they split up the examples), and the attribute with the highest information.

In general there are a number of different decision tree algorithms, including but not limited to, classification and regression trees (CART), multivariate decision trees, ID3, and C4.5.

In one embodiment when a decision tree is used, the expression data for a selected set of markers across a training population is standardized to have mean zero and unit variance. The members of the training population are randomly divided into a training set and a test set. For example, in one embodiment, two thirds of the members of the training population are placed in the training set and one third of the members of the training population are placed in the test set. The expression values for a select combination of markers described herein is used to construct the analytical process. Then, the ability for the analytical process to correctly classify members in the test set is determined. In some embodiments, this computation is performed several times for a given combination of markers. In each iteration of the computation, the members of the training population are randomly assigned to the training set and the test set. Then, the quality of the combination of molecular markers is taken as the average of each such iteration of the analytical process computation.

In addition to univariate decision trees in which each split is based on an expression level for a corresponding marker, among the set of markers disclosed herein, or the expression level of two such markers, multivariate decision trees can be implemented as an analytical process. In such multivariate decision trees, some or all of the decisions actually comprise a linear combination of expression levels for a plurality of markers. Such a linear combination can be trained using known techniques such as gradient descent on a classification or by the use of a sum-squared-error criterion.

To illustrate such an analytical process, consider the expression: 0.04x₁+0.16x₂<500. Here, x₁ and x₂ refer to two different features for two different markers from among the markers disclosed herein. To poll the analytical process, the values of features x₁ and x₂ are obtained from the measurements obtained from the unclassified subject. These values are then inserted into the equation. If a value of less than 500 is computed, then a first branch in the decision tree is taken. Otherwise, a second branch in the decision tree is taken.

Another approach that can be used in the present disclosure is multivariate adaptive regression splines (MARS). MARS is an adaptive procedure for regression, and is well suited for the high-dimensional problems addressed by the methods disclosed herein. MARS can be viewed as a generalization of stepwise linear regression or a modification of the CART method to improve the performance of CART in the regression setting.

In some embodiments, the expression values for a selected set of markers are used to cluster a training set. For example, consider the case in which ten markers are used. Each member m of the training population will have expression values for each of the ten markers. Such values from a member m in the training population define the vector:

x₁ₘx₂ₘx₃ₘx₄ₘx₅ₘx₆ₘx₇ₘx₈ₘx₉ₘx₁₀ₘ
where Xᵢₘ is the expression level of the i^{th} marker in subject m. If there are m organisms in the training set, selection of i markers will define m vectors. Note that the methods disclosed herein do not require that each the expression value of every single marker used in the vectors be represented in every single vector m. In other words, data from a subject in which one of the i^{th} marker is not found can still be used for clustering. In such instances, the missing expression value is assigned either a “zero” or some other normalized value. In some embodiments, prior to clustering, the expression values are normalized to have a mean value of zero and unit variance.

Those members of the training population that exhibit similar expression patterns across the training group will tend to cluster together. A particular combination of markers is considered to be a good classifier in this aspect of the methods disclosed herein when the vectors cluster into the trait groups found in the training population. For instance, if the training population includes healthy patients and atherosclerotic patients, a clustering classifier will cluster the population into two groups, with each group uniquely representing either healthy patients and atherosclerotic patients.

The clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

One way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.”

Once a method for measuring “similarity” or “dissimilarity” between points in a dataset has been selected, clustering requires a criterion function that measures the clustering quality of any partition of the data. Partitions of the data set that extremize the criterion function are used to cluster the data. Particular exemplary clustering techniques that can be used with the methods disclosed herein include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Principal component analysis (PCA) has been proposed to analyze biomarker data. More generally, PCA can be used to analyze feature value data of markers disclosed herein in order to construct an analytical process that discriminates one class of patients from another (e.g., those who have atherosclerosis and those who do not). Principal component analysis is a classical technique to reduce the dimensionality of a data set by transforming the data to a new set of variable (principal components) that summarize the features of the data.

A few non-limiting examples of PCA are as follows. Principal components (PCs) are uncorrelate and are ordered such that the k^{th} PC has the k^{th} largest variance among PCs. The k^{th} PC can be interpreted as the direction that maximizes the variation of the projections of the data points such that it is orthogonal to the first k−1 PCs. The first few PCs capture most of the variation in the data set. In contrast, the last few PCs are often assumed to capture only the residual “noise” in the data.

PCA can also be used to create an analytical process as disclosed herein. In such an approach, vectors for a selected set of markers can be constructed in the same manner described for clustering. In fact, the set of vectors, where each vector represents the expression values for the select markers from a particular member of the training population, can be considered a matrix. In some embodiments, this matrix is represented in a Free-Wilson method of qualitative binary description of monomers, and distributed in a maximally compressed space using PCA so that the first principal component (PC) captures the largest amount of variance information possible, the second principal component (PC) captures the second largest amount of all variance information, and so forth until all variance information in the matrix has been accounted for.

Then, each of the vectors (where each vector represents a member of the training population) is plotted. Many different types of plots are possible. In some embodiments, a one-dimensional plot is made. In this one-dimensional plot, the value for the first principal component from each of the members of the training population is plotted. In this form of plot, the expectation is that members of a first group (e.g. healthy patients) will cluster in one range of first principal component values and members of a second group (e.g., patients with atherosclerosis) will cluster in a second range of first principal component values (one of skill in the art would appreciate that the distribution of the marker values need to exhibit no elongation in any of the variables for this to be effective).

In one example, the training population comprises two groups: healthy patients and patients with atherosclerosis. The first principal component is computed using the marker expression values for the selected markers across the entire training population data set. Then, each member of the training set is plotted as a function of the value for the first principal component. In this example, those members of the training population in which the first principal component is positive are the healthy patients and those members of the training population in which the first principal component is negative are atherosclerotic patients.

In some embodiments, the members of the training population are plotted against more than one principal component. For example, in some embodiments, the members of the training population are plotted on a two-dimensional plot in which the first dimension is the first principal component and the second dimension is the second principal component. In such a two-dimensional plot, the expectation is that members of each subgroup represented in the training population will cluster into discrete groups. For example, a first cluster of members in the two-dimensional plot will represent subjects with mild atherosclerosis, a second cluster of members in the two-dimensional plot will represent subjects with moderate atherosclerosis, and so forth.

In some embodiments, the members of the training population are plotted against more than two principal components and a determination is made as to whether the members of the training population are clustering into groups that each uniquely represents a subgroup found in the training population. In some embodiments, principal component analysis is performed by using the R mva package (a statistical analysis language), which is known to those of skill in the art.

Nearest neighbor classifiers are memory-based and require no model to be fit. Given a query point x₀, the k training points x₍ᵣ₎, r, . . . , k closest in distance to x₀ are identified and then the point x₀ is classified using the k nearest neighbors. Ties can be broken at random. In some embodiments, Euclidean distance in feature space is used to determine distance as:

d₍ᵣ₎=∥x₍ᵣ₎−xᵣ∥

Typically, when the nearest neighbor algorithm is used, the expression data used to compute the linear discriminant is standardized to have mean zero and variance 1. For the disclosed methods, the members of the training population are randomly divided into a training set and a test set. For example, in one embodiment, two thirds of the members of the training population are placed in the training set and one third of the members of the training population are placed in the test set. Profiles of a selected set of markers disclosed herein represents the feature space into which members of the test set are plotted. Next, the ability of the training set to correctly characterize the members of the test set is computed. In some embodiments, nearest neighbor computation is performed several times for a given combination of markers. In each iteration of the computation, the members of the training population are randomly assigned to the training set and the test set. Then, the quality of the combination of markers is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors.

Inspired by the process of biological evolution, evolutionary methods of classifier design employ a stochastic search for an analytical process. In broad overview, such methods create several analytical processes—a population—from measurements such as the biomarker generated datasets disclosed herein. Each analytical process varies somewhat from the other. Next, the analytical processes are scored on data across the training datasets. In keeping with the analogy with biological evolution, the resulting (scalar) score is sometimes called the fitness. The analytical processes are ranked according to their score and the best analytical processes are retained (some portion of the total population of analytical processes). Again, in keeping with biological terminology, this is called survival of the fittest. The analytical processes are stochastically altered in the next generation—the children or offspring. Some offspring analytical processes will have higher scores than their parent in the previous generation, some will have lower scores. The overall process is then repeated for the subsequent generation: The analytical processes are scored and the best ones are retained, randomly altered to give yet another generation, and so on. In part, because of the ranking, each generation has, on average, a slightly higher score than the previous one. The process is halted when the single best analytical process in a generation has a score that exceeds a desired criterion value.

Bagging, boosting, the random subspace method, and additive trees are data analysis algorithms known as combining techniques that can be used to improve weak analytical processes. These techniques are designed for, and usually applied to, decision trees, such as the decision trees described above. In addition, such techniques can also be useful in analytical processes developed using other types of data analysis algorithms such as linear discriminant analysis.

In bagging, one samples the training datasets, generating random independent bootstrap replicates, constructs the analytical processes on each of these, and aggregates them by a simple majority vote in the final analytical process. In boosting, analytical processes are constructed on weighted versions of the training set, which are dependent on previous analytical process results. Initially, all objects have equal weights, and the first analytical process is constructed on this data set. Then, weights are changed according to the performance of the analytical process. Erroneously classified objects get larger weights, and the next analytical process is boosted on the reweighted training set. In this way, a sequence of training sets and classifiers is obtained, which is then combined by simple majority voting or by weighted majority voting in the final decision.

To illustrate boosting, consider the case where there are two phenotypic groups exhibited by the population under study, phenotype 1 (e.g., poor prognosis patients), and phenotype 2 (e.g., good prognosis patients). Given a vector of molecular markers X, a classifier G(X) produces a prediction taking one of the type values in the two value set: {phenotype 1, phenotype 2}. The error rate on the training sample is

$$err = 1 / N {\sum }_{i = 1}^{N} I ( {y}_{i} \neq G ( {x}_{i} ) ) ,$$

where N is the number of subjects in the training set (the sum total of the subjects that have either phenotype 1 or phenotype 2). For example, if there are 35 healthy patients and 46 sclerotic patients, N is 81.

A weak analytical process is one Whose error rate is only slightly better than random guessing. In the boosting algorithm, the weak analytical process is repeatedly applied to modified versions of the data, thereby producing a sequence of weak classifiers Gₘ(x), m=1, 2, . . . , M. The predictions from all of the classifiers in this sequence are then combined through a weighted majority vote to produce the final prediction:

$$G ( x ) = sign ( {\sum }_{m = 1}^{M} {a}_{m} {G}_{m} ( x ) )$$

1. Initialize the observation weights wᵢ=1/N, 1=1, 2, . . . , N
2. For m=1 to M:

(a) Fit an analytical process Gₘ(x) to the training set using weights wᵢ.

(b) Compute

$$err = \frac{{\sum }_{i = 1}^{N} {w}_{i} I ( {y}_{i} \neq {G}_{m} ( {x}_{i} ) )}{{\sum }_{i = 1}^{N} {w}_{i}}$$

(c) Compute aₘ=log((1−errₘ/errₘ).

(d) Set wᵢ [symbol] wᵢ exp[αₘI(yᵢ≠Gₘ(xᵢ))], i=1, 2, . . . , N.

### 3. Output

Here α₁, α₂, . . . , αₘ are computed by the boosting algorithm and their purpose is to weigh the contribution of each respective Gₘ(x). Their effect is to give higher influence to the more accurate classifiers in the sequence.

The data modifications at each boosting step consist of applying weights w₁, w₂, . . . , wₙ to each of the training observations (xᵢ, yᵢ), i=1, 2, . . . , N. Initially all the weights are set to wᵢ=1/N, so that the first step simply trains the analytical process on the data in the usual manner. For each successive iteration m=2, 3, . . . , M the observation weights are individually modified and the analytical process is reapplied to the weighted observations. At stem m, those observations that were misclassified by the analytical process Gₘ₋₁(x) induced at the previous step have their weights increased, whereas the weights are decreased for those that were classified correctly. Thus as iterations proceed, observations that are difficult to correctly classify receive ever-increasing influence. Each successive analytical process is thereby forced to concentrate on those training observations that are missed by previous ones in the sequence.

The exemplary boosting algorithm is summarized as follows:

1. Initialize the observation weights wᵢ=1/N, i=1, 2, . . . , N.
2. For m=1 to M:

(a) Fit an analytical process Gₘ(x) to the training set using weights wᵢ,

(b) Compute

$$err = \frac{{\sum }_{i = 1}^{N} {w}_{i} I ( {y}_{i} \neq {G}_{m} ( {x}_{i} ) )}{{\sum }_{i = 1}^{N} {w}_{i}}$$

(C) Compute αₘ=log((1−errₘ)/errₘ).

(d) Set wᵢ←→wᵢ exp[αₘI(yᵢ≠Gₘ(xᵢ))], i=1, 2, . . . , N.

### 3. Output

$$G ( x ) = sign \vert {\sum }_{m = 1}^{M} {a}_{m} {G}_{m} ( x ) \vert$$

In the algorithm m, the current classifier Gₘ(x) is induced on the weighted observations at line 2a. The resulting weighted error rate is computed at line 2b. Line 2c calculates the weight αₘ given to Gₘ(x) in producing the final classifier Gₘ (line 3). The individual weights of each of the observations are updated for the next iteration at line 2d. Observations misclassified by Gₘ(x) have their weights scaled by a factor exp(αₘ), increasing their relative influence for inducing the next classifier Gₘ+I(x) in the sequence. In some embodiments, boosting or adaptive boosting methods are used.

In some embodiments, feature preselection is performed using a technique such as the nonparametric scoring method. Feature preselection is a form of dimensionality reduction in which the markers that discriminate between classifications the best are selected for use in the classifier. Then, the LogitBoost procedure is used rather than the boosting procedure. In some embodiments, the boosting and other classification methods are used in the disclosed methods.

In the random subspace method, classifiers are constructed in random subspaces of the data feature space. These classifiers are usually combined by simple majority voting in the final decision rule (i.e., analytical process).

As indicated, the statistical techniques described herein are merely examples of the types of algorithms and models that can be used to identify a preferred group of markers to include in a dataset and to generate an analytical process that can be used to generate a result using the dataset. Further, combinations of the techniques described above and elsewhere can be used either for the same task or each for a different task. Some combinations, such as the use of the combination of decision trees and boosting, have been described. However, many other combinations are possible. By way of example, other statistical techniques in the art such as Projection Pursuit and Weighted Voting can be used to identify a preferred group of markers to include in a dataset and to generate an analytical process that can be used to generate a result using the dataset.

An optimum number of dataset components to be evaluated in an analytical process can be determined. When using the learning algorithms described above to develop a predictive model, one of skill in the art may select a subset of markers, i.e. at least 3, at least 4, at least 5, at least 6, up to the complete set of markers, to define the analytical process. Usually a subset of markers will be chosen that provides for the needs of the quantitative sample analysis, e.g. availability of reagents, convenience of quantitation, etc., while maintaining a highly accurate predictive model.

The selection of a number of informative markers for building classification models requires the definition of a performance metric and a user-defined threshold for producing a model with useful predictive ability based on this metric. For example, the performance metric may be the AUC, the sensitivity and/or specificity of the prediction as well as the overall accuracy of the prediction model.

The predictive ability of a model may be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold may refer to a predictive model that will classify a sample with an AUC of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity may be at least about at least about 0.7, at least about 0.75, at least about 0.8, at least about 0.85, at least about 0.9, or higher.

Various methods are used in a training model. The selection of a subset of markers may be via a forward selection or a backward selection of a marker subset. The number of markers to be selected is that which will optimize the performance of a model without the use of all the markers. One way to define the optimum number of terms is to choose the number of terms that produce a model with desired predictive ability (e.g. an AUC>0.75, or equivalent measures of sensitivity/specificity) that lies no more than one standard error from the maximum value obtained for this metric using any combination and number of terms used for the given algorithm.

As described above, quantitative data for components of the dataset are inputted into an analytic process and used to generate a result. The result can be any type of information useful for making an atherosclerotic classification, e.g. a classification, a continuous variable, or a vector. For example, the value of a continuous variable or vector may be used to determine the likelihood that a sample is associated with a particular classification.

Atherosclerotic classification refer to any type of information or the generation of any type of information associated with an atherosclerotic condition, for example, diagnosis, staging, assessing extent of atherosclerotic progression, prognosis, monitoring, therapeutic response to treatments, screening to identify compounds that act via similar mechanisms as known atherosclerotic treatments, prediction of pseudo-coronary calcium score, stable (i.e., angina) vs. unstable (i.e., myocardial infarction), identifying complications of atherosclerotic disease, etc.

In a preferred embodiment, the result is used for diagnosis or detection of the occurrence of an atherosclerosis, particularly where such atherosclerosis is indicative of a propensity for myocardial infarction, heart failure, etc. In this embodiment, a reference or training set containing “healthy” and “atherosclerotic” samples is used to develop a predictive model. A dataset, preferably containing protein expression levels of markers indicative of the atherosclerosis, is then inputted into the predictive model in order to generate a result. The result may classify the sample as either “healthy” or “atherosclerotic”. In other embodiments, the result is a continuous variable providing information useful for classifying the sample, e.g., where a high value indicates a high probability of being an “atherosclerotic” sample and a low value indicates a low probability of being a “healthy” sample.

In other embodiments, the result is used for atherosclerosis staging. In this embodiment, a reference or training dataset containing samples from individuals with disease at different stages is used to develop a predictive model. The model may be a simple comparison of an individual dataset against one or more datasets obtained from disease samples of known stage or a more complex multivariate classification model. In certain embodiments, inputting a dataset into the model will generate a result classifying the sample from which the dataset is generated as being at a specified cardiovascular disease stage. Similar methods may be used to provide atherosclerosis prognosis, except that the reference or training set will include data obtained from individuals who develop disease and those who fail to develop disease at a later time.

In other embodiments, the result is used to determine response to atherosclerotic disease treatments. In this embodiment, the reference or training dataset and the predictive model is the same as that used to diagnose atherosclerosis (samples of from individuals with disease and those without). However, instead of inputting a dataset composed of samples from individuals with an unknown diagnosis, the dataset is composed of individuals with known disease which have been administered a particular treatment and it is determined whether the samples trend toward or lie within a normal, healthy classification versus an atherosclerotic disease classification.

Treatment as used herein can include, without limitation, a follow-up checkup in 3, 6, or 12 months; pharmacologic intervention such as beta-blocker, calcium channel blocker, aspirin, cholesterol lowering agents, etc; and/or further testing to determine the existence or degree of cardiovascular condition/disease. In certain instances, no immediate treatment will be required.

In another embodiment, the result is used for drug screening, i.e., identifying compounds that act via similar mechanisms as known atherosclerotic drug treatments. In this embodiment, a reference or training set containing individuals treated with a known atherosclerotic drug treatment and those not treated with the particular treatment can be used develop a predictive model. A dataset from individuals treated with a compound with an unknown mechanism is input into the model. If the result indicates that the sample can be classified as coming from a subject dosed with a known atherosclerotic drug treatment, then the new compound is likely to act via the same mechanism.

In preferred embodiments, the result is used to determine a “pseudo-coronary calcium score,” which is a quantitative measure that correlates to coronary calcium score (CCS). CCS is a clinical cardiovascular disease screening technique which measures overall atherosclerotic plaque burden. Various different types of imaging techniques can be used to quantitate the calcium area and density of atherosclerotic plaques. When electron-beam CT and multidetector CT are used, CCS is a function of the x-ray attenuation coefficient and the area of calcium deposits. Typically, a score of 0 is considered to indicate no atherosclerotic plaque burden, >0 to 10 to indicate minimal evidence of plaque burden, 11 to 100 to indicate at least mild evidence of plaque burden, 101 to 400 to indicate at least moderate evidence of plaque burden, and over 400 as being extensive evidence of plaque burden. CCS used in conjunction with traditional risk factors improves predictive ability for complications of cardiovascular disease. In addition, the CCS is also capable of acting as an independent predictor of cardiovascular disease complications.

A reference or training set containing individuals with high and low coronary calcium scores can be used to develop a model for predicting the pseudo-coronary calcium score of an individual. This predicted pseudo-coronary calcium score is useful for diagnosing and monitoring atherosclerosis. In some embodiments, the pseudo-coronary calcium score is used in conjunction with other known cardiovascular diagnosis and monitoring methods, such as actual coronary calcium score derived from imaging techniques to diagnose and monitor cardiovascular disease.

One of skill will also recognize that the results generated using these methods can be used in conjunction with any number of the various other methods known to those of skill in the art for diagnosing and monitoring cardiovascular disease.

Also provided are reagents and kits thereof for practicing one or more of the above-described methods. The subject reagents and kits thereof may vary greatly. Reagents of interest include reagents specifically designed for use in production of the above described expression profiles of circulating miRNA markers, protein biomarkers, or a combination of miRNA and protein markers associated with atherosclerotic conditions.

In one embodiment a kit for assessing the cardiovascular health of a human to determine the need for or effectiveness of a treatment regimen is provided, which comprises: an assay for determining levels of at least two miRNA markers selected from the miRNAs in Table 20 in the biological sample; instructions for obtaining a dataset comprised of the levels of each miRNA marker, inputting the data into an analytical classification process that uses the data to classify the biological sample, wherein the classification is selected from the group consisting of an atherosclerotic cardiovascular disease classification, a healthy classification, a medication exposure classification, a no medication exposure classification; and classifying the biological sample according to the output of the classification process and determining a treatment regimen for the human based on the classification.

In certain embodiments, the kit further comprises an assay for determining levels of at least three protein biomarker selected from the group consisting IL-16, sFas, Fas ligand, MCP-3, HGF, CTACK, EOTAXIN, adiponectin, IL-18, TIMP.4, TIMP.1, CRP, VEGF, and EGF in the biological sample; and instructions for obtaining a dataset comprised of the individual levels of the protein markers, inputting the data of the miRNA and protein markers into an analytical classification process that uses the data to classify the biological sample, wherein the classification is selected from the group consisting of an atherosclerotic cardiovascular disease classification, a healthy classification, a medication exposure classification, a no medication exposure classification; and classifying the biological sample according to the output of the classification process and determining a treatment regimen for the human based on the classification.

One type of such reagent is an array or kit of antibodies that bind to a marker set of interest. A variety of different array formats are known in the art, with a wide variety of different probe structures, substrate compositions and attachment technologies. Representative array or kit compositions of interest include or consist of reagents for quantitation of at least 2, at least 3, at least 4, at least 5 or more miRNA markers alone or in combination with protein markers. In this regard, the reagent can be for quantitation of at least 1, at least 2, at least 3, at least 4, at least 5 miRNA markers selected from the miRNAs listed in Table 1 and preferably, the miRNAs listed in Table 20.

TABLE 1

| Coverage Human | | SEQ ID | Target sequence |
|---|---|---|---|
| microRNA | Target sequence | No: | accession |
| hsa-miR-155* | CUCCUACAUAUUAGCAUUAACA | 1 | MIMAT0004658 |
| hsa-miR-486-5p | UCCUGUACUGAGCUGCCCCGAG | 2 | MIMAT0002177 |
| hsa-miR-596 | AAGCCUGCCCGGCUCCUCGGG | 3 | MIMAT0003264 |
| hsa-miR-532-3p | CCUCCCACACCCAAGGCUUGCA | 4 | MIMAT0004780 |
| hsa-miR-1238 | CUUCCUCGUCUGUCUGCCCC | 5 | MIMAT0005593 |
| hsa-miR-34b | CAAUCACUAACUCCACUGCCAU | 6 | MIMAT0004676 |
| hsa-miR-151-5p | UCGAGGAGCUCACAGUCUAGU | 7 | MIMAT0004697 |
| hsa-miR-361-3p | UCCCCCAGGUGUGAUUCUGAUUU | 8 | MIMAT0004682 |
| hsa-miR-211 | UUCCCUUUGUCAUCCUUCGCCU | 9 | MIMAT0000268 |
| hsa-miR-217 | UACUGCAUCAGGAACUGAUUGGA | 10 | MIMAT0000274 |
| hsa-miR-370 | GCCUGCUGGGGUGGAACCUGGU | 11 | MIMAT0000722 |
| hsa-miR-483-3p | UCACUCCUCUCCUCCCGUCUU | 12 | MIMAT0002173 |
| hsa-miR-520e | AAAGUGCUUCCUUUUUGAGGG | 13 | MIMAT0002825 |
| hsa-miR-409-5p | AGGUUACCCGAGCAACUUUGCAU | 14 | MIMAT0001638 |
| hsa-miR-186 | CAAAGAAUUCUCCUUUUGGGCU | 15 | MIMAT0000456 |
| hsa-miR-519c-3p | AAAGUGCAUCUUUUUAGAGGAU | 16 | MIMAT0002832 |
| hsa-miR-330-3p | GCAAAGCACACGGCCUGCAGAGA | 17 | MIMAT0000751 |
| hsa-miR-187 | UCGUGUCUUGUGUUGCAGCCGG | 18 | MIMAT0000262 |
| hsa-miR-623 | AUCCCUUGCAGGGGCUGUUGGGU | 19 | MIMAT0003292 |
| hsa-miR-106b* | CCGCACUGUGGGUACUUGCUGC | 20 | MIMAT0004672 |
| hsa-miR-583 | CAAAGAGGAAGGUCCCAUUAC | 21 | MIMAT0003248 |
| hsa-miR-135a* | UAUAGGGAUUGGAGCCGUGGCG | 22 | MIMAT0004595 |
| hsa-miR-30d* | CUUUCAGUCAGAUGUUUGCUGC | 23 | MIMAT0004551 |
| hsa-miR-671-3p | UCCGGUUCUCAGGGCUCCACC | 24 | MIMAT0004819 |
| hsa-miR-1270 | CUGGAGAUAUGGAAGAGCUGUGU | 25 | MIMAT0005924 |
| hsa-miR-129-3p | AAGCCCUUACCCCAAAAAGCAU | 26 | MIMAT0004605 |
| hsa-miR-647 | GUGGCUGCACUCACUUCCUUC | 27 | MIMAT0003317 |
| hsa-miR-934 | UGUCUACUACUGGAGACACUGG | 28 | MIMAT0004977 |
| hsa-miR-519e* | UUCUCCAAAAGGGAGCACUUUC | 29 | MIMAT0002828 |
| hsa-miR-524-3p | GAAGGCGCUUCCCUUUGGAGU | 30 | MIMAT0002850 |
| hsa-miR-25* | AGGCGGAGACUUGGGCAAUUG | 31 | MIMAT0004498 |
| hsa-miR-221* | ACCUGGCAUACAAUGUAGAUUU | 32 | MIMAT0004568 |
| hsa-miR-302d* | ACUUUAACAUGGAGGCACUUGC | 33 | MIMAT0004685 |
| hsa-miR-455-3p | GCAGUCCAUGGGCAUAUACAC | 34 | MIMAT0004784 |
| hsa-miR-433 | AUCAUGAUGGGCUCCUCGGUGU | 35 | MIMAT0001627 |
| hsa-miR-139-5p | UCUACAGUGCACGUGUCUCCAG | 36 | MIMAT0000250 |
| hsa-miR-425* | AUCGGGAAUGUCGUGUCCGCCC | 37 | MIMAT0001343 |
| hsa-miR-30a | UGUAAACAUCCUCGACUGGAAG | 38 | MIMAT0000087 |
| hsa-miR-520d-3p | AAAGUGCUUCUCUUUGGUGGGU | 39 | MIMAT0002856 |
| hsa-miR-611 | GCGAGGACCCCUCGGGGUCUGAC | 40 | MIMAT0003279 |
| hsa-miR-410 | AAUAUAACACAGAUGGCCUGU | 41 | MIMAT0002171 |
| hsa-miR-502-3p | AAUGCACCUGGGCAAGGAUUCA | 42 | MIMAT0004775 |
| hsa-miR-1200 | CUCCUGAGCCAUUCUGAGCCUC | 43 | MIMAT0005863 |
| hsa-miR-1224-3p | CCCCACCUCCUCUCUCCUCAG | 44 | MIMAT0005459 |
| hsa-miR-511 | GUGUCUUUUGCUCUGCAGUCA | 45 | MIMAT0002808 |
| hsa-miR-148b | UCAGUGCAUCACAGAACUUUGU | 46 | MIMAT0000759 |
| hsa-miR-127-3p | UCGGAUCCGUCUGAGCUUGGCU | 47 | MIMAT0000446 |
| hsa-miR-485-3p | GUCAUACACGGCUCUCCUCUCU | 48 | MIMAT0002176 |
| hsa-miR-1181 | CCGUCGCCGCCACCCGAGCCG | 49 | MIMAT0005826 |
| hsa-miR-518e | AAAGCGCUUCCCUUCAGAGUG | 50 | MIMAT0002861 |
| hsa-miR-20a* | ACUGCAUUAUGAGCACUUAAAG | 51 | MIMAT0004493 |
| hsa-miR-492 | AGGACCUGCGGGACAAGAUUCUU | 52 | MIMAT0002812 |
| hsa-miR-654-3p | UAUGUCUGCUGACCAUCACCUU | 53 | MIMAT0004814 |
| hsa-miR-520g | ACAAAGUGCUUCCCUUUAGAGUGU | 54 | MIMAT0002858 |
| hsa-miR-1264 | CAAGUCUUAUUUGAGCACCUGUU | 55 | MIMAT0005791 |
| hsa-miR-324-5p | CGCAUCCCCUAGGGCAUUGGUGU | 56 | MIMAT0000761 |
| hsa-miR-129* | AAGCCCUUACCCCAAAAAGUAU | 57 | MIMAT0004548 |
| hsa-miR-1256 | AGGCAUUGACUUCUCACUAGCU | 58 | MIMAT0005907 |
| hsa-miR-937 | AUCCGCGCUCUGACUCUCUGCC | 59 | MIMAT0004980 |
| hsa-miR-369-5p | AGAUCGACCGUGUUAUAUUCGC | 60 | MIMAT0001621 |
| hsa-miR-519d | CAAAGUGCCUCCCUUUAGAGUG | 61 | MIMAT0002853 |
| hsa-miR-103 | AGCAGCAUUGUACAGGGCUAUGA | 62 | MIMAT0000101 |
| hsa-miR-99b* | CAAGCUCGUGUCUGUGGGUCCG | 63 | MIMAT0004678 |
| hsa-miR-193b* | CGGGGUUUUGAGGGCGAGAUGA | 64 | MIMAT0004767 |
| hsa-miR-15a | UAGCAGCACAUAAUGGUUUGUG | 65 | MIMAT0000068 |
| hsa-miR-551b | GCGACCCAUACUUGGUUUCAG | 66 | MIMAT0003233 |
| hsa-miR-612 | GCUGGGCAGGGCUUCUGAGCUCC | 67 | MIMAT0003280 |
| | UU | | |
| hsa-miR-1237 | UCCUUCUGCUCCGUCCCCCAG | 68 | MIMAT0005592 |
| hsa-miR-595 | GAAGUGUGCCGUGGUGUGUCU | 69 | MIMAT0003263 |
| hsa-miR-765 | UGGAGGAGAAGGAAGGUGAUG | 70 | MIMAT0003945 |
| hsa-miR-582-3p | UAACUGGUUGAACAACUGAACC | 71 | MIMAT0004797 |
| hsa-let-7b | UGAGGUAGUAGGUUGUGUGGUU | 72 | MIMAT0000063 |
| hsa-miR-520a-3p | AAAGUGCUUCCCUUUGGACUGU | 73 | MIMAT0002834 |
| hsa-miR-604 | AGGCUGCGGAAUUCAGGAC | 74 | MIMAT0003272 |
| hsa-miR-600 | ACUUACAGACAAGAGCCUUGCUC | 75 | MIMAT0003268 |
| hsa-miR-508-5p | UACUCCAGAGGGCGUCACUCAUG | 76 | MIMAT0004778 |
| hsa-miR-27a | UUCACAGUGGCUAAGUUCCGC | 77 | MIMAT0000084 |
| hsa-miR-31* | UGCUAUGCCAACAUAUUGCCAU | 78 | MIMAT0004504 |
| hsa-miR-194 | UGUAACAGCAACUCCAUGUGGA | 79 | MIMAT0000460 |
| hsa-miR-490-5p | CCAUGGAUCUCCAGGUGGGU | 80 | MIMAT0004764 |
| hsa-miR-1265 | CAGGAUGUGGUCAAGUGUUGUU | 81 | MIMAT0005918 |
| hsa-miR-593 | UGUCUCUGCUGGGGUUUCU | 82 | MIMAT0004802 |
| hsa-miR-18b | UAAGGUGCAUCUAGUGCAGUUAG | 83 | MIMAT0001412 |
| hsa-miR-323-5p | AGGUGGUCCGUGGCGCGUUCGC | 84 | MIMAT0004696 |
| hsa-miR-33a* | CAAUGUUUCCACAGUGCAUCAC | 85 | MIMAT0004506 |
| hsa-miR-185* | AGGGGCUGGCUUUCCUCUGGUC | 86 | MIMAT0004611 |
| hsa-miR-720 | UCUCGCUGGGGCCUCCA | 87 | MIMAT0005954 |
| hsa-miR-18b* | UGCCCUAAAUGCCCCUUCUGGC | 88 | MIMAT0004751 |
| hsa-miR-122 | UGGAGUGUGACAAUGGUGUUUG | 89 | MIMAT0000421 |
| hsa-miR-1178 | UUGCUCACUGUUCUUCCCUAG | 90 | MIMAT0005823 |
| hsa-miR-892a | CACUGUGUCCUUUCUGCGUAG | 91 | MIMAT0004907 |
| hsa-miR-149* | AGGGAGGGACGGGGGCUGUGC | 92 | MIMAT0004609 |
| hsa-miR-940 | AAGGCAGGGCCCCCGCUCCCC | 93 | MIMAT0004983 |
| hsa-let-7f-2* | CUAUACAGUCUACUGUCUUUCC | 94 | MIMAT0004487 |
| hsa-miR-154* | AAUCAUACACGGUUGACCUAUU | 95 | MIMAT0000453 |
| hsa-miR-637 | ACUGGGGGCUUUCGGGCUCUGCG | 96 | MIMAT0003307 |
| | U | | |
| hsa-miR-182* | UGGUUCUAGACUUGCCAACUA | 97 | MIMAT0000260 |
| hsa-miR-192, | CUGACCUAUGAAUUGACAGCC | 98 | MIMAT0000222 |
| hsa-miR-519a*, | CUCUAGAGGGAAGCGCUUUCUG | 99 | MIMAT0005452 |
| hsa-miR-518e*, | | | |
| hsa-miR-519b-5p, | | | |
| hsa-miR-519c-5p, | | | |
| hsa-miR-522* & | | | |
| hsa-miR-523* | | | |
| hsa-miR-202 | AGAGGUAUAGGGCAUGGGAA | 100 | MIMAT0002811 |
| hsa-miR-499-5p | UUAAGACUUGCAGUGAUGUUU | 101 | MIMAT0002870 |
| hsa-miR-548i | AAAAGUAAUUGCGGAUUUUGCC | 102 | MIMAT0005935 |
| hsa-miR-769-3p | CUGGGAUCUCCGGGGUCUUGGUU | 103 | MIMAT0003887 |
| hsa-miR-337-3p | CUCCUAUAUGAUGCCUUUCUUC | 104 | MIMAT0000754 |
| hsa-miR-522 | AAAAUGGUUCCCUUUAGAGUGU | 105 | MIMAT0002868 |
| hsa-miR-486-3p | CGGGGCAGCUCAGUACAGGAU | 106 | MIMAT0004762 |
| hsa-miR-17 | CAAAGUGCUUACAGUGCAGGUAG | 107 | MIMAT0000070 |
| hsa-miR-891b | UGCAACUUACCUGAGUCAUUGA | 108 | MIMAT0004913 |
| hsa-miR-181a* | ACCAUCGACCGUUGAUUGUACC | 109 | MIMAT0000270 |
| hsa-miR-525-3p | GAAGGCGCUUCCCUUUAGAGCG | 110 | MIMAT0002839 |
| hsa-miR-603 | CACACACUGCAAUUACUUUUGC | 111 | MIMAT0003271 |
| hsa-miR-889 | UUAAUAUCGGACAACCAUUGU | 112 | MIMAT0004921 |
| hsa-miR-338-5p | AACAAUAUCCUGGUGCUGAGUG | 113 | MIMAT0004701 |
| hsa-miR-298 | AGCAGAAGCAGGGAGGUUCUCCCA | 114 | MIMAT0004901 |
| hsa-miR-616 | AGUCAUUGGAGGGUUUGAGCAG | 115 | MIMAT0004805 |
| hsa-miR-26b* | CCUGUUCUCCAUUACUUGGCUC | 116 | MIMAT0004500 |
| hsa-miR-541* | AAAGGAUUCUGCUGUCGGUCCCAC | 117 | MIMAT0004919 |
| | U | | |
| hsa-miR-28-3p | CACUAGAUUGUGAGCUCCUGGA | 118 | MIMAT0004502 |
| hsa-miR-619 | GACCUGGACAUGUUUGUGCCCAGU | 119 | MIMAT0003288 |
| hsa-miR-148a | UCAGUGCACUACAGAACUUUGU | 120 | MIMAT0000243 |
| hsa-miR-1249 | ACGCCCUUCCCCCCCUUCUUCA | 121 | MIMAT0005901 |
| hsa-miR-1204 | UCGUGGCCUGGUCUCCAUUAU | 122 | MIMAT0005868 |
| hsa-let-7d | AGAGGUAGUAGGUUGCAUAGUU | 123 | MIMAT0000065 |
| hsa-miR-429 | UAAUACUGUCUGGUAAAACCGU | 124 | MIMAT0001536 |
| hsa-miR-453 | AGGUUGUCCGUGGUGAGUUCGCA | 125 | MIMAT0001630 |
| hsa-miR-195* | CCAAUAUUGGCUGUGCUGCUCC | 126 | MIMAT0004615 |
| hsa-miR-132 | UAACAGUCUACAGCCAUGGUCG | 127 | MIMAT0000426 |
| hsa-miR-135b | UAUGGCUUUUCAUUCCUAUGUGA | 128 | MIMAT0000758 |
| hsa-miR-32 | UAUUGCACAUUACUAAGUUGCA | 129 | MIMAT0000090 |
| hsa-miR-29c* | UGACCGAUUUCUCCUGGUGUUC | 130 | MIMAT0004673 |
| hsa-miR-100 | AACCCGUAGAUCCGAACUUGUG | 131 | MIMAT0000098 |
| hsa-miR-512-5p | CACUCAGCCUUGAGGGCACUUUC | 132 | MIMAT0002822 |
| hsa-miR-524-5p | CUACAAAGGGAAGCACUUUCUC | 133 | MIMAT0002849 |
| hsa-miR-885-3p | AGGCAGCGGGGUGUAGUGGAUA | 134 | MIMAT0004948 |
| hsa-miR-372 | AAAGUGCUGCGACAUUUGAGCGU | 135 | MIMAT0000724 |
| hsa-miR-518a-5p, | CUGCAAAGGGAAGCCCUUUC | 136 | MIMAT0005457 |
| hsa-miR-527, | | | |
| hsa-miR-1185 | AGAGGAUACCCUUUGUAUGUU | 137 | MIMAT0005798 |
| hsa-miR-518f | GAAAGCGCUUCUCUUUAGAGG | 138 | MIMAT0002842 |
| hsa-miR-627 | GUGAGUCUCUAAGAAAAGAGGA | 139 | MIMAT0003296 |
| hsa-miR-181a-2* | ACCACUGACCGUUGACUGUACC | 140 | MIMAT0004558 |
| hsa-miR-1205 | UCUGCAGGGUUUGCUUUGAG | 141 | MIMAT0005869 |
| hsa-miR-200b* | CAUCUUACUGGGCAGCAUUGGA | 142 | MIMAT0004571 |
| hsa-miR-645 | UCUAGGCUGGUACUGCUGA | 143 | MIMAT0003315 |
| hsa-miR-649 | AAACCUGUGUUGUUCAAGAGUC | 144 | MIMAT0003319 |
| hsa-miR-1206 | UGUUCAUGUAGAUGUUUAAGC | 145 | MIMAT0005870 |
| hsa-miR-1255b | CGGAUGAGCAAAGAAAGUGGUU | 146 | MIMAT0005945 |
| hsa-miR-329 | AACACACCUGGUUAACCUCUUU | 147 | MIMAT0001629 |
| hsa-miR-498 | UUUCAAGCCAGGGGGCGUUUUUC | 148 | MIMAT0002824 |
| hsa-miR-335 | UCAAGAGCAAUAACGAAAAAUGU | 149 | MIMAT0000765 |
| hsa-miR-199b-5p | CCCAGUGUUUAGACUAUCUGUUC | 150 | MIMAT0000263 |
| hsa-miR-339-5p | UCCCUGUCCUCCAGGAGCUCACG | 151 | MIMAT0000764 |
| hsa-miR-320a | AAAAGCUGGGUUGAGAGGGCGA | 152 | MIMAT0000510 |
| hsa-miR-181d | AACAUUCAUUGUUGUCGGUGGGU | 153 | MIMAT0002821 |
| hsa-miR-331-3p | GCCCCUGGGCCUAUCCUAGAA | 154 | MIMAT0000760 |
| hsa-miR-302a | UAAGUGCUUCCAUGUUUUGGUGA | 155 | MIMAT0000684 |
| hsa-miR-548k | AAAAGUACUUGCGGAUUUUGCU | 156 | MIMAT0005882 |
| hsa-miR-924 | AGAGUCUUGUGAUGUCUUGC | 157 | MIMAT0004974 |
| hsa-miR-339-3p | UGAGCGCCUCGACGACAGAGCCG | 158 | MIMAT0004702 |
| hsa-miR-127-5p | CUGAAGCUCAGAGGGCUCUGAU | 159 | MIMAT0004604 |
| hsa-miR-133b | UUUGGUCCCCUUCAACCAGCUA | 160 | MIMAT0000770 |
| hsa-miR-220a | CCACACCGUAUCUGACACUUU | 161 | MIMAT0000277 |
| hsa-miR-422a | ACUGGACUUAGGGUCAGAAGGC | 162 | MIMAT0001339 |
| hsa-miR-567 | AGUAUGUUCUUCCAGGACAGAAC | 163 | MIMAT0003231 |
| hsa-miR-493* | UUGUACAUGGUAGGCUUUCAUU | 164 | MIMAT0002813 |
| hsa-miR-216a | UAAUCUCAGCUGGCAACUGUGA | 165 | MIMAT0000273 |
| hsa-miR-589 | UGAGAACCACGUCUGCUCUGAG | 166 | MIMAT0004799 |
| hsa-miR-382 | GAAGUUGUUCGUGGUGGAUUCG | 167 | MIMAT0000737 |
| hsa-miR-212 | UAACAGUCUCCAGUCACGGCC | 168 | MIMAT0000269 |
| hsa-miR-26b | UUCAAGUAAUUCAGGAUAGGU | 169 | MIMAT0000083 |
| hsa-miR-363* | CGGGUGGAUCACGAUGCAAUUU | 170 | MIMAT0003385 |
| hsa-miR-1263 | AUGGUACCCUGGCAUACUGAGU | 171 | MIMAT0005915 |
| hsa-miR-873 | GCAGGAACUUGUGAGUCUCCU | 172 | MIMAT0004953 |
| hsa-miR-1183 | CACUGUAGGUGAUGGUGAGAGUG | 173 | MIMAT0005828 |
| | GGCA | | |
| hsa-miR-517c | AUCGUGCAUCCUUUUAGAGUGU | 174 | MIMAT0002866 |
| hsa-miR-501-3p | AAUGCACCCGGGCAAGGAUUCU | 175 | MIMAT0004774 |
| hsa-miR-378 | ACUGGACUUGGAGUCAGAAGG | 176 | MIMAT0000732 |
| hsa-miR-662 | UCCCACGUUGUGGCCCAGCAG | 177 | MIMAT0003325 |
| hsa-miR-552 | AACAGGUGACUGGUUAGACAA | 178 | MIMAT0003215 |
| hsa-miR-134 | UGUGACUGGUUGACCAGAGGGG | 179 | MIMAT0000447 |
| hsa-miR-591 | AGACCAUGGGUUCUCAUUGU | 180 | MIMAT0003259 |
| hsa-miR-26a-1* | CCUAUUCUUGGUUACUUGCACG | 181 | MIMAT0004499 |
| hsa-miR-936 | ACAGUAGAGGGAGGAAUCGCAG | 182 | MIMAT0004979 |
| hsa-miR-195 | UAGCAGCACAGAAAUAUUGGC | 183 | MIMAT0000461 |
| hsa-miR-24-2* | UGCCUACUGAGCUGAAACACAG | 184 | MIMAT0004497 |
| hsa-miR-148a* | AAAGUUCUGAGACACUCCGACU | 185 | MIMAT0004549 |
| hsa-miR-450b-5p | UUUUGCAAUAUGUUCCUGAAUA | 186 | MIMAT0004909 |
| hsa-miR-143 | UGAGAUGAAGCACUGUAGCUC | 187 | MIMAT0000435 |
| hsa-miR-145* | GGAUUCCUGGAAAUACUGUUCU | 188 | MIMAT0004601 |
| hsa-miR-105* | ACGGAUGUUUGAGCAUGUGCUA | 189 | MIMAT0004516 |
| hsa-miR-302c* | UUUAACAUGGGGGUACCUGCUG | 190 | MIMAT0000716 |
| hsa-miR-576-3p | AAGAUGUGGAAAAAUUGGAAUC | 191 | MIMAT0004796 |
| hsa-miR-191* | GCUGCGCUUGGAUUUCGUCCCC | 192 | MIMAT0001618 |
| hsa-miR-770-5p | UCCAGUACCACGUGUCAGGGCCA | 193 | MIMAT0003948 |
| hsa-miR-542-5p | UCGGGGAUCAUCAUGUCACGAGA | 194 | MIMAT0003340 |
| hsa-miR-659 | CUUGGUUCAGGGAGGGUCCCCA | 195 | MIMAT0003337 |
| hsa-miR-1227 | CGUGCCACCCUUUUCCCCAG | 196 | MIMAT0005580 |
| hsa-miR-452* | CUCAUCUGCAAAGAAGUAAGUG | 197 | MIMAT0001636 |
| hsa-miR-491-3p | CUUAUGCAAGAUUCCCUUCUAC | 198 | MIMAT0004765 |
| hsa-miR-380* | UGGUUGACCAUAGAACAUGCGC | 199 | MIMAT0000734 |
| hsa-miR-194* | CCAGUGGGGCUGCUGUUAUCUG | 200 | MIMAT0004671 |
| hsa-miR-586 | UAUGCAUUGUAUUUUUAGGUCC | 201 | MIMAT0003252 |
| hsa-miR-668 | UGUCACUCGGCUCGGCCCACUAC | 202 | MIMAT0003881 |
| hsa-miR-18a | UAAGGUGCAUCUAGUGCAGAUAG | 203 | MIMAT0000072 |
| hsa-miR-29b-2* | CUGGUUUCACAUGGUGGCUUAG | 204 | MIMAT0004515 |
| hsa-let-7b* | CUAUACAACCUACUGCCUUCCC | 205 | MIMAT0004482 |
| hsa-miR-629* | GUUCUCCCAACGUAAGCCCAGC | 206 | MIMAT0003298 |
| hsa-miR-1243 | AACUGGAUCAAUUAUAGGAGUG | 207 | MIMAT0005894 |
| hsa-miR-933 | UGUGCGCAGGGAGACCUCUCCC | 208 | MIMAT0004976 |
| hsa-miR-181c* | AACCAUCGACCGUUGAGUGGAC | 209 | MIMAT0004559 |
| hsa-miR-505 | CGUCAACACUUGCUGGUUUCCU | 210 | MIMAT0002876 |
| hsa-miR-562 | AAAGUAGCUGUACCAUUUGC | 211 | MIMAT0003226 |
| hsa-miR-573 | CUGAAGUGAUGUGUAACUGAUCAG | 212 | MIMAT0003238 |
| hsa-let-7a* | CUAUACAAUCUACUGUCUUUC | 213 | MIMAT0004481 |
| hsa-miR-376b | AUCAUAGAGGAAAAUCCAUGUU | 214 | MIMAT0002172 |
| hsa-miR-27b* | AGAGCUUAGCUGAUUGGUGAAC | 215 | MIMAT0004588 |
| hsa-miR-891a | UGCAACGAACCUGAGCCACUGA | 216 | MIMAT0004902 |
| hsa-miR-532-5p | CAUGCCUUGAGUGUAGGACCGU | 217 | MIMAT0002888 |
| hsa-miR-590-5p | GAGCUUAUUCAUAAAAGUGCAG | 218 | MIMAT0003258 |
| hsa-miR-302b | UAAGUGCUUCCAUGUUUUAGUAG | 219 | MIMAT0000715 |
| hsa-miR-589* | UCAGAACAAAUGCCGGUUCCCAGA | 220 | MIMAT0003256 |
| hsa-miR-558 | UGAGCUGCUGUACCAAAAU | 221 | MIMAT0003222 |
| hsa-miR-193b | AACUGGCCCUCAAAGUCCCGCU | 222 | MIMAT0002819 |
| hsa-miR-126 | UCGUACCGUGAGUAAUAAUGCG | 223 | MIMAT0000445 |
| hsa-miR-634 | AACCAGCACCCCAACUUUGGAC | 224 | MIMAT0003304 |
| hsa-miR-1245 | AAGUGAUCUAAAGGCCUACAU | 225 | MIMAT0005897 |
| hsa-miR-21 | UAGCUUAUCAGACUGAUGUUGA | 226 | MIMAT0000076 |
| hsa-miR-875-3p | CCUGGAAACACUGAGGUUGUG | 227 | MIMAT0004923 |
| hsa-miR-556-3p | AUAUUACCAUUAGCUCAUCUUU | 228 | MIMAT0004793 |
| hsa-miR-650 | AGGAGGCAGCGCUCUCAGGAC | 229 | MIMAT0003320 |
| hsa-miR-638 | AGGGAUCGCGGGCGGGUGGCGGC | 230 | MIMAT0003308 |
| | CU | | |
| hsa-miR-518a-3p | GAAAGCGCUUCCCUUUGCUGGA | 231 | MIMAT0002863 |
| hsa-miR-31 | AGGCAAGAUGCUGGCAUAGCU | 232 | MIMAT0000089 |
| hsa-miR-1258 | AGUUAGGAUUAGGUCGUGGAA | 233 | MIMAT0005909 |
| hsa-miR-767-5p | UGCACCAUGGUUGUCUGAGCAUG | 234 | MIMAT0003882 |
| hsa-miR-188-5p | CAUCCCUUGCAUGGUGGAGGG | 235 | MIMAT0000457 |
| hsa-miR-556-5p | GAUGAGCUCAUUGUAAUAUGAG | 236 | MIMAT0003220 |
| hsa-miR-361-5p | UUAUCAGAAUCUCCAGGGGUAC | 237 | MIMAT0000703 |
| hsa-miR-1272 | GAUGAUGAUGGCAGCAAAUUCUGA | 238 | MIMAT0005925 |
| | AA | | |
| hsa-miR-15b | UAGCAGCACAUCAUGGUUUACA | 239 | MIMAT0000417 |
| hsa-miR-1244 | AAGUAGUUGGUUUGUAUGAGAUGG | 240 | MIMAT0005896 |
| | UU | | |
| hsa-miR-767-3p | UCUGCUCAUACCCCAUGGUUUCU | 241 | MIMAT0003883 |
| hsa-let-7i* | CUGCGCAAGCUACUGCCUUGCU | 242 | MIMAT0004585 |
| hsa-miR-920 | GGGGAGCUGUGGAAGCAGUA | 243 | MIMAT0004970 |
| hsa-miR-587 | UUUCCAUAGGUGAUGAGUCAC | 244 | MIMAT0003253 |
| hsa-miR-340* | UCCGUCUCAGUUACUUUAUAGC | 245 | MIMAT0000750 |
| hsa-miR-875-5p | UAUACCUCAGUUUUAUCAGGUG | 246 | MIMAT0004922 |
| hsa-miR-27b | UUCACAGUGGCUAAGUUCUGC | 247 | MIMAT0000419 |
| hsa-miR-1248 | ACCUUCUUGUAUAAGCACUGUGCU | 248 | MIMAT0005900 |
| | AAA | | |
| hsa-miR-582-5p | UUACAGUUGUUCAACCAGUUACU | 249 | MIMAT0003247 |
| hsa-miR-22* | AGUUCUUCAGUGGCAAGCUUUA | 250 | MIMAT0004495 |
| hsa-miR-223 | UGUCAGUUUGUCAAAUACCCCA | 251 | MIMAT0000280 |
| hsa-miR-548c-5p | AAAAGUAAUUGCGGUUUUUGCC | 252 | MIMAT0004806 |
| hsa-miR-92a | UAUUGCACUUGUCCCGGCCUGU | 253 | MIMAT0000092 |
| hsa-miR-526b | CUCUUGAGGGAAGCACUUUCUGU | 254 | MIMAT0002835 |
| hsa-miR-24 | UGGCUCAGUUCAGCAGGAACAG | 255 | MIMAT0000080 |
| hsa-miR-29b-1* | GCUGGUUUCAUAUGGUGGUUUAGA | 256 | MIMAT0004514 |
| hsa-miR-526b* | GAAAGUGCUUCCUUUUAGAGGC | 257 | MIMAT0002836 |
| hsa-miR-877* | UCCUCUUCUCCCUCCUCCCAG | 258 | MIMAT0004950 |
| hsa-miR-182 | UUUGGCAAUGGUAGAACUCACACU | 259 | MIMAT0000259 |
| hsa-miR-133a | UUUGGUCCCCUUCAACCAGCUG | 260 | MIMAT0000427 |
| hsa-miR-124* | CGUGUUCACAGCGGACCUUGAU | 261 | MIMAT0004591 |
| hsa-miR-1236 | CCUCUUCCCCUUGUCUCUCCAG | 262 | MIMAT0005591 |
| hsa-miR-578 | CUUCUUGUGCUCUAGGAUUGU | 263 | MIMAT0003243 |
| hsa-miR-769-5p | UGAGACCUCUGGGUUCUGAGCU | 264 | MIMAT0003886 |
| hsa-miR-599 | GUUGUGUCAGUUUAUCAAAC | 265 | MIMAT0003267 |
| hsa-miR-192* | CUGCCAAUUCCAUAGGUCACAG | 266 | MIMAT0004543 |
| hsa-miR-614 | GAACGCCUGUUCUUGCCAGGUGG | 267 | MIMAT0003282 |
| hsa-miR-643 | ACUUGUAUGCUAGCUCAGGUAG | 268 | MIMAT0003313 |
| hsa-miR-541 | UGGUGGGCACAGAAUCUGGACU | 269 | MIMAT0004920 |
| hsa-miR-92a-2* | GGGUGGGGAUUUGUUGCAUUAC | 270 | MIMAT0004508 |
| hsa-miR-323-3p | CACAUUACACGGUCGACCUCU | 271 | MIMAT0000755 |
| hsa-miR-454* | ACCCUAUCAAUAUUGUCUCUGC | 272 | MIMAT0003884 |
| hsa-miR-518c* | UCUCUGGAGGGAAGCACUUUCUG | 273 | MIMAT0002847 |
| hsa-miR-921 | CUAGUGAGGGACAGAACCAGGAUU | 274 | MIMAT0004971 |
| | C | | |
| hsa-miR-566 | GGGCGCCUGUGAUCCCAAC | 275 | MIMAT0003230 |
| hsa-miR-520f | AAGUGCUUCCUUUUAGAGGGUU | 276 | MIMAT0002830 |
| hsa-miR-663 | AGGCGGGGCGCCGCGGGACCGC | 277 | MIMAT0003326 |
| hsa-miR-203 | GUGAAAUGUUUAGGACCACUAG | 278 | MIMAT0000264 |
| hsa-miR-608 | AGGGGUGGUGUUGGGACAGCUCC | 279 | MIMAT0003276 |
| | GU | | |
| hsa-miR-513c | UUCUCAAGGAGGUGUCGUUUAU | 280 | MIMAT0005789 |
| hsa-miR-95 | UUCAACGGGUAUUUAUUGAGCA | 281 | MIMAT0000094 |
| hsa-miR-216b | AAAUCUCUGCAGGCAAAUGUGA | 282 | MIMAT0004959 |
| hsa-let-7d* | CUAUACGACCUGCUGCCUUUCU | 283 | MIMAT0004484 |
| hsa-miR-142-3p | UGUAGUGUUUCCUACUUUAUGGA | 284 | MIMAT0000434 |
| hsa-miR-20a | UAAAGUGCUUAUAGUGCAGGUAG | 285 | MIMAT0000075 |
| hsa-miR-505* | GGGAGCCAGGAAGUAUUGAUGU | 286 | MIMAT0004776 |
| hsa-miR-152 | UCAGUGCAUGACAGAACUUGG | 287 | MIMAT0000438 |
| hsa-miR-125b-2* | UCACAAGUCAGGCUCUUGGGAC | 288 | MIMAT0004603 |
| hsa-miR-379 | UGGUAGACUAUGGAACGUAGG | 289 | MIMAT0000733 |
| hsa-miR-20b | CAAAGUGCUCAUAGUGCAGGUAG | 290 | MIMAT0001413 |
| hsa-miR-636 | UGUGCUUGCUCGUCCCGCCCGCA | 291 | MIMAT0003306 |
| hsa-miR-371-3p | AAGUGCCGCCAUCUUUUGAGUGU | 292 | MIMAT0000723 |
| hsa-miR-302e | UAAGUGCUUCCAUGCUU | 293 | MIMAT0005931 |
| hsa-miR-452 | AACUGUUUGCAGAGGAAACUGA | 294 | MIMAT0001635 |
| hsa-miR-21* | CAACACCAGUCGAUGGGCUGU | 295 | MIMAT0004494 |
| hsa-miR-324-3p | ACUGCCCCAGGUGCUGCUGG | 296 | MIMAT0000762 |
| hsa-miR-140-3p | UACCACAGGGUAGAACCACGG | 297 | MIMAT0004597 |
| hsa-miR-516b*, | UGCUUCCUUUCAGAGGGU | 298 | MIMAT0002860 |
| hsa-miR-516a-3p, | | | |
| hsa-miR-191 | CAACGGAAUCCCAAAAGCAGCUG | 299 | MIMAT0000440 |
| hsa-miR-621 | GGCUAGCAACAGCGCUUACCU | 300 | MIMAT0003290 |
| hsa-miR-155 | UUAAUGCUAAUCGUGAUAGGGGU | 301 | MIMAT0000646 |
| hsa-miR-16-2* | CCAAUAUUACUGUGCUGCUUUA | 302 | MIMAT0004518 |
| hsa-miR-19b-1* | AGUUUUGCAGGUUUGCAUCCAGC | 303 | MIMAT0004491 |
| hsa-miR-302d | UAAGUGCUUCCAUGUUUGAGUGU | 304 | MIMAT0000718 |
| hsa-miR-631 | AGACCUGGCCCAGACCUCAGC | 305 | MIMAT0003300 |
| hsa-miR-550* | UGUCUUACUCCCUCAGGCACAU | 306 | MIMAT0003257 |
| hsa-miR-222* | CUCAGUAGCCAGUGUAGAUCCU | 307 | MIMAT0004569 |
| hsa-let-7g* | CUGUACAGGCCACUGCCUUGC | 308 | MIMAT0004584 |
| hsa-miR-602 | GACACGGGCGACAGCUGCGGCCC | 309 | MIMAT0003270 |
| hsa-miR-130b | CAGUGCAAUGAUGAAAGGGCAU | 310 | MIMAT0000691 |
| hsa-miR-34a* | CAAUCAGCAAGUAUACUGCCCU | 311 | M1MAT0004557 |
| hsa-miR-124 | UAAGGCACGCGGUGAAUGCC | 312 | MIMAT0000422 |
| hsa-miR-598 | UACGUCAUCGUUGUCAUCGUCA | 313 | MIMAT0003266 |
| hsa-miR-149 | UCUGGCUCCGUGUCUUCACUCCC | 314 | MIMAT0000450 |
| hsa-miR-28-5p | AAGGAGCUCACAGUCUAUUGAG | 315 | MIMAT0000085 |
| hsa-let-7f-1* | CUAUACAAUCUAUUGCCUUCCC | 316 | MIMAT0004486 |
| hsa-miR-19b-2* | AGUUUUGCAGGUUUGCAUUUCA | 317 | MIMAT0004492 |
| hsa-miR-135a | UAUGGCUUUUUAUUCCUAUGUGA | 318 | MIMAT0000428 |
| hsa-let-7a | UGAGGUAGUAGGUUGUAUAGUU | 319 | MIMAT0000062 |
| hsa-miR-106b | UAAAGUGCUGACAGUGCAGAU | 320 | MIMAT0000680 |
| hsa-miR-2110 | UUGGGGAAACGGCCGCUGAGUG | 321 | MIMAT0010133 |
| hsa-miR-130a* | UUCACAUUGUGCUACUGUCUGC | 322 | MIMAT0004593 |
| hsa-miR-1184 | CCUGCAGCGACUUGAUGGCUUCC | 323 | MIMAT0005829 |
| hsa-miR-551a | GCGACCCACUCUUGGUUUCCA | 324 | MIMAT0003214 |
| hsa-miR-519b-3p | AAAGUGCAUCCUUUUAGAGGUU | 325 | MIMAT0002837 |
| hsa-miR-210 | CUGUGCGUGUGACAGCGGCUGA | 326 | MIMAT0000267 |
| hsa-miR-503 | UAGCAGCGGGAACAGUUCUGCAG | 327 | MIMAT0002874 |
| hsa-miR-549 | UGACAACUAUGGAUGAGCUCU | 328 | MIMAT0003333 |
| hsa-miR-517* | CCUCUAGAUGGAAGCACUGUCU | 329 | MIMAT0002851 |
| hsa-miR-425 | AAUGACACGAUCACUCCCGUUGA | 330 | MIMAT0003393 |
| hsa-miR-153 | UUGCAUAGUCACAAAAGUGAUC | 331 | MIMAT0000439 |
| hsa-miR-125a-5p | UCCCUGAGACCCUUUAACCUGUGA | 332 | MIMAT0000443 |
| hsa-miR-520a-5p | CUCCAGAGGGAAGUACUUUCU | 333 | MIMAT0002833 |
| hsa-miR-198 | GGUCCAGAGGGGAGAUAGGUUC | 334 | MIMAT0000228 |
| hsa-miR-571 | UGAGUUGGCCAUCUGAGUGAG | 335 | MIMAT0003236 |
| hsa-miR-30b | UGUAAACAUCCUACACUCAGCU | 336 | MIMAT0000420 |
| hsa-miR-1 | UGGAAUGUAAAGAAGUAUGUAU | 337 | MIMAT0000416 |
| hsa-miR-379* | UAUGUAACAUGGUCCACUAACU | 338 | MIMAT0004690 |
| hsa-miR-557 | GUUUGCACGGGUGGGCCUUGUCU | 339 | MIMAT0003221 |
| hsa-miR-378* | CUCCUGACUCCAGGUCCUGUGU | 340 | MIMAT0000731 |
| hsa-miR-490-3p | CAACCUGGAGGACUCCAUGCUG | 341 | MIMAT0002806 |
| hsa-miR-510 | UACUCAGGAGAGUGGCAAUCAC | 342 | MIMAT0002882 |
| hsa-miR-1201 | AGCCUGAUUAAACACAUGCUCUGA | 343 | MIMAT0005864 |
| hsa-miR-1271 | CUUGGCACCUAGCAAGCACUCA | 344 | MIMAT0005796 |
| hsa-miR-200a* | CAUCUUACCGGACAGUGCUGGA | 345 | MIMAT0001620 |
| hsa-miR-758 | UUUGUGACCUGGUCCACUAACC | 346 | MIMAT0003879 |
| hsa-miR-497 | CAGCAGCACACUGUGGUUUGU | 347 | MIMAT0002820 |
| hsa-miR-525-5p | CUCCAGAGGGAUGCACUUUCU | 348 | MIMAT0002838 |
| hsa-miR-220c | ACACAGGGCUGUUGUGAAGACU | 349 | MIMAT0004915 |
| hsa-miR-24-1* | UGCCUACUGAGCUGAUAUCAGU | 350 | MIMAT0000079 |
| hsa-miR-409-3p | GAAUGUUGCUCGGUGAACCCCU | 351 | MIMAT0001639 |
| hsa-let-7f | UGAGGUAGUAGAUUGUAUAGUU | 352 | MIMAT0000067 |
| hsa-miR-675* | CUGUAUGCCCUCACCGCUCA | 353 | MIMAT0006790 |
| hsa-miR-25 | CAUUGCACUUGUCUCGGUCUGA | 354 | MIMAT0000081 |
| hsa-miR-375 | UUUGUUCGUUCGGCUCGCGUGA | 355 | MIMAT0000728 |
| hsa-miR-455-5p | UAUGUGCCUUUGGACUACAUCG | 356 | MIMAT0003150 |
| hsa-miR-328 | CUGGCCCUCUCUGCCCUUCCGU | 357 | MIMAT0000752 |
| hsa-miR-574-3p | CACGCUCAUGCACACACCCACA | 358 | MIMAT0003239 |
| hsa-miR-671-5p | AGGAAGCCCUGGAGGGGCUGGAG | 359 | MIMAT0003880 |
| hsa-miR-99b | CACCCGUAGAACCGACCUUGCG | 360 | MIMAT0000689 |
| hsa-miR-147b | GUGUGCGGAAAUGCUUCUGCUA | 361 | MIMAT0004928 |
| hsa-miR-450b-3p | UUGGGAUCAUUUUGCAUCCAUA | 362 | MIMAT0004910 |
| hsa-miR-629 | UGGGUUUACGUUGGGAGAACU | 363 | MIMAT0004810 |
| hsa-miR-663b | GGUGGCCCGGCCGUGCCUGAGG | 364 | MIMAT0005867 |
| hsa-miR-330-5p | UCUCUGGGCCUGUGUCUUAGGC | 365 | MIMAT0004693 |
| hsa-miR-34c-3p | AAUCACUAACCACACGGCCAGG | 366 | MIMAT0004677 |
| hsa-miR-146b-3p | UGCCCUGUGGACUCAGUUCUGG | 367 | MIMAT0004766 |
| hsa-miR-592 | UUGUGUCAAUAUGCGAUGAUGU | 368 | MIMAT0003260 |
| hsa-miR-30d | UGUAAACAUCCCCGACUGGAAG | 369 | MIMAT0000245 |
| hsa-miR-555 | AGGGUAAGCUGAACCUCUGAU | 370 | MIMAT0003219 |
| hsa-miR-23a | AUCACAUUGCCAGGGAUUUCC | 371 | MIMAT0000078 |
| hsa-miR-101* | CAGUUAUCACAGUGCUGAUGCU | 372 | MIMAT0004513 |
| hsa-miR-197 | UUCACCACCUUCUCCACCCAGC | 373 | MIMAT0000227 |
| hsa-miR-487a | AAUCAUACAGGGACAUCCAGUU | 374 | MIMAT0002178 |
| hsa-miR-512-3p | AAGUGCUGUCAUAGCUGAGGUC | 375 | MIMAT0002823 |
| hsa-miR-520h | ACAAAGUGCUUCCCUUUAGAGU | 376 | MIMAT0002867 |
| hsa-miR-92b | UAUUGCACUCGUCCCGGCCUCC | 377 | MIMAT0003218 |
| hsa-miR-138 | AGCUGGUGUUGUGAAUCAGGCCG | 378 | MIMAT0000430 |
| hsa-miR-196a | UAGGUAGUUUCAUGUUGUUGGG | 379 | MIMAT0000226 |
| hsa-miR-652 | AAUGGCGCCACUAGGGUUGUG | 380 | MIMAT0003322 |
| hsa-let-7a-2* | CUGUACAGCCUCCUAGCUUUCC | 381 | MIMAT0010195 |
| hsa-miR-105 | UCAAAUGCUCAGACUCCUGUGGU | 382 | MIMAT0000102 |
| hsa-miR-301b | CAGUGCAAUGAUAUUGUCAAAGC | 383 | MIMAT0004958 |
| hsa-miR-337-5p | GAACGGCUUCAUACAGGAGUU | 384 | MIMAT0004695 |
| hsa-miR-630 | AGUAUUCUGUACCAGGGAAGGU | 385 | MIMAT0003299 |
| hsa-miR-296-3p | GAGGGUUGGGUGGAGGCUCUCC | 386 | MIMAT0004679 |
| hsa-let-7i | UGAGGUAGUAGUUUGUGCUGUU | 387 | MIMAT0000415 |
| hsa-miR-489 | GUGACAUCACAUAUACGGCAGC | 388 | MIMAT0002805 |
| hsa-miR-504 | AGACCCUGGUCUGCACUCUAUC | 389 | MIMAT0002875 |
| hsa-miR-15b* | CGAAUCAUUAUUUGCUGCUCUA | 390 | MIMAT0004586 |
| hsa-miR-147 | GUGUGUGGAAAUGCUUCUGC | 391 | MIMAT0000251 |
| hsa-miR-376a* | GUAGAUUCUCCUUCUAUGAGUA | 392 | MIMAT0003386 |
| hsa-miR-125b-1* | ACGGGUUAGGCUCUUGGGAGCU | 393 | MIMAT0004592 |
| hsa-miR-146a* | CCUCUGAAAUUCAGUUCUUCAG | 394 | MIMAT0004608 |
| hsa-miR-187* | GGCUACAACACAGGACCCGGGC | 395 | MIMAT0004561 |
| hsa-miR-302c | UAAGUGCUUCCAUGUUUCAGUGG | 396 | MIMAT0000717 |
| hsa-miR-520b | AAAGUGCUUCCUUUUAGAGGG | 397 | MIMAT0002843 |
| hsa-miR-518b | CAAAGCGCUCCCCUUUAGAGGU | 398 | MIMAT0002844 |
| hsa-miR-886-5p | CGGGUCGGAGUUAGCUCAAGCGG | 399 | MIMAT0004905 |
| hsa-miR-34c-5p | AGGCAGUGUAGUUAGCUGAUUGC | 400 | MIMAT0000686 |
| hsa-miR-16 | UAGCAGCACGUAAAUAUUGGCG | 401 | MIMAT0000069 |
| hsa-miR-30e* | CUUUCAGUCGGAUGUUUACAGC | 402 | MIMAT0000693 |
| hsa-miR-641 | AAAGACAUAGGAUAGAGUCACCUC | 403 | MIMAT0003311 |
| hsa-miR-188-3p | CUCCCACAUGCAGGGUUUGCA | 404 | MIMAT0004613 |
| hsa-miR-1203 | CCCGGAGCCAGGAUGCAGCUC | 405 | MIMAT0005866 |
| hsa-miR-92b* | AGGGACGGGACGCGGUGCAGUG | 406 | MIMAT0004792 |
| hsa-miR-548a-5p | AAAAGUAAUUGCGAGUUUUACC | 407 | MIMAT0004803 |
| hsa-miR-96 | UUUGGCACUAGCACAUUUUUGCU | 408 | MIMAT0000095 |
| hsa-miR-23b | AUCACAUUGCCAGGGAUUACC | 409 | MIMAT0000418 |
| hsa-miR-219-1-3p | AGAGUUGAGUCUGGACGUCCCG | 410 | MIMAT0004567 |
| hsa-miR-1266 | CCUCAGGGCUGUAGAACAGGGCU | 411 | MIMAT0005920 |
| hsa-miR-548j | AAAAGUAAUUGCGGUCUUUGGU | 412 | MIMAT0005875 |
| hsa-miR-495 | AAACAAACAUGGUGCACUUCUU | 413 | MIMAT0002817 |
| hsa-miR-331-5p | CUAGGUAUGGUCCCAGGGAUCC | 414 | MIMAT0004700 |
| hsa-miR-34b* | UAGGCAGUGUCAUUAGCUGAUUG | 415 | MIMAT0000685 |
| hsa-miR-500 | UAAUCCUUGCUACCUGGGUGAGA | 416 | MIMAT0004773 |
| hsa-miR-601 | UGGUCUAGGAUUGUUGGAGGAG | 417 | MIMAT0003269 |
| hsa-miR-135b* | AUGUAGGGCUAAAAGCCAUGGG | 418 | MIMAT0004698 |
| hsa-let-7e | UGAGGUAGGAGGUUGUAUAGUU | 419 | MIMAT0000066 |
| hsa-miR-876-3p | UGGUGGUUUACAAAGUAAUUCA | 420 | MIMAT0004925 |
| hsa-miR-29a* | ACUGAUUUCUUUUGGUGUUCAG | 421 | MIMAT0004503 |
| hsa-miR-515-5p | UUCUCCAAAAGAAAGCACUUUCUG | 422 | MIMAT0002826 |
| hsa-miR-96* | AAUCAUGUGCAGUGCCAAUAUG | 423 | MIMAT0004510 |
| hsa-miR-411* | UAUGUAACACGGUCCACUAACC | 424 | MIMAT0004813 |
| hsa-miR-15a* | CAGGCCAUAUUGUGCUGCCUCA | 425 | MIMAT0004488 |
| hsa-miR-296-5p | AGGGCCCCCCCUCAAUCCUGU | 426 | MIMAT0000690 |
| hsa-miR-122* | AACGCCAUUAUCACACUAAAUA | 427 | MIMAT0004590 |
| hsa-miR-499-3p | AACAUCACAGCAAGUCUGUGCU | 428 | MIMAT0004772 |
| hsa-miR-654-5p | UGGUGGGCCGCAGAACAUGUGC | 429 | MIMAT0003330 |
| hsa-miR-942 | UCUUCUCUGUUUUGGCCAUGUG | 430 | MIMAT0004985 |
| hsa-miR-496 | UGAGUAUUACAUGGCCAAUCUC | 431 | MIMAT0002818 |
| hsa-miR-376c | AACAUAGAGGAAAUUCCACGU | 432 | MIMAT0000720 |
| hsa-miR-106a* | CUGCAAUGUAAGCACUUCUUAC | 433 | MIMAT0004517 |
| hsa-let-7c | UGAGGUAGUAGGUUGUAUGGUU | 434 | MIMAT0000064 |
| hsa-miR-615-5p | GGGGGUCCCCGGUGCUCGGAUC | 435 | MIMAT0004804 |
| hsa-miR-125a-3p | ACAGGUGAGGUUCUUGGGAGCC | 436 | MIMAT0004602 |
| hsa-miR-543 | AAACAUUCGCGGUGCACUUCUU | 437 | MIMAT0004954 |
| hsa-miR-484 | UCAGGCUCAGUCCCCUCCCGAU | 438 | MIMAT0002174 |
| hsa-miR-502-5p | AUCCUUGCUAUCUGGGUGCUA | 439 | MIMAT0002873 |
| hsa-miR-19b | UGUGCAAAUCCAUGCAAAACUGA | 440 | MIMAT0000074 |
| hsa-miR-523 | GAACGCGCUUCCCUAUAGAGGGU | 441 | MIMAT0002840 |
| hsa-miR-615-3p | UCCGAGCCUGGGUCUCCCUCUU | 442 | MIMAT0003283 |
| hsa-miR-564 | AGGCACGGUGUCAGCAGGC | 443 | MIMAT0003228 |
| hsa-miR-1269 | CUGGACUGAGCCGUGCUACUGG | 444 | MIMAT0005923 |
| hsa-miR-130b* | ACUCUUUCCCUGUUGCACUAC | 445 | MIMAT0004680 |
| hsa-miR-30a* | CUUUCAGUCGGAUGUUUGCAGC | 446 | MIMAT0000088 |
| hsa-miR-509-3p | UGAUUGGUACGUCUGUGGGUAG | 447 | MIMAT0002881 |
| hsa-miR-412 | ACUUCACCUGGUCCACUAGCCGU | 448 | MIMAT0002170 |
| hsa-miR-526a, | CUCUAGAGGGAAGCACUUUCUG | 449 | MIMAT0002845 |
| hsa-miR-518d-5p & | | | |
| hsa-miR-520c-5p | | | |
| hsa-miR-33b* | CAGUGCCUCGGCAGUGCAGCCC | 450 | MIMAT0004811 |
| hsa-miR-877 | GUAGAGGAGAUGGCGCAGGG | 451 | MIMAT0004949 |
| hsa-miR-325 | CCUAGUAGGUGUCCAGUAAGUGU | 452 | MIMAT0000771 |
| hsa-miR-125b | UCCCUGAGACCCUAACUUGUGA | 453 | MIMAT0000423 |
| hsa-miR-1182 | GAGGGUCUUGGGAGGGAUGUGAC | 454 | MIMAT0005827 |
| hsa-miR-107 | AGCAGCAUUGUACAGGGCUAUCA | 455 | MIMAT0000104 |
| hsa-miR-488 | UUGAAAGGCUAUUUCUUGGUC | 456 | MIMAT0004763 |
| hsa-miR-93* | ACUGCUGAGCUAGCACUUCCCG | 457 | MIMAT0004509 |
| hsa-miR-516a-5p | UUCUCGAGGAAAGAAGCACUUUC | 458 | MIMAT0004770 |
| hsa-miR-887 | GUGAACGGGCGCCAUCCCGAGG | 459 | MIMAT0004951 |
| hsa-miR-885-5p | UCCAUUACACUACCCUGCCUCU | 460 | MIMAT0004947 |
| hsa-miR-888* | GACUGACACCUCUUUGGGUGAA | 461 | MIMAT0004917 |
| hsa-miR-185 | UGGAGAGAAAGGCAGUUCCUGA | 462 | MIMAT0000455 |
| hsa-miR-138-2* | GCUAUUUCACGACACCAGGGUU | 463 | MIMAT0004596 |
| hsa-miR-922 | GCAGCAGAGAAUAGGACUACGUC | 464 | MIMAT0004972 |
| hsa-miR-200c* | CGUCUUACCCAGCAGUGUUUGG | 465 | MIMAT0004657 |
| hsa-miR-508-3p | UGAUUGUAGCCUUUUGGAGUAGA | 466 | MIMAT0002880 |
| hsa-miR-449a | UGGCAGUGUAUUGUUAGCUGGU | 467 | MIMAT0001541 |
| hsa-miR-200c | UAAUACUGCCGGGUAAUGAUGGA | 468 | MIMAT0000617 |
| hsa-miR-145 | GUCCAGUUUUCCCAGGAAUCCCU | 469 | MIMAT0000437 |
| hsa-miR-218 | UUGUGCUUGAUCUAACCAUGU | 470 | MIMAT0000275 |
| hsa-miR-548b-3p | CAAGAACCUCAGUUGCUUUUGU | 471 | MIMAT0003254 |
| hsa-miR-34a | UGGCAGUGUCUUAGCUGGUUGU | 472 | MIMAT0000255 |
| hsa-miR-205 | UCCUUCAUUCCACCGGAGUCUG | 473 | MIMAT0000266 |
| hsa-miR-423-3p | AGCUCGGUCUGAGGCCCCUCAGU | 474 | MIMAT0001340 |
| hsa-miR-487b | AAUCGUACAGGGUCAUCCACUU | 475 | MIMAT0003180 |
| hsa-miR-708 | AAGGAGCUUACAAUCUAGCUGGG | 476 | MIMAT0004926 |
| hsa-miR-519e | AAGUGCCUCCUUUUAGAGUGUU | 477 | MIMAT0002829 |
| hsa-miR-610 | UGAGCUAAAUGUGUGCUGGGA | 478 | MIMAT0003278 |
| hsa-miR-371-5p | ACUCAAACUGUGGGGGCACU | 479 | MIMAT0004687 |
| hsa-miR-199a-5p | CCCAGUGUUCAGACUACCUGUUC | 480 | MIMAT0000231 |
| hsa-miR-488* | CCCAGAUAAUGGCACUCUCAA | 481 | MIMAT0002804 |
| hsa-miR-1260 | AUCCCACCUCUGCCACCA | 482 | MIMAT0005911 |
| hsa-miR-520c-3p | AAAGUGCUUCCUUUUAGAGGGU | 483 | MIMAT0002846 |
| hsa-miR-616* | ACUCAAAACCCUUCAGUGACUU | 484 | MIMAT0003284 |
| hsa-miR-766 | ACUCCAGCCCCACAGCCUCAGC | 485 | MIMAT0003888 |
| hsa-miR-141* | CAUCUUCCAGUACAGUGUUGGA | 486 | MIMAT0004598 |
| hsa-miR-622 | ACAGUCUGCUGAGGUUGGAGC | 487 | MIMAT0003291 |
| hsa-miR-17* | ACUGCAGUGAAGGCACUUGUAG | 488 | MIMAT0000071 |
| hsa-miR-509-3-5p | UACUGCAGACGUGGCAAUCAUG | 489 | MIMAT0004975 |
| hsa-miR-141 | UAACACUGUCUGGUAAAGAUGG | 490 | MIMAT0000432 |
| hsa-miR-580 | UUGAGAAUGAUGAAUCAUUAGG | 491 | MIMAT0003245 |
| hsa-miR-517a | AUCGUGCAUCCCUUUAGAGUGU | 492 | MIMAT0002852 |
| hsa-miR-204 | UUCCCUUUGUCAUCCUAUGCCU | 493 | MIMAT0000265 |
| hsa-miR-376a | AUCAUAGAGGAAAAUCCACGU | 494 | MIMAT0000729 |
| hsa-miR-335* | UUUUUCAUUAUUGCUCCUGACC | 495 | MIMAT0004703 |
| hsa-miR-214 | ACAGCAGGCACAGACAGGCAGU | 496 | MIMAT0000271 |
| hsa-miR-342-3p | UCUCACACAGAAAUCGCACCCGU | 497 | MIMAT0000753 |
| hsa-miR-326 | CCUCUGGGCCCUUCCUCCAG | 498 | MIMAT0000756 |
| hsa-miR-9 | UCUUUGGUUAUCUAGCUGUAUGA | 499 | MIMAT0000441 |
| hsa-miR-10b* | ACAGAUUCGAUUCUAGGGGAAU | 500 | MIMAT0004556 |
| hsa-miR-23b* | UGGGUUCCUGGCAUGCUGAUUU | 501 | MIMAT0004587 |
| hsa-miR-342-5p | AGGGGUGCUAUCUGUGAUUGA | 502 | MIMAT0004694 |
| hsa-miR-449b | AGGCAGUGUAUUGUUAGCUGGC | 503 | MIMAT0003327 |
| hsa-miR-154 | UAGGUUAUCCGUGUUGCCUUCG | 504 | MIMAT0000452 |
| hsa-miR-450a | UUUUGCGAUGUGUUCCUAAUAU | 505 | MIMAT0001545 |
| hsa-miR-99a* | CAAGCUCGCUUCUAUGGGUCUG | 506 | MIMAT0004511 |
| hsa-miR-99a | AACCCGUAGAUCCGAUCUUGUG | 507 | MIMAT0000097 |
| hsa-miR-658 | GGCGGAGGGAAGUAGGUCCGUUG | 508 | MIMAT0003336 |
| | GU | | |
| hsa-miR-18a* | ACUGCCCUAAGUGCUCCUUCUGG | 509 | MIMAT0002891 |
| hsa-miR-320b | AAAAGCUGGGUUGAGAGGGCAA | 510 | MIMAT0005792 |
| hsa-miR-1253 | AGAGAAGAAGAUCAGCCUGCA | 511 | MIMAT0005904 |
| hsa-miR-1296 | UUAGGGCCCUGGCUCCAUCUCC | 512 | MIMAT0005794 |
| hsa-miR-876-5p | UGGAUUUCUUUGUGAAUCACCA | 513 | MIMAT0004924 |
| hsa-miR-744* | CUGUUGCCACUAACCUCAACCU | 514 | MIMAT0004946 |
| hsa-miR-223* | CGUGUAUUUGACAAGCUGAGUU | 515 | MIMAT0004570 |
| hsa-miR-181b | AACAUUCAUUGCUGUCGGUGGGU | 516 | MIMAT0000257 |
| hsa-miR-411 | UAGUAGACCGUAUAGCGUACG | 517 | MIMAT0003329 |
| hsa-miR-221 | AGCUACAUUGUCUGCUGGGUUUC | 518 | MIMAT0000278 |
| hsa-miR-640 | AUGAUCCAGGAACCUGCCUCU | 519 | MIMAT0003310 |
| hsa-miR-129-5p | CUUUUUGCGGUCUGGGCUUGC | 520 | MIMAT0000242 |
| hsa-miR-100* | CAAGCUUGUAUCUAUAGGUAUG | 521 | MIMAT0004512 |
| hsa-miR-199a-3p & | ACAGUAGUCUGCACAUUGGUUA | 522 | MIMAT0000232 |
| hsa-miR-199b-3p | | | |
| hsa-miR-1208 | UCACUGUUCAGACAGGCGGA | 523 | MIMAT0005873 |
| hsa-miR-346 | UGUCUGCCCGCAUGCCUGCCUCU | 524 | MIMAT0000773 |
| hsa-miR-506 | UAAGGCACCCUUCUGAGUAGA | 525 | MIMAT0002878 |
| hsa-miR-140-5p | CAGUGGUUUUACCCUAUGGUAG | 526 | MIMAT0000431 |
| hsa-miR-424* | CAAAACGUGAGGCGCUGCUAU | 527 | MIMAT0004749 |
| hsa-miR-632 | GUGUCUGCUUCCUGUGGGA | 528 | MIMAT0003302 |
| hsa-miR-1267 | CCUGUUGAAGUGUAAUCCCCA | 529 | MIMAT0005921 |
| hsa-miR-299-5p | UGGUUUACCGUCCCACAUACAU | 530 | MIMAT0002890 |
| hsa-miR-943 | CUGACUGUUGCCGUCCUCCAG | 531 | MIMAT0004986 |
| hsa-miR-646 | AAGCAGCUGCCUCUGAGGC | 532 | MIMAT0003316 |
| hsa-miR-517b | UCGUGCAUCCCUUUAGAGUGUU | 533 | MIMAT0002857 |
| hsa-miR-760 | CGGCUCUGGGUCUGUGGGGA | 534 | MIMAT0004957 |
| hsa-miR-593* | AGGCACCAGCCAGGCAUUGCUCAG | 535 | MIMAT0003261 |
| | C | | |
| hsa-miR-222 | AGCUACAUCUGGCUACUGGGU | 536 | MIMAT0000279 |
| hsa-miR-132* | ACCGUGGCUUUCGAUUGUUACU | 537 | MIMAT0004594 |
| hsa-miR-146b-5p | UGAGAACUGAAUUCCAUAGGCU | 538 | MIMAT0002809 |
| hsa-miR-518c | CAAAGCGCUUCUCUUUAGAGUGU | 539 | MIMAT0002848 |
| hsa-miR-196b | UAGGUAGUUUCCUGUUGUUGGG | 540 | MIMAT0001080 |
| hsa-miR-554 | GCUAGUCCUGACUCAGCCAGU | 541 | MIMAT0003217 |
| hsa-miR-493 | UGAAGGUCUACUGUGUGCCAGG | 542 | MIMAT0003161 |
| hsa-miR-516b | AUCUGGAGGUAAGAAGCACUUU | 543 | MIMAT0002859 |
| hsa-miR-23a* | GGGGUUCCUGGGGAUGGGAUUU | 544 | MIMAT0004496 |
| hsa-miR-92a-1* | AGGUUGGGAUCGGUUGCAAUGCU | 545 | MIMAT0004507 |
| hsa-miR-374b* | CUUAGCAGGUUGUAUUAUCAUU | 546 | MIMAT0004956 |
| hsa-miR-138-1* | GCUACUUCACAACACCAGGGCC | 547 | MIMAT0004607 |
| hsa-miR-106a | AAAAGUGCUUACAGUGCAGGUAG | 548 | MIMAT0000103 |
| hsa-miR-617 | AGACUUCCCAUUUGAAGGUGGC | 549 | MIMAT0003286 |
| hsa-let-7g | UGAGGUAGUAGUUUGUACAGUU | 550 | MIMAT0000414 |
| hsa-miR-181a | AACAUUCAACGCUGUCGGUGAGU | 551 | MIMAT0000256 |
| hsa-miR-431* | CAGGUCGUCUUGCAGGGCUUCU | 552 | MIMAT0004757 |
| hsa-miR-584 | UUAUGGUUUGCCUGGGACUGAG | 553 | MIMAT0003249 |
| hsa-miR-20b* | ACUGUAGUAUGGGCACUUCCAG | 554 | MIMAT0004752 |
| hsa-miR-143* | GGUGCAGUGCUGCAUCUCUGGU | 555 | MIMAT0004599 |
| hsa-miR-886-3p | CGCGGGUGCUUACUGACCCUU | 556 | MIMAT0004906 |
| hsa-let-7c* | UAGAGUUACACCCUGGGAGUUA | 557 | MIMAT0004483 |
| hsa-miR-941 | CACCCGGCUGUGUGCACAUGUGC | 558 | MIMAT0004984 |
| hsa-miR-214* | UGCCUGUCUACACUUGCUGUGC | 559 | MIMAT0004564 |
| hsa-miR-151-3p | CUAGACUGAAGCUCCUUGAGG | 560 | MIMAT0000757 |
| hsa-miR-1468 | CUCCGUUUGCCUGUUUCGCUG | 561 | MIMAT0006789 |
| hsa-miR-639 | AUCGCUGCGGUUGCGAGCGCUGU | 562 | MIMAT0003309 |
| hsa-miR-494 | UGAAACAUACACGGGAAACCUC | 563 | MIMAT0002816 |
| hsa-miR-183* | GUGAAUUACCGAAGGGCCAUAA | 564 | MIMAT0004560 |
| hsa-miR-7-2* | CAACAAAUCCCAGUCUACCUAA | 565 | MIMAT0004554 |
| hsa-miR-454 | UAGUGCAAUAUUGCUUAUAGGGU | 566 | MIMAT0003885 |
| hsa-miR-548o | CCAAAACUGCAGUUACUUUUGC | 567 | MIMAT0005919 |
| hsa-miR-126* | CAUUAUUACUUUUGGUACGCG | 568 | MIMAT0000444 |
| hsa-miR-938 | UGCCCUUAAAGGUGAACCCAGU | 569 | MIMAT0004981 |
| hsa-miR-380 | UAUGUAAUAUGGUCCACAUCUU | 570 | MIMAT0000735 |
| hsa-miR-1908 | CGGCGGGGACGGCGAUUGGUC | 571 | MIMAT0007881 |
| hsa-miR-345 | GCUGACUCCUAGUCCAGGGCUC | 572 | MIMAT0000772 |
| hsa-miR-548h | AAAAGUAAUCGCGGUUUUUGUC | 573 | MIMAT0005928 |
| hsa-miR-193a-3p | AACUGGCCUACAAAGUCCCAGU | 574 | MIMAT0000459 |
| hsa-miR-7 | UGGAAGACUAGUGAUUUUGUUGU | 575 | MIMAT0000252 |
| hsa-miR-423-5p | UGAGGGGCAGAGAGCGAGACUUU | 576 | MIMAT0004748 |
| hsa-miR-1259 | AUAUAUGAUGACUUAGCUUUU | 577 | MIMAT0005910 |
| hsa-miR-1911 | UGAGUACCGCCAUGUCUGUUGGG | 578 | MIMAT0007885 |
| hsa-miR-605 | UAAAUCCCAUGGUGCCUUCUCCU | 579 | MIMAT0003273 |
| hsa-miR-513a-3p | UAAAUUUCACCUUUCUGAGAAGG | 580 | MIMAT0004777 |
| hsa-miR-215 | AUGACCUAUGAAUUGACAGAC | 581 | MIMAT0000272 |
| hsa-miR-1911* | CACCAGGCAUUGUGGUCUCC | 582 | MIMAT0007886 |
| hsa-miR-10a | UACCCUGUAGAUCCGAAUUUGUG | 583 | MIMAT0000253 |
| hsa-miR-184 | UGGACGGAGAACUGAUAAGGGU | 584 | MIMAT0000454 |
| hsa-miR-576-5p | AUUCUAAUUUCUCCACGUCUUU | 585 | MIMAT0003241 |
| hsa-miR-421 | AUCAACAGACAUUAAUUGGGCGC | 586 | MIMAT0003339 |
| hsa-miR-373 | GAAGUGCUUCGAUUUUGGGGUGU | 587 | MIMAT0000726 |
| hsa-miR-2053 | GUGUUAAUUAAACCUCUAUUUAC | 588 | MIMAT0009978 |
| hsa-miR-22 | AAGCUGCCAGUUGAAGAACUGU | 589 | MIMAT0000077 |
| hsa-miR-30c | UGUAAACAUCCUACACUCUCAGC | 590 | MIMAT0000244 |
| hsa-miR-374b | AUAUAAUACAACCUGCUAAGUG | 591 | MIMAT0004955 |
| hsa-miR-103-2* | AGCUUCUUUACAGUGCUGCCUUG | 592 | MIMAT0009196 |
| hsa-miR-10b | UACCCUGUAGAACCGAAUUUGUG | 593 | MIMAT0000254 |
| hsa-miR-519a | AAAGUGCAUCCUUUUAGAGUGU | 594 | MIMAT0002869 |
| hsa-miR-553 | AAAACGGUGAGAUUUUGUUUU | 595 | MIMAT0003216 |
| hsa-miR-609 | AGGGUGUUUCUCUCAUCUCU | 596 | MIMAT0003277 |
| hsa-miR-628-5p | AUGCUGACAUAUUUACUAGAGG | 597 | MIMAT0004809 |
| hsa-miR-1538 | CGGCCCGGGCUGCUGCUGUUCCU | 598 | MIMAT0007400 |
| hsa-miR-206 | UGGAAUGUAAGGAAGUGUGUGG | 599 | MIMAT0000462 |
| hsa-miR-19a | UGUGCAAAUCUAUGCAAAACUGA | 600 | MIMAT0000073 |
| hsa-miR-362-5p | AAUCCUUGGAACCUAGGUGUGAGU | 601 | MIMAT0000705 |
| hsa-miR-196b* | UCGACAGCACGACACUGCCUUC | 602 | MIMAT0009201 |
| hsa-miR-9* | AUAAAGCUAGAUAACCGAAAGU | 603 | MIMAT0000442 |
| hsa-miR-220b | CCACCACCGUGUCUGACACUU | 604 | MIMAT0004908 |
| hsa-miR-365 | UAAUGCCCCUAAAAAUCCUUAU | 605 | MIMAT0000710 |
| hsa-miR-1471 | GCCCGCGUGUGGAGCCAGGUGU | 606 | MIMAT0007349 |
| hsa-miR-1179 | AAGCAUUCUUUCAUUGGUUGG | 607 | MIMAT0005824 |
| hsa-miR-624* | UAGUACCAGUACCUUGUGUUCA | 608 | MIMAT0003293 |
| hsa-miR-128 | UCACAGUGAACCGGUCUCUUU | 609 | MIMAT0000424 |
| hsa-miR-579 | UUCAUUUGGUAUAAACCGCGAUU | 610 | MIMAT0003244 |
| hsa-miR-518d-3p | CAAAGCGCUUCCCUUUGGAGC | 611 | MIMAT0002864 |
| hsa-miR-224* | AAAAUGGUGCCCUAGUGACUACA | 612 | MIMAT0009198 |
| hsa-miR-551b* | GAAAUCAAGCGUGGGUGAGACC | 613 | MIMAT0004794 |
| hsa-miR-449b* | CAGCCACAACUACCCUGCCACU | 614 | MIMAT0009203 |
| hsa-miR-33a | GUGCAUUGUAGUUGCAUUGCA | 615 | MIMAT0000091 |
| hsa-miR-10a* | CAAAUUCGUAUCUAGGGGAAUA | 616 | MIMAT0004555 |
| hsa-miR-890 | UACUUGGAAAGGCAUCAGUUG | 617 | MIMAT0004912 |
| hsa-miR-802 | CAGUAACAAAGAUUCAUCCUUGU | 618 | MIMAT0004185 |
| hsa-miR-208b | AUAAGACGAACAAAAGGUUUGU | 619 | MIMAT0004960 |
| hsa-miR-620 | AUGGAGAUAGAUAUAGAAAU | 620 | MIMAT0003289 |
| hsa-miR-550 | AGUGCCUGAGGGAGUAAGAGCCC | 621 | MIMAT0004800 |
| hsa-miR-628-3p | UCUAGUAAGAGUGGCAGUCGA | 622 | MIMAT0003297 |
| hsa-miR-98 | UGAGGUAGUAAGUUGUAUUGUU | 623 | MIMAT0000096 |
| hsa-miR-224 | CAAGUCACUAGUGGUUCCGUU | 624 | MIMAT0000281 |
| hsa-miR-30c-2* | CUGGGAGAAGGCUGUUUACUCU | 625 | MIMAT0004550 |
| hsa-miR-448 | UUGCAUAUGUAGGAUGUCCCAU | 626 | MIMAT0001532 |
| hsa-miR-1914* | GGAGGGGUCCCGCACUGGGAGG | 627 | MIMAT0007890 |
| hsa-miR-514 | AUUGACACUUCUGUGAGUAGA | 628 | MIMAT0002883 |
| hsa-miR-544 | AUUCUGCAUUUUUAGCAAGUUC | 629 | MIMAT0003164 |
| hsa-miR-625* | GACUAUAGAACUUUCCCCCUCA | 630 | MIMAT0004808 |
| hsa-miR-501-5p | AAUCCUUUGUCCCUGGGUGAGA | 631 | MIMAT0002872 |
| hsa-miR-607 | GUUCAAAUCCAGAUCUAUAAC | 632 | MIMAT0003275 |
| hsa-miR-200b | UAAUACUGCCUGGUAAUGAUGA | 633 | MIMAT0000318 |
| hsa-miR-515-3p | GAGUGCCUUCUUUUGGAGCGUU | 634 | MIMAT0002827 |
| hsa-miR-183 | UAUGGCACUGGUAGAAUUCACU | 635 | MIMAT0000261 |
| hsa-miR-297 | AUGUAUGUGUGCAUGUGCAUG | 636 | MIMAT0004450 |
| hsa-miR-365* | AGGGACUUUCAGGGGCAGCUGU | 637 | MIMAT0009199 |
| hsa-miR-137 | UUAUUGCUUAAGAAUACGCGUAG | 638 | MIMAT0000429 |
| hsa-miR-588 | UUGGCCACAAUGGGUUAGAAC | 639 | MIMAT0003255 |
| hsa-miR-661 | UGCCUGGGUCUCUGGCCUGCGCG | 640 | MIMAT0003324 |
| | U | | |
| hsa-miR-130a | CAGUGCAAUGUUAAAAGGGCAU | 641 | MIMAT0000425 |
| hsa-miR-340 | UUAUAAAGCAAUGAGACUGAUU | 642 | MIMAT0004692 |
| hsa-miR-150 | UCUCCCAACCCUUGUACCAGUG | 643 | MIMAT0000451 |
| hsa-miR-1974 | UGGUUGUAGUCCGUGCGAGAAUA | 644 | MIMAT0009449 |
| hsa-miR-744 | UGCGGGGCUAGGGCUAACAGCA | 645 | MIMAT0004945 |
| hsa-miR-1979 | CUCCCACUGCUUCACUUGACUA | 646 | MIMAT0009454 |
| hsa-miR-193a-5p | UGGGUCUUUGCGGGCGAGAUGA | 647 | MIMAT0004614 |
| hsa-miR-577 | UAGAUAAAAUAUUGGUACCUG | 648 | MIMAT0003242 |
| hsa-miR-190b | UGAUAUGUUUGAUAUUGGGUU | 649 | MIMAT0004929 |
| hsa-miR-30b* | CUGGGAGGUGGAUGUUUACUUC | 650 | MIMAT0004589 |
| hsa-miR-653 | GUGUUGAAACAAUCUCUACUG | 651 | MIMAT0003328 |
| hsa-miR-144* | GGAUAUCAUCAUAUACUGUAAG | 652 | MIMAT0004600 |
| hsa-miR-518f* | CUCUAGAGGGAAGCACUUUCUC | 653 | MIMAT0002841 |
| hsa-miR-1914 | CCCUGUGCCCGGCCCACUUCUG | 654 | MIMAT0007889 |
| hsa-miR-1913 | UCUGCCCCCUCCGCUGCUGCCA | 655 | MIMAT0007888 |
| hsa-miR-219-2-3p | AGAAUUGUGGCUGGACAUCUGU | 656 | MIMAT0004675 |
| hsa-miR-539 | GGAGAAAUUAUCCUUGGUGUGU | 657 | MIMAT0003163 |
| hsa-miR-26a-2* | CCUAUUCUUGAUUACUUGUUUC | 658 | MIMAT0004681 |
| hsa-miR-888 | UACUCAAAAAGCUGUCAGUCA | 659 | MIMAT0004916 |
| hsa-miR-545 | UCAGCAAACAUUUAUUGUGUGC | 660 | MIMAT0003165 |
| hsa-miR-29b | UAGCACCAUUUGAAAUCAGUGUU | 661 | MIMAT0000100 |
| hsa-miR-208a | AUAAGACGAGCAAAAAGCUUGU | 662 | MIMAT0000241 |
| hsa-miR-708* | CAACUAGACUGUGAGCUUCUAG | 663 | MIMAT0004927 |
| hsa-miR-1539 | UCCUGCGCGUCCCAGAUGCCC | 664 | MIMAT0007401 |
| hsa-miR-181c | AACAUUCAACCUGUCGGUGAGU | 665 | MIMAT0000258 |
| hsa-miR-520d-5p | CUACAAAGGGAAGCCCUUUC | 666 | MIMAT0002855 |
| hsa-miR-1254 | AGCCUGGAAGCUGGAGCCUGCAGU | 667 | MIMAT0005905 |
| hsa-miR-2113 | AUUUGUGCUUGGCUCUGUCAC | 668 | MIMAT0009206 |
| hsa-miR-301a | CAGUGCAAUAGUAUUGUCAAAGC | 669 | MIMAT0000688 |
| hsa-miR-146a | UGAGAACUGAAUUCCAUGGGUU | 670 | MIMAT0000449 |
| hsa-miR-548d-5p | AAAAGUAAUUGUGGUUUUUGCC | 671 | MIMAT0004812 |
| hsa-miR-381 | UAUACAAGGGCAAGCUCUCUGU | 672 | MIMAT0000736 |
| hsa-miR-218-1* | AUGGUUCCGUCAAGCACCAUGG | 673 | MIMAT0004565 |
| hsa-miR-1912 | UACCCAGAGCAUGCAGUGUGAA | 674 | MIMAT0007887 |
| hsa-miR-1207-5p | UGGCAGGGAGGCUGGGAGGGG | 675 | MIMAT0005871 |
| hsa-miR-570 | CGAAAACAGCAAUUACCUUUGC | 676 | MIMAT0003235 |
| hsa-miR-491-5p | AGUGGGGAACCCUUCCAUGAGG | 677 | MIMAT0002807 |
| hsa-miR-572 | GUCCGCUCGGCGGUGGCCCA | 678 | MIMAT0003237 |
| hsa-miR-548c-3p | CAAAAAUCUCAAUUACUUUUGC | 679 | MIMAT0003285 |
| hsa-miR-29a | UAGCACCAUCUGAAAUCGGUUA | 680 | MIMAT0000086 |
| hsa-miR-302a* | ACUUAAACGUGGAUGUACUUGCU | 681 | MIMAT0000683 |
| hsa-miR-1909 | CGCAGGGGCCGGGUGCUCACCG | 682 | MIMAT0007883 |
| hsa-miR-1252 | AGAAGGAAAUUGAAUUCAUUUA | 683 | MIMAT0005944 |
| hsa-miR-299-3p | UAUGUGGGAUGGUAAACCGCUU | 684 | MIMAT0000687 |
| hsa-miR-373* | ACUCAAAAUGGGGGCGCUUUCC | 685 | MIMAT0000725 |
| hsa-miR-362-3p | AACACACCUAUUCAAGGAUUCA | 686 | MIMAT0004683 |
| hsa-miR-521 | AACGCACUUCCCUUUAGAGUGU | 687 | MIMAT0002854 |
| hsa-miR-200a | UAACACUGUCUGGUAACGAUGU | 688 | MIMAT0000682 |
| hsa-miR-1972 | UCAGGCCAGGCACAGUGGCUCA | 689 | MIMAT0009447 |
| hsa-miR-665 | ACCAGGAGGCUGAGGCCCCU | 690 | MIMAT0004952 |
| hsa-miR-548m | CAAAGGUAUUUGUGGUUUUUG | 691 | MIMAT0005917 |
| hsa-miR-626 | AGCUGUCUGAAAAUGUCUU | 692 | MIMAT0003295 |
| hsa-miR-384 | AUUCCUAGAAAUUGUUCAUA | 693 | MIMAT0001075 |
| hsa-miR-30e | UGUAAACAUCCUUGACUGGAAG | 694 | MIMAT0000692 |
| hsa-miR-93 | CAAAGUGCUGUUCGUGCAGGUAG | 695 | MIMAT0000093 |
| hsa-miR-383 | AGAUCAGAAGGUGAUUGUGGCU | 696 | MIMAT0000738 |
| hsa-miR-1537 | AAAACCGUCUAGUUACAGUUGU | 697 | MIMAT0007399 |
| hsa-miR-548l | AAAAGUAUUUGCGGGUUUUGUC | 698 | MIMAT0005889 |
| hsa-miR-338-3p | UCCAGCAUCAGUGAUUUUGUUG | 699 | MIMAT0000763 |
| hsa-miR-642 | GUCCCUCUCCAAAUGUGUCUUG | 700 | MIMAT0003312 |
| hsa-miR-30c-1* | CUGGGAGAGGGUUGUUUACUCC | 701 | MIMAT0004674 |
| hsa-miR-142-5p | CAUAAAGUAGAAAGCACUACU | 702 | MIMAT0000433 |
| hsa-miR-7-1* | CAACAAAUCACAGUCUGCCAUA | 703 | MIMAT0004553 |
| hsa-miR-26a | UUCAAGUAAUCCAGGAUAGGCU | 704 | MIMAT0000082 |
| hsa-miR-664 | UAUUCAUUUAUCCCCAGCCUACA | 705 | MIMAT0005949 |
| hsa-miR-363 | AAUUGCACGGUAUCCAUCUGUA | 706 | MIMAT0000707 |
| hsa-miR-660 | UACCCAUUGCAUAUCGGAGUUG | 707 | MIMAT0003338 |
| hsa-miR-561 | CAAAGUUUAAGAUCCUUGAAGU | 708 | MIMAT0003225 |
| hsa-miR-29c | UAGCACCAUUUGAAAUCGGUUA | 709 | MIMAT0000681 |
| hsa-miR-202* | UUCCUAUGCAUAUACUUCUUUG | 710 | MIMAT0002810 |
| hsa-miR-432* | CUGGAUGGCUCCUCCAUGUCU | 711 | MIMAT0002815 |
| hsa-miR-675* | CUGUAUGCCCUCACCGCUCA | 712 | MIMAT0006790 |
| hsa-miR-377 | AUCACACAAAGGCAACUUUUGU | 713 | MIMAT0000730 |
| hsa-miR-451 | AAACCGUUACCAUUACUGAGUU | 714 | MIMAT0001631 |
| hsa-miR-148b* | AAGUUCUGUUAUACACUCAGGC | 715 | MIMAT0004699 |
| hsa-miR-424 | CAGCAGCAAUUCAUGUUUUGAA | 716 | MIMAT0001341 |
| hsa-miR-431 | UGUCUUGCAGGCCGUCAUGCA | 717 | MIMAT0001625 |
| hsa-miR-1247 | ACCCGUCCCGUUCGUCCCCGGA | 718 | MIMAT0005899 |
| hsa-miR-651 | UUUAGGAUAAGCUUGACUUUUG | 719 | MIMAT0003321 |
| hsa-miR-103-as | UCAUAGCCCUGUACAAUGCUGCU | 720 | MIMAT0007402 |

Alternatively, or in addition to, the reagent can be for quantitation of at least 1, at least 2, at least 3, at least 4, at least 5, at least 6, at least 7, at least 8, at least 9 or at least 10 protein biomarkers selected from TABLE 2

TABLE 2

| | Protein | Gene |
|---|---|---|
| 1 | a2-Macroglobulin | A2M |
| 2 | a-Actinin-1 | ACTN1 |
| 3 | ABC Transporter | ABCG1 |
| 4 | Adiponectin | PPARG, NR1C3 |
| 5 | Adrenomedullin | ADM |
| 6 | CD166 Antigen | ALCAM |
| 7 | ANG-2, angiopoietin-2 | TEK, TIE2 |
| 8 | Annexin-2 | ANXA2, ANX2 |
| 9 | natriuretic peptide precursor A | ANP |
| 10 | apolipoprotein A1 | APOA1 |
| 11 | apolipoprotein A2 | APOA2 |
| 12 | apolipoprotein B | APOB |
| 13 | apolipoprotein C1 | APOC1 |
| 14 | apolipoprotein C3 | APOC3 |
| 15 | apolipoprotein E | APOE |
| 16 | apolipoprotein H (beta-2-glycoprotein I) | APOH |
| 17 | Clusterin, ApoJ | CLU |
| 18 | Antithrombin III | SERPINC1, AT3 |
| 19 | B cell attracting chemokine 1 | CXCL13, BCA-1 |
| 20 | Nerve Growth Factor, beta polypeptide | NGFB |
| 21 | Complement protein C1Q | C1QA |
| 22 | Caspase 4 | CASP1 |
| 23 | CCL1 | CCL1 |
| 24 | CCL14 | CCL14 |
| 25 | CCL15 | CCL15 |
| 26 | CCL18 | CCL18 |
| 27 | CCL21 | CCL21 |
| 28 | CCL28 | CCL28 |
| 29 | CCL9 | CCL9 |
| 30 | CD40 Ligand | CD40LG |
| 31 | CD44 | CD44 |
| 32 | CD52 | CD52 |
| 33 | CD53 | CD53 |
| 34 | cytokine receptor-like factor 1 | CRLF1 |
| 35 | CRP | CRP |
| 36 | colony stimulating factor 2 receptor, alpha, low-affinity | CSF2RA |
| | (granulocyte-macrophage) | |
| 37 | CTACK | CCL27 |
| 38 | CXCL11 | CXCL11 |
| 39 | CXCL14 | CXCL14 |
| 40 | CXCL16 | CXCL16 |
| 41 | Cystatin C | CST3 |
| 42 | D-dimer, fibrin degradation product | FGG, FGA, FGB |
| 43 | Epidermal growth factor | EGF |
| 44 | Endothelin-1 | EDN1 |
| 45 | En-RAGE, S100 calcium binding protein A12 | S100A12 |
| 46 | Eotaxin | CCL11 |
| 47 | E-Selectin, endothelial adhesion molecule 1 | SELE |
| 48 | fatty acid binding protein 3 | FABP3 |
| 49 | Factor II, thrombin | F2 |
| 50 | Factor V | F5 |
| 51 | Factor VII | F7 |
| 52 | Factor VIII | F8 |
| 53 | Fas, TNF receptor superfamily, member 6 | FAS |
| 54 | Fas-Ligand, TNF superfamily, member 6 | FASLG |
| 55 | Fc fragment of IgE | FCER1G |
| 56 | Fetuin A, alpha-2-HS-glycoprotein | AHSG |
| 57 | FGF-basic, fibroblast growth factor 2 (basic) | FGF2 |
| 58 | Fibrinogen | FGG, FGA, FGB |
| 59 | fibronectin 1 | FN1 |
| 60 | Fractalkine | CX3CL1 |
| 61 | frizzled-related protein | FRZB |
| 62 | Galectin-3 | LGALS3 |
| 63 | colony stimulating factor 3 (granulocyte) | CSF3 |
| 64 | growth differentiation factor 15 | GDF-15 |
| 65 | Granulin | GRN |
| 66 | GROa | CXCL1 |
| 67 | Haptoglobin | HP |
| 68 | fatty acid binding protein 3 | FABP3 |
| 69 | hepatocyte growth factor | HGF |
| 70 | Hsp-27, heat shock 27 kDa protein 1 | HSPB1 |
| 71 | integrin-binding sialoprotein | IBSP |
| 72 | ICAM-1, intercellular adhesion molecule 1 (CD54) | ICAM1 |
| 73 | interferon, alpha 2 | IFNA2 |
| 74 | interferon, gamma | IFNG |
| 75 | interferon gamma receptor 1 | IFNGR1 |
| 76 | IGF-1, insulin-like growth factor 1 (somatomedin C) | IGF1 |
| 77 | insulin-like growth factor binding protein 1 | IGFBP1 |
| 78 | insulin-like growth factor binding protein 3 | IGFBP3 |
| 79 | insulin-like growth factor binding protein 4 | IGFBP4 |
| 80 | insulin-like growth factor binding protein 6 | IGFBP6 |
| 81 | interleukin 10 | IL10 |
| 82 | Interleukin 12b, IL-12(p40) | IL12B |
| 83 | interleukin 16 | IL16 |
| 84 | interleukin 18 | IL18 |
| 85 | interleukin 1 alpha | IL1A |
| 86 | Interleukin 1 beta | IL1B |
| 87 | Interleukin 1 receptor-like 4 | IL1RL1 |
| 88 | Interleukin 2 receptor alpha | IL2RA |
| 89 | interleukin 3 | IL3 |
| 90 | interleukin 5 | IL5 |
| 91 | interleukin 6 | IL6 |
| 92 | interleukin 7 | IL7 |
| 93 | interleukin 8 | IL8 |
| 94 | IP-10 | CXCL10 |
| 95 | I-TAC | CXCL11 |
| 96 | lymphocyte cytosolic protein 1 | LCP1 |
| 97 | low density lipoprotein receptor | LDLR |
| 98 | Leptin | LEP |
| 99 | lectin, galactoside-binding, soluble, 3 binding protein | LGALS3BP |
| 100 | leukemia inhibitory factor | LIF |
| 101 | oxidised low density lipoprotein (lectin-like) receptor 1 | OLR1 |
| 102 | lipoprotein, Lp(a) | LPA |
| 103 | LpPLA2, lipopreotein-associated phospholipase A2 | PLA2G7 |
| 104 | L-Selectin, lymphocyte adhesion molecule 1 | SELL |
| 105 | Lysozyme | LYZ |
| 106 | MCP-1 | CCL2 |
| 107 | MCP-2 | CCL8 |
| 108 | MCP-3 | CCL7 |
| 109 | MCP-4 | CCL13 |
| 110 | MCP-5 | CCL12 |
| 111 | M-CSF, colony stimulating factor 1 (macrophage) | CSF1 |
| 112 | MDC, CCL22 | CCL22 |
| 113 | matrix Gla protein | MGP |
| 114 | macrophage migration inhibitory factor | MIF |
| 115 | MIG | CXCL9 |
| 116 | MIP-1a, Macrophage inflammatory protein 1-alpha | CCL3 |
| 117 | MIP-1 alpha P | CCL3L1 |
| 118 | MIP-1b | CXCL4 |
| 119 | MIP-2a, GROb | CXCL2 |
| 120 | MIP-2b, GROg | CXCL3 |
| 121 | MIP-3B, Macrophage inflammatory protein 3 beta | CCL19 |
| 122 | MMP-10, matrix metalloproteinase 10 | MMP10 |
| 123 | MMP-2, matrix metallopeptidase 2 | MMP2 |
| 124 | MMP-9, matrix metallopeptidase 9 | MMP9 |
| 125 | MPO, myeloperoxidase | MPO |
| 126 | myelin protein zero-like 1 | MPZL1 |
| 127 | major histocompatibility complex, class I-related | MR1 |
| 128 | NT-pro-BNP | NPPB |
| 129 | oncostatin M | OSM |
| 130 | Osteopontin | SPP1 |
| 131 | Osteoprotegerin, Tumor necrosis factor receptor superfamily | TNFRSF11B |
| | member 11B | |
| 132 | Ox-LDL receptor | OLR1 |
| 133 | PAI-1, plasminogen activator inhibitor type 1 | SERPINE1 |
| 134 | PAI-1 (total) | SERPINE1 |
| 135 | pregnancy-associated plasma protein A | PAPPA |
| 136 | proprotein convertase subtilisin/kexin type 9 | PCSK9 |
| 137 | platelet-derived growth factor beta | PDGFB |
| 138 | platelet derived growth factor C | PDGFC |
| 139 | platelet/endothelial cell adhesion molecule, CD31 antigen | PECAM1 |
| 140 | phospholipase A2, group VII | PLA2G7 |
| 141 | P-Selectin | SELP |
| 142 | prostaglandin D2 synthase | PTGDS |
| 143 | renal tumor antigen | RAGE |
| 144 | RANTES | CCL5 |
| 145 | Renin, Angiotensinogenase | REN |
| 146 | Resistin | RETN |
| 147 | Rho GDP dissociation inhibitor (GDI) beta | ARHGDIB |
| 148 | regulator of G-protein signalling 1 | RGS1 |
| 149 | regulator of G-protein signalling 10 | RGS10 |
| 150 | S100 calcium binding protein A8 | S100A8 |
| 151 | S100 calcium binding protein A9 | S100A9 |
| 152 | serum amyloid A1 | SAA |
| 153 | SAP, SH2 domain protein 1A | SH2D1A |
| 154 | SCF, KIT ligand | KITLG |
| 155 | SCGFb | CLEC11A |
| 156 | SDF-1 | CXCL12 |
| 157 | SDF-1a | CXCL12 |
| 158 | group IID secretory phospholipase A2 (sPLA2) | PLA2G2D |
| 159 | frizzled-related protein | FRZB |
| 160 | solute carrier family 11 | SLC11A1 |
| 161 | suppressor of cytokine signaling 3 | SOCS3 |
| 162 | Thrombomodulin | THBD |
| 163 | Thrombospondin R, CD36 molecule (thrombospondin receptor) | CD36 |
| 164 | Thrombospondin-1 | THBS1 |
| 165 | TIMP-1, metallopeptidase inhibitor 1 | TIMP1 |
| 166 | TIMP-2, metallopeptidase inhibitor 2 | TIMP2 |
| 167 | TIMP-3, metallopeptidase inhibitor 3 | TIMP3 |
| 168 | TIMP-4, metallopeptidase inhibitor 3 | TIMP4 |
| 169 | tenascin C | TNC |
| 170 | TNFa, tumor necrosis factor (TNF superfamily, member 2) | TNFA |
| 171 | tumor necrosis factor, alpha-induced protein 2 | TNFAIP2 |
| 172 | tumor necrosis factor, alpha-induced protein 6 | TNFAIP6 |
| 173 | TNFb, lymphotoxin alpha (TNF superfamily, member 1) | LTA |
| 174 | tumor necrosis factor receptor superfamily, member 1A, TNF-RI | TNFRSF1A |
| 175 | tumor necrosis factor receptor superfamily, member 1B, TNF- | TNFRSF1B |
| | RII | |
| 176 | tumor necrosis factor (ligand) superfamily, member 11, | TNFSF11 |
| | TRANCE, RANKL | |
| 177 | TRAIL, tumor necrosis factor (ligand) superfamily, member 10 | TNFSF10 |
| 178 | plasminogen activator, urokinase | PLAU |
| 179 | Vasopressin-neurophysin 2-copeptin | AVP |
| 180 | vascular cell adhesion molecule 1 | VCAM1 |
| 181 | vascular endothelial growth factor | VEGF |
| 182 | von Willebrand factor | VWF |
| 183 | WARS, tryptophanyl-tRNA synthetase | WARS |
| 184 | WNT1 inducible signaling pathway protein 1 | WISP1 |
| 185 | wingless-type MMTV integration site family, member 4 | WNT4 |

In certain embodiments, the protein biomarkers are selected from IL-16, sFas, Fas ligand, MCP-3, HGF, CTACK, EOTAXIN, adiponectin, IL-18, TIMP.4, TIMP.1, CRP, VEGF, and EGF.

The kits may further include a software package for statistical analysis of one or more phenotypes, and may include a reference database for calculating the probability of classification. The kit may include reagents employed in the various methods, such as devices for withdrawing and handling blood samples, second stage antibodies, ELISA reagents, tubes, spin columns, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the Internet to access the information at a removed site. Any convenient means may be present in the kits.

In an additional embodiment, the methods assays and kits disclosed herein can be used to detect a biomarker in a pooled sample. This method is particularly useful when only a small amount of multiple samples are available (for example, archived clinical sample sets) and/or to create useful datasets relevant to a disease or control population. In this regard, equal amounts (for example, about 10 μL, about 15 μL, about 20 μL, about 30 μL, about 40 μL, about 50 μL, or more) of a sample can be obtained from multiple (about 2, 5, 10, 15, 20, 30, 50, 100 or more) individuals. The individuals can be matched by various indicia. The indicia can include age, gender, history of disease, time to event, etc. The equal amounts of sample obtained from each individual can be pooled and analyzed for the presence of one or more biomarkers. The results can be used to create a reference set, make predictions, determine biomarkers associated with a given condition, etc by using the prediction and classifying models described herein. One of skill in the art will readily appreciate the many uses of this method and that it is in no way limited to the miRNAs, proteins, and disease states disclosed herein. In fact, this method can be used to detect DNA, RNA (mRNA, miRNA, hairpin precursor RNA, RNP), proteins, and the like, associated with a variety of diseases and conditions.

### DEFINITIONS

Terms used herein are defined as set forth below unless otherwise specified.

The term “monitoring” as used herein refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication. In particular, the term “monitoring” can refer to atherosclerosis staging, atherosclerosis prognosis, vascular inflammation levels, assessing extent of atherosclerosis progression, monitoring a therapeutic response, predicting a coronary calcium score, or distinguishing stable from unstable manifestations of atherosclerotic disease.

The term “quantitative data” as used herein refers to data associated with any dataset components (e.g., miRNA markers, protein markers, clinical indicia, metabolic measures, or genetic assays) that can be assigned a numerical value. Quantitative data can be a measure of the DNA, RNA, or protein level of a marker and expressed in units of measurement such as molar concentration, concentration by weight, etc. For example, if the marker is a protein, quantitative data for that marker can be protein expression levels measured using methods known to those of skill in the art and expressed in mM or mg/dL concentration units.

The term “mammal” as used herein includes both humans and non-humans and include but is not limited to humans, non-human primates, canines, felines, murines, bovines, equines, and porcines.

The term “pseudo coronary calcium score” as used herein refers to a coronary calcium score generated using the methods as disclosed herein rather than through measurement by an imaging modality. One of skill in the art would recognize that a pseudo coronary calcium score may be used interchangeably with a coronary calcium score generated through measurement by an imaging modality.

The term percent “identity” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

In certain embodiments, the “effectiveness” of a treatment regimen is determined. A treatment regimen is considered effective based on an improvement, amelioration, reduction of risk, or slowing of progression of a condition or disease. Such a determination is readily made by one of skill in the art.

### Example 1

### miRNA Analysis in Pooled Samples

The pooling approach utilized in this study accomplished two goals: a) to investigate the ability of the Exiqon Locked Nucleic Acid (LNA™) technology to identify miRNAs in serum and b) to utilize minimum volumes from precious archived clinical samples for testing.

In order to evaluate the ability of the LNA™ technology to identify miRNAs in serum, 52 pools were created using archived serum samples from a prospective study (Marshfield Clinical Personalized Medicine Research Project (PMRP), Personalized Medicine, 2(1): 49-79 (2005)). Twenty-six of the pools represented cases and 26 pools represented controls. Each pool contained equivalent volumes (50 μL) of serum sample from each of 5 individuals that were matched for age (selected from the eight 5-year ranges between 40 and 80 year old individuals), gender, and time to event for cases (i.e, MI within 0-6 mos, MI within 6-12 mos, etc). The matching for the later was approximate. Cases were subjects with an MI or hospitalized unstable angina within five years from blood draw. Controls were subjects that did not have either of these events within five years from blood draw. The sample was evaluated as a classification problem and the test performance was judged using the area under the curve (AUC).

The performance of the test in terms of AUC depends on the distribution of measured values (for individual markers) or of that of the score, which at the time of the experimental design was unknown. In order to estimate the expected performance of the test for a set of similar sample size with the actual experimental design (26 cases and 26 controls), a number of simulations were performed using different assumed distributions for the variables and number of samples in a pool. The assumed distributions used were: a) normal, b) chisq and c) log-normal. For each distribution and number of samples in a pool the appropriate number of “controls” was randomly selected and the corresponding number of cases was selected from a distribution with known shift in the mean, in order to represent differences between the populations. Therefore, for a pool of size M, select 26*M controls and 26*M cases were selected and each pooled sample is created by averaging the values of M samples. The process was repeated 500 times and a distribution of expected AUCs was estimated for a given number of pooled samples and population distance.

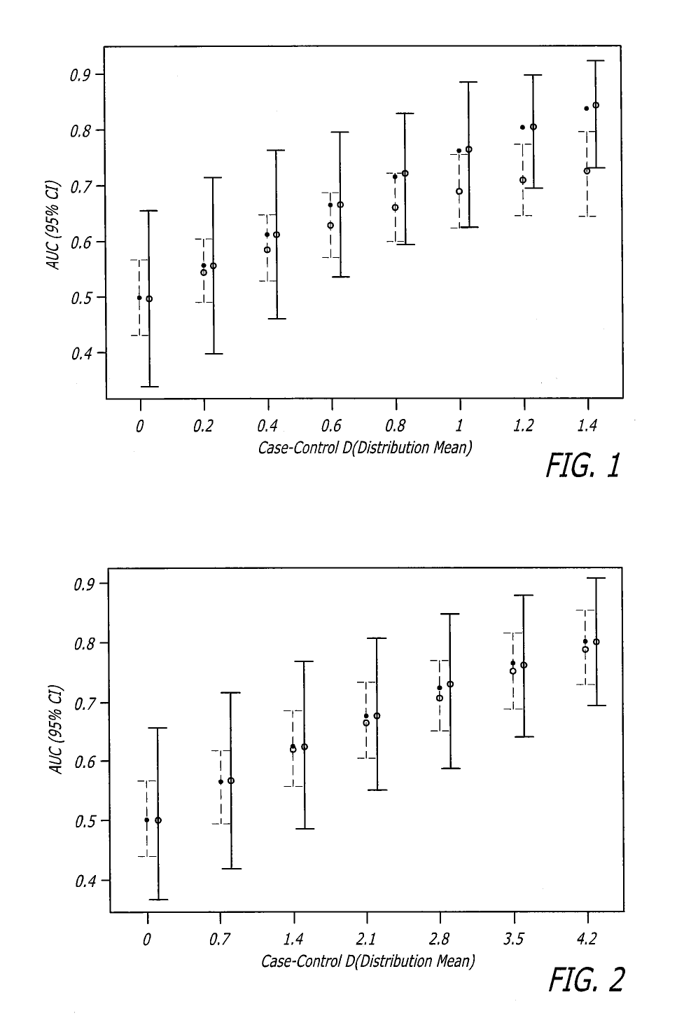

FIG. 1 shows the results for an assumed log-normal distribution of the biomarker concentration or score, using individual samples (open circles and solid error bars) and pooled samples (5 individual samples per pool) (open circles and dashed error bars). The solid black dots indicate the theoretical answer for individual measurements. One observes that the expected AUC consistently underestimates the true and expected AUC for individual samples, but the uncertainty range is smaller for the pooled samples. FIG. 2 displays the results for an assumed normal distribution of measurements. In this case, the pooled sample results are in excellent agreement with the theoretical and individual sample results. Again, the uncertainty of the pooled samples is smaller than the corresponding uncertainty of the human samples. An assumed chisq-distribution provided simulated results that were more in agreement with those obtained from the log-normal distribution. These simulations indicate that the results of pooled samples will provided a very good estimate of the expected AUC if the distribution of the human samples follows a normal distribution, otherwise the calculated AUC will be underestimated.

Thirty-eight miRNAs on 52 pooled samples were analyzed using EXIQON UniRT® LNA technology. Total RNA was extracted from the supplied serum samples (described above) using the QIAGEN RNEASY® Mini Kit Protocol (QIAGEN, Valenica, CA) with a slightly modified protocol.

Total RNA was extracted from serum using the QIAGEN RNEASY® Mini Kit. Serum was thawed on ice and centrifuge at 1000×g for 5 min in a 4° C. microcentrifuge. An aliquot of 200 μL of serum per sample was transferred to a new microcentrifuge tube and 750 ul of Qiazol mixture containing 0.94 μg/μL of MS2 bacteriophage was added to the serum. Tube was mixed and incubated for 5 min followed by the addition of 200 μL chloroform. Tube was mixed, incubated for 2 min and centrifuge at 12,000×g for 15 min in a 4° C. microcentrifuge. Upper aqueous phase was collected to a new microcentrifuge tube and 1.5 volume of 100% ethanol was added. Tube was mixed thoroughly and 750 μL of the sample was transferred to the QIAGEN RNEASY® Mini spin column in a collection tube followed by centrifugation at 15,000×g for 30 sec at room temperature. Process was repeated until remaining sample was loaded. The QIAGEN RNEASY® Mini spin column was rinsed with 700 μL QIAGEN RWT buffer and centrifuge at 15,000×g for 1 min at room temperature followed by another rinse with 500 μL QIAGEN RPE buffer and centrifuge at 15,000×g for 1 min at room temperature. Rinsing with 500 μL QIAGEN RPE buffer was repeated 2×. The QIAGEN RNEASY® Mini spin column was transferred to a new collection tube and centrifuge at 15,000×g for 2 min at room temperature. The QIAGEN RNEASY® Mini spin column was transferred to a new microcentrifuge tube and the lid was uncapped for 1 min to dry. RNA was eluted by adding 50 μL of RNase-free water to the membrane of the QIAGEN RNEASY® mini spin column and incubated for 1 min before centrifugation at 15,000×g for 1 min at room temperature. RNA was stored in −70° C. freezer until shipment on dry ice. Thirty-eight miRNAs were selected for analysis (Table 3).

TABLE 3

| | miRNA |
|---|---|
| 1 | hsa-let-7a |
| 2 | hsa-let-7b |
| 3 | hsa-let-7d |
| 4 | hsa-mir-1 |
| 5 | hsa-mir-106b |
| 6 | hsa-mir-10b |
| 7 | hsa-mir-125b |
| 8 | hsa-mir-126 |
| 9 | hsa-mir-146b-5p |
| 10 | hsa-mir-148a |
| 11 | hsa-mir-155 |
| 12 | hsa-mir-15a |
| 13 | hsa-mir-16 |
| 14 | hsa-mir-17 |
| 15 | hsa-mir-182 |
| 16 | hsa-mir-18a |
| 17 | hsa-mir-192 |
| 18 | hsa-mir-200c |
| 19 | hsa-mir-205 |
| 20 | hsa-mir-20a |
| 21 | hsa-mir-20b |
| 22 | hsa-mir-21 |
| 23 | hsa-mir-212 |
| 24 | hsa-mir-218 |
| 25 | hsa-mir-221 |
| 26 | hsa-mir-222 |
| 27 | hsa-mir-23a |
| 28 | hsa-mir-23b |
| 29 | hsa-mir-24 |
| 30 | hsa-mir-26a |
| 31 | hsa-mir-27a |
| 32 | hsa-mir-32 |
| 33 | hsa-mir-342-5p |
| 34 | hsa-mir-429 |
| 35 | hsa-mir-451 |
| 36 | hsa-mir-9 |
| 37 | hsa-mir-103 |
| 38 | hsa-mir-93 |

Each RNA sample was reverse transcribed (RT) into cDNA in three independent RT reactions and run as singlicate real-time PCR or qPCR reaction.

Each 384 well plate contained reactions for all the samples for 2 miRNA assays. Negative controls were included in the experiment: No template control (RNA replaced with water) in RT step, and a No enzyme control in the RT step (pooled RNA as template). All assays passed this quality control step in that the no template control and no enzyme control were negative.

An additional step in the real-time PCR analysis was performed to evaluate the specificity of the assays by generating a melting curve for each reaction. The appearance of a single peak during melting curve analysis is an indication that a single specific product was amplified during the qPCR process. The appearance of multiple melting curve peaks correspondingly provides an indication of multiple qPCR amplification products and is evidence of a lack of specificity. Any assays that showed multiple peaks have been excluded from the data set. The amplification curves were analyzed using the LIGHTCYCLER® software (Roche, Indianapolis, Ind.) both for determination of Cp (crossing point, i.e., the point where the measured signal crosses above a predesignated threshold value, indicating a measurable concentration of the target sequence) (by 2^{nd} derivative method) and for melting curve analysis.

PCR efficiency was also assessed by analysis of the PCR amplification curve with the LINREG® software (Open Source Software) The performance of five housekeeping miRNAs (miR-16, miR-93, miR-103, miR-192 & miR-451) was used to evaluate the quality of the RNA extracted from the supplied serum samples.

Twenty-four of the 38 miRNA targets were detected in the samples. Fifty of the samples (26 cases and 24 controls) were used to evaluate the expected performance of a classification analysis on these samples and to select miRNAs that predict status. The following methodologies were employed for building a model: a) a logistic regression approach and b) a penalized logistic regression approach using (L1 penalty-lasso). The selection of the terms that provided the best classification in a model was completed by a) conducting forward selection using the Bayesian Information criterion for the unpenalized logistic regression approach and b) a cross-validation based selection of the optimum penalty for the penalized approach. In the latter, since the penalty parameter drives the coefficients of the available parameters to zero, the resulting model contains only a reduced number of predictive miRNAs. In order to evaluate an objective measure of the performance, AUC was calculated using a prevalidated score. The prevalidation is very similar to a cross-validation approach, where the association of a “score” with a given outcome is based on values that for a given subject have been predicted from a model that was fit without using the specific subject in the training set. For this analysis prevalidated scores were calculated based on two approaches: a) k-fold cross-validation and b) leave-one-out cross validation. The prevalidation iteration has been repeated N times (where N is usually equal to 100-1000). The complete sequence of the analysis is as follows:

1) Fit a model on a subset of the data using logistic regression with BIC for model selection, or penalized logistic regression estimating the penalty function through a nested cross-validation in the training set;

2) For a k-fold cross-validation, the model is fitted on k−1 groups of samples;

3) For a leave-one-out cross-validation, the model is fitted in the M−1 samples where here M=50;

4) Using the fitted model, predict the score for the left-out samples (group k for the cross-validation and the single left-out sample for the leave-one-out cross-validation);

5) Once all the scores have been predicted for all the samples, calculate the AUC for the classification problem;

6) Repeat steps 1-3 N times to evaluate the variability of the AUC.

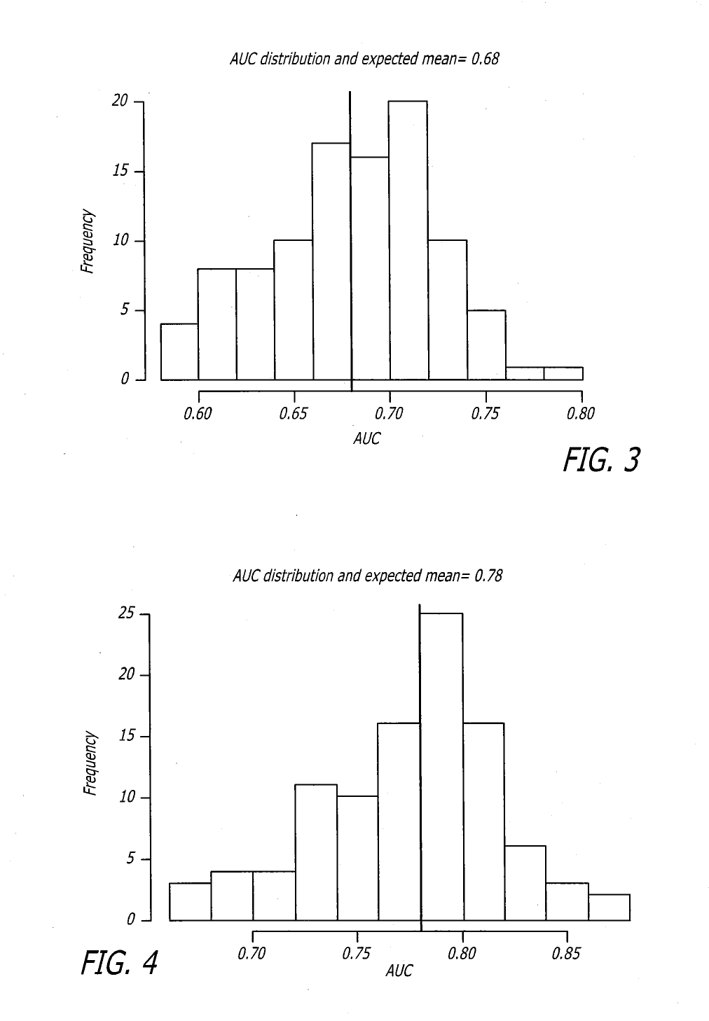

FIG. 3 presents the distribution of AUC values obtained using a penalized logistic regression model (L1 penalty-lasso) with 100 repeats of the prevalidation score calculation. Table 4 presents the top miRNAs selected during the process of model selection and fitting using penalized logistic regression (L1 penalty-lasso), and 10-fold cross-validation for prevalidated score calculation. The maximum number of times that a marker can be selected in this run is 1000 (100 repeats of score prevalidation ×10-fold cross validation during each repeat).

TABLE 4

| miR | Counts |
|---|---|
| miR.16 | 999 |
| miR.26a | 998 |
| miR.130a | 981 |
| miR.150 | 917 |
| miR.222 | 856 |
| miR.106b | 836 |
| miR.93 | 801 |
| miR.10b | 771 |
| miR.30c | 722 |
| miR.192 | 717 |
| let.7b | 579 |
| miR.20a | 436 |
| miR.107 | 313 |
| miR.20b | 239 |
| hsa.let.7f | 225 |
| miR.186 | 208 |
| miR.92a | 157 |

Table 5 presents the count of biomarkers selected using the leave-one-out (LOOV) cross-validation in combination with an L1 penalized logistic regression approach. The two methods provide highly overlapping sets of biomarkers, selected at approximately the same order. The difference in the counts is due to the number of samples in the set. The corresponding AUC is 0.66.

TABLE 5

| miR | Counts |
|---|---|
| miR.26a | 51 |
| miR.16 | 51 |
| miR.130a | 51 |
| miR.150 | 51 |
| miR.106b | 50 |
| miR.93 | 50 |
| miR.222 | 48 |
| miR.192 | 47 |
| miR.30c | 47 |
| miR.10b | 40 |
| let.7b | 32 |
| miR.20a | 26 |
| miR.20b | 16 |
| miR.107 | 16 |
| hsa.let.7f | 15 |
| miR.186 | 14 |
| miR.92a | 12 |
| miR.19a | 3 |

### Example 2

### Evaluation of miRNA in Individual Samples

A follow-up experiment concentrated on evaluating the detection and performance of miRNAs in individual serum samples (26 cases and 26 controls) using the EXIQON LNA™ technology described in Example 1. A total of 90 miRNAs (see Table 6) were screened, which included the miRNAs screened in the pooled samples. Fourty-four of the 90 miRNA targets were detected in the individual serum samples. The 24 miRs detected in the pooled samples were also detected in the individual samples and 20 additional miRNAs were detected in the individual samples. Five miRNAs were used for data normalization and were removed from the analysis.

TABLE 6

| | | Samples | Samples |
|---|---|---|---|
| | miRNA | 1-52 | 53-104 |
| 1 | hsa-let- | Yes* | Yes** |
| | 7a | | |
| 2 | hsa-let- | Yes* | Yes** |
| | 7b | | |
| 3 | hsa-let- | Yes* | Yes** |
| | 7d | | |
| 4 | hsa-mir-1 | No* | No** |
| 5 | hsa-mir- | Yes* | Yes** |
| | 106b | | |
| 6 | hsa-mir- | Yes* | Yes** |
| | 10b | | |
| 7 | hsa-mir- | No* | No** |
| | 125b | | |
| 8 | hsa-mir- | Yes* | Yes** |
| | 126 | | |
| 9 | hsa-mir- | No* | No** |
| | 146b-5p | | |
| 10 | hsa-mir- | Yes* | Yes** |
| | 148a | | |
| 11 | hsa-mir- | No* | No** |
| | 155 | | |
| 12 | hsa-mir- | Yes* | Yes** |
| | 15a | | |
| 13 | hsa-mir- | Yes* | Yes** |
| | 16 | | |
| 14 | hsa-mir- | Yes* | Yes** |
| | 17 | | |
| 15 | hsa-mir- | No* | No** |
| | 182 | | |
| 16 | hsa-mir- | No* | No** |
| | 18a | | |
| 17 | hsa-mir- | Yes* | Yes** |
| | 192 | | |
| 18 | hsa-mir- | No* | No** |
| | 200c | | |
| 19 | hsa-mir- | No* | No** |
| | 205 | | |
| 20 | hsa-mir- | Yes* | Yes** |
| | 20a | | |
| 21 | hsa-mir- | Yes* | Yes** |
| | 20b | | |
| 22 | hsa-mir- | Yes* | Yes** |
| | 21 | | |
| 23 | hsa-mir- | No* | No** |
| | 212 | | |
| 24 | hsa-mir- | No* | No** |
| | 218 | | |
| 25 | hsa-mir- | Yes* | Yes** |
| | 221 | | |
| 26 | hsa-mir- | Yes* | Yes** |
| | 222 | | |
| 27 | hsa-mir- | Yes* | Yes** |
| | 23a | | |
| 28 | hsa-mir- | Yes* | Yes** |
| | 23b | | |
| 29 | hsa-mir- | Yes* | Yes** |
| | 24 | | |
| 30 | hsa-mir- | Yes* | Yes** |
| | 26a | | |
| 31 | hsa-mir- | Yes* | Yes** |
| | 27a | | |
| 32 | hsa-mir- | No* | No** |
| | 32 | | |
| 33 | hsa-mir- | No* | No** |
| | 342-5p | | |
| 34 | hsa-mir- | No* | No** |
| | 429 | | |
| 35 | hsa-mir- | Yes* | Yes** |
| | 451 | | |
| 36 | hsa-mir-9 | No* | No** |
| 37 | hsa-mir- | Yes* | Yes** |
| | 103 | | |
| 38 | hsa-mir- | Yes* | Yes** |
| | 93 | | |
| 39 | hsa-let- | Yes** | Yes** |
| | 7c | | |
| 40 | hsa-let- | Yes** | Yes** |
| | 7f | | |
| 41 | hsa-mir- | Yes** | Yes** |
| | 107 | | |
| 42 | hsa-mir- | No** | No** |
| | 125a-3p | | |
| 43 | hsa-mir- | Yes** | Yes** |
| | 125a-5p | | |
| 44 | hsa-mir- | No** | No** |
| | 129-3p | | |
| 45 | hsa-mir- | No** | No** |
| | 129-5p | | |
| 46 | hsa-mir- | Yes** | Yes** |
| | 130a | | |
| 47 | hsa-mir- | No** | No** |
| | 130b | | |
| 48 | hsa-mir- | No** | No** |
| | 132 | | |
| 49 | hsa-mir- | No** | No** |
| | 135a | | |
| 50 | hsa-mir- | No** | No** |
| | 136 | | |
| 51 | hsa-mir- | Yes** | Yes** |
| | 146a | | |
| 52 | hsa-mir- | No** | No** |
| | 146b-3p | | |
| 53 | hsa-mir- | Yes** | Yes** |
| | 150 | | |
| 54 | hsa-mir- | No** | No** |
| | 181a | | |
| 55 | hsa-mir- | Yes** | Yes** |
| | 186 | | |
| 56 | hsa-mir- | No** | No** |
| | 195 | | |
| 57 | hsa-mir- | No** | No** |
| | 196a | | |
| 58 | hsa-mir- | Yes** | Yes** |
| | 199a-3p | | |
| 59 | hsa-mir- | Yes** | Yes** |
| | 199a-5p | | |
| 60 | hsa-mir- | Yes** | Yes** |
| | 19a | | |
| 61 | hsa-mir- | Yes** | Yes** |
| | 19b | | |
| 62 | hsa-mir- | No** | No** |
| | 208a | | |
| 63 | hsa-mir- | No** | No** |
| | 208b | | |
| 64 | hsa-mir- | No** | No** |
| | 210 | | |
| 65 | hsa-mir- | No** | No** |
| | 211 | | |
| 66 | hsa-mir- | No** | No** |
| | 214 | | |
| 67 | hsa-mir- | No** | No** |
| | 215 | | |
| 68 | hsa-mir- | Yes** | Yes** |
| | 22 | | |
| 69 | hsa-mir- | No** | No** |
| | 27b | | |
| 70 | hsa-mir- | No** | No** |
| | 28-5p | | |
| 71 | hsa-mir- | No** | No** |
| | 296-3p | | |
| 72 | hsa-mir- | No** | No** |
| | 296-5p | | |
| 73 | hsa-mir- | No** | No** |
| | 299-3p | | |
| 74 | hsa-mir- | No** | No** |
| | 299-5p | | |
| 75 | hsa-mir- | No** | No** |
| | 302a | | |
| 76 | hsa-mir- | No** | No** |
| | 302b | | |
| 77 | hsa-mir- | No** | No** |
| | 302c | | |
| 78 | hsa-mir- | Yes** | Yes** |
| | 30a | | |
| 79 | hsa-mir- | Yes** | Yes** |
| | 30c | | |
| 80 | hsa-mir- | Yes** | Yes** |
| | 30e | | |
| 81 | hsa-mir- | No** | No** |
| | 325 | | |
| 82 | hsa-mir- | No** | No** |
| | 330-3p | | |
| 83 | hsa-mir- | No** | No** |
| | 330-5p | | |
| 84 | hsa-mir- | Yes** | Yes** |
| | 331-3p | | |
| 85 | hsa-mir- | No** | No** |
| | 331-5p | | |
| 86 | hsa-mir- | No** | No** |
| | 340 | | |
| 87 | hsa-mir- | Yes** | Yes** |
| | 342-3p | | |
| 88 | hsa-mir- | No** | No** |
| | 34b | | |
| 89 | hsa-mir- | Yes** | Yes** |
| | 378 | | |
| 90 | hsa-mir- | Yes** | Yes** |
| | 92a | | |

*Assessed as part of Example 1,
**Assessed as part of Example 2

The same methodology described in Example 1 was utilized for analysis of this data set. Using a penalized logistic regression with a leave-one-out cross validation produced an AUC equal to 0.778. The number of times individual miRNAs were selected in the models used in the prevalidated score calculation is shown in Table 7 (50 models total since there were 50 samples). The average model size was −8 terms (top 8 miRNAs are indicated by “*”). The expected value is higher than the corresponding value obtained for the pooled data.

TABLE 7

| MiR | Counts |
|---|---|
| miR.378* | 50 |
| miR.92a* | 50 |
| miR.26a* | 50 |
| miR.130a* | 48 |
| miR.222* | 41 |
| miR.15a* | 38 |
| miR.125a.5p* | 33 |
| let.7b* | 28 |
| miR.331.3p | 25 |
| miR.221 | 18 |
| miR.30e | 9 |
| miR.199a.3p | 1 |
| miR.22 | 1 |
| miR.199a.5p | 1 |
| miR.20a | 1 |
| let.7a | 1 |

Table 8 provides the miRNAs selected when an L1 penalized logistic regression approach with 4-fold cross validation was applied to 50 individual samples. Again, considerable overlap in the markers and order is observed between the two methods. FIG. 4 presents the distribution of AUC values obtained from this analysis.

TABLE 8

| miR | Counts |
|---|---|
| miR.378 | 400 |
| miR.92a | 396 |
| miR.26a | 366 |
| miR.130a | 233 |
| miR.125a.5p | 172 |
| miR.222 | 152 |
| miR.15a | 146 |

### Example 3

### Analysis of Protein Biomarkers

Models were developed that included protein only data (from the Marshfield cohort utilized in Examples 1 and 2). A total of 47 unique protein biomarkers (Table 9) were analyzed. Serum samples were collected and kept frozen at −80° C., then thawed immediately prior to use. Each sample was analyzed in duplicate using two distinct detection technologies: xMAP® technology from Luminex (Austin, Tex.) and the SECTOR® Imager with MULTI-SPOT® technology from Meso Scale Discovery (MSD, Gaithersburg, Md.).

TABLE 9
Protein Biomarker

Adiponectin
ANG-2
b-NGF
CRP
CTACK
EGF
Eotaxin
FASLigand
GROa
HGF
IFN-a2
IL-12p40
IL-16
IL-18
IL-1a
IL-2Ra
IL-3
IP-10
I-TAC
Leptin
LIF
MCP-1
MCP-2
MCP-3
MCP-4
M-CSF
MIF
MIG
MIP-1a
MPO
NTproBNP
PAI-1
RANTES
Resistin
SCD40L
SCF
SCGF-b
SDF-1a
sE-Selectin
sFas
sICAM-1
sP-Selectin
TIMP-1
TIMP-4
TNF-b
TRAIL
VEGF

The Luminex xMAP technology utilizes analyte-specific antibodies that are pre-coated onto color-coded microparticles. Microparticles, standards and samples are pipetted into wells and the immobilized antibodies bind the analytes of interest. After an appropriate incubation period, the particles are re-suspended in wash buffer multiple times to remove any unbound substances. A biotinylated antibody cocktail specific to the analytes of interest is added to each well. Following a second incubation period and a wash to remove any unbound biotinylated antibody, streptavidin-phycoerythrin conjugate (Streptavidin-PE), which binds to the biotinylated detection antibodies, is added to each well. A final wash removes unbound Streptavidin-PE and the microparticles are resuspended in buffer and read using the Luminex analyzer. The analyzer uses a flow cell to direct the microparticles through a multi-laser detection system. One laser is microparticle-specific and determines which analyte is being detected. The other laser determines the magnitude of the phycoerythrin-derived signal, which is in direct proportion to the amount of analyte bound. Curves are constructed using the signals generated by the standards and protein biomarker concentrations of the samples are read off each curve. Sensitivity (Limit of Detection, LOD) and precision (intra- and inter-assay % CV) of the 47 Luminex protein biomarker assays is shown in Table 10.

TABLE 10

| Protein | LOD | Avg Intra | Avg Inter |
|---|---|---|---|
| Biomarker | (pg/mL) | Assay % CV | Assay % CV |
| Adiponectin | 682 | 9% | 11% |
| ANG-2 | 18 | 4% | 7% |
| b-NGF | 1 | 7% | 13% |
| CRP | 525 | 7% | 9% |
| CTACK | 25 | 10% | 10% |
| EGF | 9 | 5% | 14% |
| Eotaxin | 1 | 15% | 16% |
| FASLigand | 1 | 9% | 12% |
| GROa | 31 | 3% | 6% |
| HGF | 28 | 4% | 11% |
| IFN-a2 | 13 | 2% | 9% |
| IL-12p40 | 144 | 5% | 9% |
| IL-16 | 15 | 4% | 8% |
| IL-18 | 3 | 5% | 6% |
| IL-1a | 1 | 5% | 19% |
| IL-2Ra | 13 | 4% | 10% |
| IL-3 | 31 | 4% | 4% |
| IP-10 | 0 | 5% | 11% |
| I-TAC | 2 | 10% | 17% |
| Leptin | 28 | 6% | 8% |
| LIF | 66 | 28% | 31% |
| MCP-1 | 6 | 3% | 8% |
| MCP-2 | 1 | 7% | 10% |
| MCP-3 | 19 | 6% | 12% |
| MCP-4 | 2 | 4% | 11% |
| M-CSF | 8 | 4% | 7% |
| MIF | 24 | 5% | 12% |
| MIG | 6 | 7% | 7% |
| MIP-1a | 54 | 7% | 13% |
| MPO | 156 | 7% | 12% |
| NTproBNP | 96 | 7% | 55% |
| PAI-1 | 9 | 5% | 6% |
| RANTES | 4 | 7% | 6% |
| Resistin | 9 | 5% | 8% |
| SCD40L | 115 | 4% | 11% |
| SCF | 9 | 4% | 7% |
| SCGF-b | 1017 | 4% | 9% |
| SDF-1a | 23 | 8% | 10% |
| sE-Selectin | 7 | 3% | 7% |
| sFas | 6 | 5% | 6% |
| sICAM-1 | 70 | 6% | 7% |
| sP-Selectin | 218 | 4% | 9% |
| TIMP-1 | 17 | 5% | 6% |
| TIMP-4 | 27 | 5% | 41% |
| TNF-b | 8 | 5% | 13% |
| TRAIL | 24 | 3% | 8% |
| VEGF | 5 | 7% | 9% |

Ten of the 45 unique protein biomarkers were analyzed with a 10-plex assay on the MSD platform (Table 11).

TABLE 11
Protein Biomarker

CTACK
HGF
IL-16
IL-18
MCP-3
M-CSF
MIF
MIG
NTproBNP
TRAIL

The MSD technology utilizes specialized 96-well microtiterplates constructed with a carbon surface on the bottom of each plate. Antibodies specific for each protein biomarker are spotted in spatial arrays on the bottom of each well of the microtiterplate. Standards and samples are pipetted into the wells of the precoated plates and the immobilized antibodies bind the analytes of interest. After an appropriate incubation period, the plates are washed multiple times to remove any unbound substances. A cocktail of analyte-specific secondary antibodies labeled with a SULFO-TAG™ is added to each well. Following a second incubation period, the plates are again washed multiple times to remove any unbound materials and a specialized Read Buffer is added to each well. The plates are then placed into the SECTOR® Imager where an electric current is applied to the carbon electrode on the bottom of the microtiterplate. The SULFO-TAG™ labels bound to the specific secondary antibodies at each spot emit light upon this electrochemical stimulation, which is detected using a sensitive CCD camera. Curves are constructed using the signals generated by the standards and protein biomarker concentrations of the samples are read off each curve. Sensitivity (Limit of Detection, LOD) and precision (intra- and inter-assay % CV) of the 10 MSD protein biomarker assays is shown in Table 12.

TABLE 12

| Protein | % Detected > | Avg Intra Assay | Avg Inter Assay |
|---|---|---|---|
| Biomarker | LOD (pg/mL) | % CV (FI) | % CV (Conc) |
| CTACK | 99% | 9% | 23% |
| HGF | 99% | 7% | 15% |
| IL-16 | 99% | 9% | 11% |
| IL-18 | 99% | 6% | 8% |
| MCP-3 | 69% | 6% | 11% |
| M-CSF | 99% | 13% | 34% |
| MIF | 99% | 5% | 9% |
| MIG | 99% | 8% | 14% |
| NTproBNP | 99% | 6% | 27% |
| TRAIL | 99% | 9% | 179% |

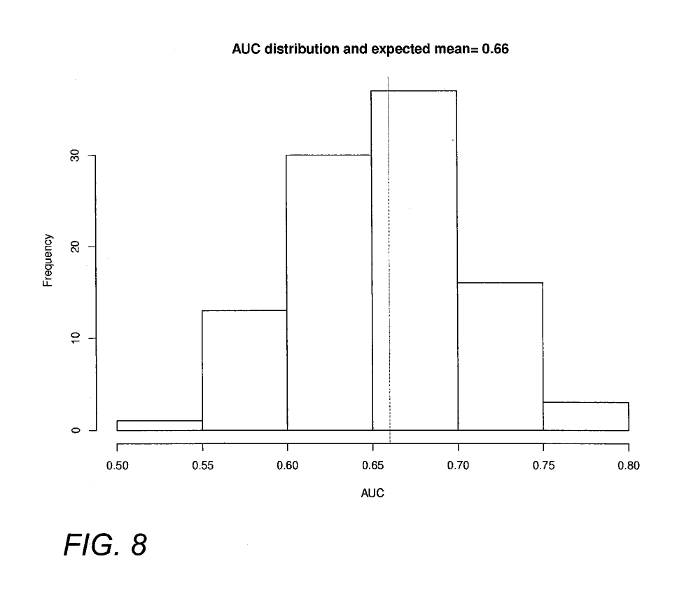

The models were built and performance was evaluated using the logistic regression approach with LOOV or k-fold cross-validation for the calculation of the prevalidated score as described above. FIG. 8 provides the distribution of the AUC values obtained from models based on proteins only using the k-fold cross-validation approach for predicting a prevalidated score. Table 13 provides the selection frequency of a protein marker in any of the cross-validated models. A higher count indicates that a marker has a consistent ability to classify cases from controls. The AUC using the LOOV approach for the calculation of a prevalidated score was calculated to be 0.698 and Table 14 provides the selection frequency of a marker within any of the models built using the LOOV methodology. The later AUC is within the uncertainty limits calculated from the k-fold cross-validation approach. Both methods select the same top markers.

TABLE 13

| Marker | Counts |
|---|---|
| sP-Selectin | 717 |
| MPO | 692 |
| Eotaxin | 536 |
| IL-16 | 361 |
| Resistin | 249 |
| VEGF | 205 |
| CRP | 204 |
| HGF | 113 |

TABLE 14

| Marker | Counts |
|---|---|
| sP-Selectin | 41 |
| MPO | 41 |
| Eotaxin | 38 |
| IL-16 | 38 |

### Example 4

### Combined Analysis of miRNA and Protein Biomarkers

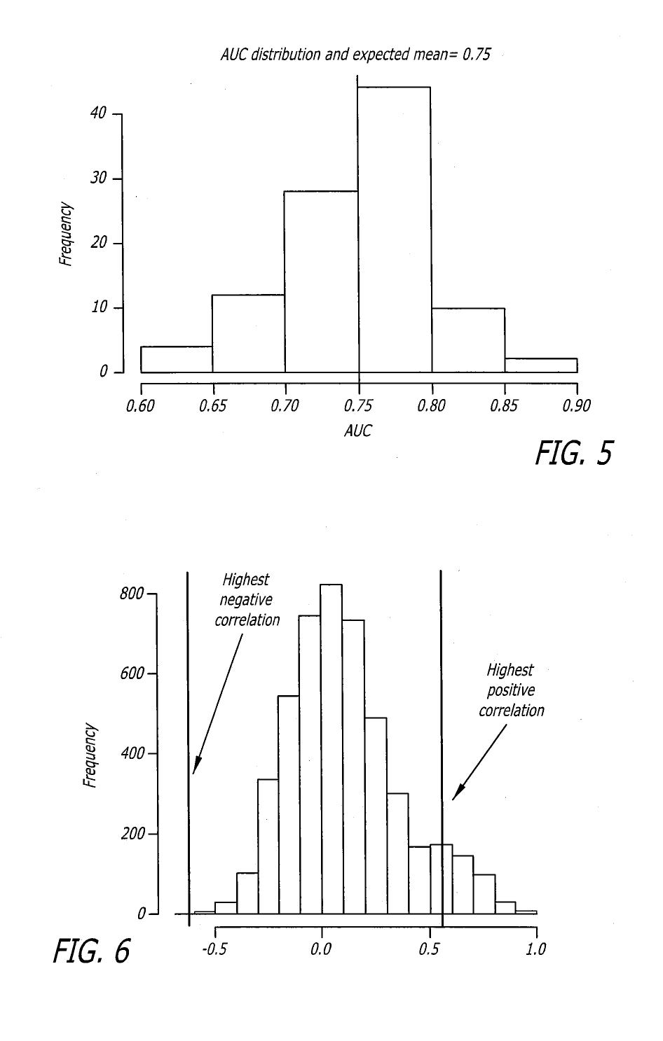

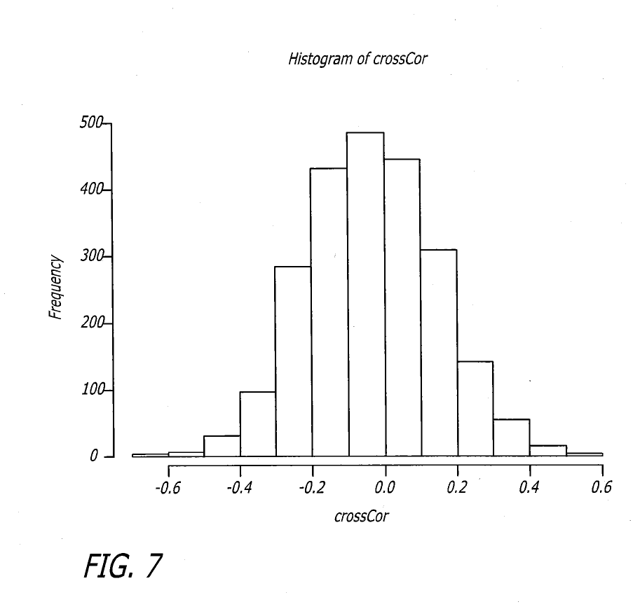

Models were developed that included both protein and miRNAs data (from Examples 1 and 2). The protein data across 47 biomarkers (from Example 3) were obtained using two distinct detection technologies: Luminex (Luminex Corp, Austin, Tex.) and Mesoscale Discovery System. Since the protein and miRNAs data were combined, the number of candidate explanatory variables exceeds the number of samples. In this situation, the use of the unpenalized methods is not appropriate, thus models were built and performance was evaluated using the penalized logistic regression with LOOV or k-fold cross-validation for the calculation of the prevalidated score as described above. FIG. 5 provides the AUC distribution for models based on both miRNAs and proteins. The AUC is statistically equivalent with the ones obtained for miRNAs only, but two miRNAs were consistently selected in the models (see Table 15). FIG. 6 shows the distribution of miRNAs and protein correlations, while FIG. 7 presents the distribution of miRNAs only. The two perpendicular lines in FIG. 6 represent the highest and lowest correlation between protein and miRNAs. Without wishing to be bound by any particular theory, these correlations may correspond to regulatory influences that are not currently investigated. Comparison of these two figures indicates that the proteins produce a higher number of positive correlations in this data set.

TABLE 15

| miR | Counts |
|---|---|
| miR.378 | 50 |
| miR.26a | 50 |
| MPO | 50 |
| SP.SELECTIN | 50 |
| VEGF | 50 |
| EOTAXIN | 48 |
| M.HGF | 44 |
| miR.92a | 32 |
| RESISTIN | 29 |
| miR.125a.5p | 25 |
| M.IL.16 | 18 |
| I.TAC | 17 |

### Example 5

### Survival Analysis Using miRNA Biomarkers

In this study, the levels of the miRNA describe the risk of an event (here MI) occurring over time. Univariate and multivariate classification and survival analyses of 112 candidate miRNA markers were performed. Classification results were obtained based on the methodologies described in Examples 2 and 3. Survival analysis was performed using a Cox proportional hazard regression approach. The response variables for the later analysis included the time when an event took place or the time to the end of the study and an index indicating if the time corresponds to an event or the end of the study (censoring). For the 52 samples described in Example 2, the time of event or end of follow-up time was known. For the 26 subjects that had an event before the end of the study, the indicator variable for an event was set to 1 and for the 26 subjects without an event within the duration of the study the indicator variable was set to 0. Explanatory variables included in the analysis were: a) the protein levels alone, b) the miRNA levels alone and c) either the miRNA and/or protein levels. Model fitting was accomplished using both penalized and unpenalized versions of the Cox proportional hazard model. The L1-penalty (Lasso) was used whenever the penalized version of the model was applied. The variable selection for each model was performed using the same approaches described in Example 1, i.e., using a) the Bayesian information criterion with forward selection for the unpenalized version of the models and b) a cross-validation based selection of the optimum penalty for the penalized approach. In order to evaluate the performance of these models in an objective way, the calculation of a prevalidated score obtained in a manner similar to the one described in Example 1 was employed.

In the first analysis (classification), survival time was ignored and all cases were treated the same, regardless of time-to-event. Table 16 shows the results for the univariate classification analysis. The markers in this table have been ordered by the predicted AUC. Table 18 shows the selection frequency of miRNAs in multivariate classification models. Multiple logistic regression models were built during the prevalidation process on training sets obtained through a LOOV approach, providing a score for the left-out-sample. The model size was determined by the use of the Bayesian Information Criterion. The average classification performance was based on the vector of prevalidated classification scores and was equal to 0.7.

TABLE 16

| | Estimate | Std. Error | z value | Pr(>\|z\|) | AUC |
|---|---|---|---|---|---|
| hsa.miR.378 | −1.40 | 0.42 | −3.33 | 0.00 | 0.84 |
| hsa.miR.1974 | 0.68 | 0.30 | 2.29 | 0.02 | 0.76 |
| hsa.miR.26a | 0.74 | 0.28 | 2.61 | 0.01 | 0.76 |
| hsa.miR.30b | 0.95 | 0.35 | 2.75 | 0.01 | 0.74 |
| hsa.miR.29c | −0.71 | 0.30 | −2.34 | 0.02 | 0.74 |
| hsa.miR.34a | −0.62 | 0.29 | −2.11 | 0.03 | 0.73 |
| hsa.miR.30c | 0.71 | 0.31 | 2.28 | 0.02 | 0.72 |
| hsa.miR.221 | 0.86 | 0.33 | 2.63 | 0.01 | 0.72 |
| hsa.miR.192 | −0.87 | 0.33 | −2.60 | 0.01 | 0.72 |
| hsa.miR.122 | −0.76 | 0.30 | −2.51 | 0.01 | 0.71 |
| hsa.miR.19a | −0.54 | 0.29 | −1.86 | 0.06 | 0.71 |
| hsa.let.7a | 0.67 | 0.31 | 2.15 | 0.03 | 0.71 |
| hsa.miR.21 | −0.77 | 0.33 | −2.34 | 0.02 | 0.7 |
| hsa.miR.497 | −0.78 | 0.32 | −2.45 | 0.01 | 0.7 |
| hsa.miR.19b | −0.52 | 0.29 | −1.79 | 0.07 | 0.7 |
| hsa.miR.148a | −0.69 | 0.30 | −2.29 | 0.02 | 0.7 |
| hsa.miR.15b. | −0.53 | 0.27 | −1.94 | 0.05 | 0.69 |
| hsa.miR.331.3p | 0.65 | 0.30 | 2.19 | 0.03 | 0.69 |
| hsa.miR.24 | 0.68 | 0.30 | 2.30 | 0.02 | 0.69 |
| hsa.miR.142.5p | 0.68 | 0.35 | 1.95 | 0.05 | 0.69 |
| hsa.miR.99a | −0.76 | 0.31 | −2.42 | 0.02 | 0.69 |
| hsa.miR.25 | −0.47 | 0.29 | −1.62 | 0.11 | 0.69 |
| hsa.miR.29a | −0.86 | 0.36 | −2.41 | 0.02 | 0.69 |
| hsa.miR.22 | −0.54 | 0.30 | −1.77 | 0.08 | 0.68 |
| hsa.miR.652 | 0.67 | 0.34 | 1.94 | 0.05 | 0.68 |
| hsa.miR.92a | −0.40 | 0.28 | −1.41 | 0.16 | 0.68 |
| hsa.miR.140.3p | −0.48 | 0.29 | −1.63 | 0.10 | 0.68 |

TABLE 17

| miRNA biomarker | Counts |
|---|---|
| hsa.miR.378 | 47 |
| hsa.miR.497 | 47 |
| hsa.miR.24 | 45 |
| hsa.miR.126 | 45 |
| hsa.miR.21 | 42 |
| hsa.miR.15b | 38 |
| hsa.miR.652 | 33 |
| hsa.miR.29a | 26 |
| hsa.miR.99a | 17 |
| hsa.miR.30b | 10 |
| hsa.miR.29c | 6 |
| hsa.miR.331.3p | 4 |
| hsa.miR.19a | 4 |

Table 18 shows the results from the univariate survival analysis. Again, the markers in this table have been ordered by the predicted AUC. Top selected markers were almost identical to those obtained from the classification analysis and overall performance, as measured by time-dependent AUC, was comparable to that obtained from the classification approach. Table 19 shows the selection frequency of the miRNA markers in a multivariate survival analysis using a Cox proportional Hazard regression approach. The expected performance, for miRNA only based models, was estimated using prevalidation (AUC=0.78). Training sets were constructed through a leave-one-out approach and the model size within each fold was determined based on the Bayesian information criterion. The average model size was 8.

TABLE 18

| | coef | exp (coef) | se (coef) | z | Pr (>\|z\|) | AUC |
|---|---|---|---|---|---|---|
| hsa.miR.378 | −0.5 | 0.61 | 0.13 | −3.68 | 0 | 0.82 |
| hsa.miR.1974 | 0.24 | 1.27 | 0.15 | 1.62 | 0.11 | 0.74 |
| hsa.miR.29c | −0.45 | 0.64 | 0.19 | −2.4 | 0.02 | 0.74 |
| hsa.miR.26a | 0.36 | 1.44 | 0.17 | 2.09 | 0.04 | 0.74 |
| hsa.miR.30b | 0.42 | 1.52 | 0.19 | 2.2 | 0.03 | 0.72 |
| hsa.miR.30c | 0.33 | 1.39 | 0.19 | 1.76 | 0.08 | 0.72 |
| hsa.miR.34a | −0.3 | 0.74 | 0.16 | −1.85 | 0.06 | 0.71 |
| hsa.miR.192 | −0.4 | 0.67 | 0.19 | −2.13 | 0.03 | 0.7 |
| hsa.miR.122 | −0.4 | 0.67 | 0.18 | −2.23 | 0.03 | 0.7 |
| hsa.miR.221 | 0.27 | 1.31 | 0.12 | 2.24 | 0.03 | 0.7 |
| hsa.miR.331.3p | 0.41 | 1.51 | 0.18 | 2.33 | 0.02 | 0.7 |
| hsa.miR.497 | −0.44 | 0.65 | 0.18 | −2.44 | 0.01 | 0.7 |
| hsa.miR.652 | 0.41 | 1.51 | 0.19 | 2.12 | 0.03 | 0.7 |
| hsa.miR.21 | −0.48 | 0.62 | 0.21 | −2.3 | 0.02 | 0.7 |
| hsa.let.7a | 0.32 | 1.38 | 0.2 | 1.64 | 0.1 | 0.69 |
| hsa.miR.148a | −0.29 | 0.75 | 0.15 | −1.91 | 0.06 | 0.69 |
| hsa.miR.29a | −0.58 | 0.56 | 0.21 | −2.75 | 0.01 | 0.69 |
| hsa.miR.19a | −0.26 | 0.77 | 0.18 | −1.47 | 0.14 | 0.68 |
| hsa.miR.19b | −0.19 | 0.83 | 0.17 | −1.09 | 0.28 | 0.68 |
| hsa.miR.15b. | −0.34 | 0.71 | 0.17 | −2.01 | 0.04 | 0.68 |

TABLE 19

| miRNA biomarker | Counts |
|---|---|
| hsa.miR.21 | 47 |
| hsa.miR.378 | 47 |
| hsa.miR.652 | 47 |
| hsa.miR.497 | 47 |
| hsa.miR.15b | 47 |
| hsa.miR.99a | 41 |
| hsa.miR.22 | 24 |
| hsa.miR.126 | 13 |
| hsa.miR.29a | 7 |
| hsa.let.7b | 5 |
| hsa.miR.502.3p | 5 |

### Example 6

### Expanded miRNA Screening

In order to further investigate the ability of miRNA biomarkers to distinguish case versus control, RNA extracts previously obtained from the fifty-two serum samples from Example 2, were screened for the presence of 720 miRNA target sequences shown in Table 1, using Exiqon's mercury LNA™ Universal RT microRNA PCR array technology platform, currently updated to miRBASE 13.

A number of analyses were combined to provide an overall significance of each miRNA biomarker. Univariate classification and survival analyses provided AUC values for each individual miRNA target which were used to rank each target in order of significance. Multivariate analysis was also conducted to generate 47 multivariate models. miRNA targets were ranked by the number of models for which they were selected. A t-test analysis (1-tailed) was also conducted comparing Cp values measured for each miRNA target in the case and control populations. Lastly, a quartile analysis was conducted for the data set. For each miRNA target, all samples (combined case and control populations) were ranked according to Cp value (low to high). The ranked population was then divided into four quartiles, each containing 25% of the total population. The number of case and control subjects in each quartile was then recorded. If greater than 65% or less than 35% of the total number of 26 cases were ranked in the “low” quartile, then that miRNA target was considered significant.

Based on the analysis of the expanded set of 720 miRNA biomarkers, a final overall rank score was assigned, which describes the generation of an overall significance score by which the entire set of miRNA targets was ranked. Table 20 shows the top 50 scoring miRNAs.

TABLE 20

| Biomarker | SCORE | Rank |
|---|---|---|
| miR-378 | 437 | 1 |
| miR-497 | 411 | 2 |
| miR-21 | 392 | 3 |
| miR-15b | 359 | 4 |
| miR-99a | 357 | 5 |
| miR-652 | 356 | 6 |
| miR-30b | 345 | 7 |
| miR-26a | 335 | 8 |
| miR-29a | 329 | 9 |
| miR-1974 | 327 | 10 |
| miR-30c | 325 | 11 |
| miR-122 | 322 | 12 |
| miR-29c | 321 | 13 |
| miR-192 | 321 | 14 |
| miR-34a | 319 | 15 |
| miR-24 | 318 | 16 |
| miR-221 | 317 | 17 |
| miR-126 | 314 | 18 |
| miR-331-3p | 307 | 19 |
| let-7a | 299 | 20 |
| miR-148a | 296 | 21 |
| let-7g | 288 | 22 |
| miR-19a | 287 | 23 |
| miR-142-5p | 284 | 24 |
| miR-22 | 283 | 25 |
| miR-19b | 272 | 26 |
| miR-151-5p | 262 | 27 |
| miR-215 | 261 | 28 |
| miR-25 | 258 | 29 |
| let-7f | 255 | 30 |
| miR-10b | 252 | 31 |
| miR-423-3p | 251 | 32 |
| miR-502-3p | 246 | 33 |
| miR-140.3p | 238 | 34 |
| miR-92a | 235 | 35 |
| miR-660 | 233 | 36 |
| miR-142-3p | 229 | 37 |
| miR-130a | 218 | 38 |
| miR-185 | 217 | 39 |
| let-7c | 215 | 40 |
| miR-18a | 210 | 41 |
| miR-365 | 203 | 42 |
| miR-26b | 194 | 43 |
| miR-125b | 178 | 44 |
| miR-297 | 171 | 45 |
| miR-146a | 151 | 46 |
| miR-99b | 104 | 47 |
| miR-424 | 76 | 48 |
| miR-93 | 60 | 49 |
| let-7b | 14 | 50 |

### Example 7

### Protein Biomarker-Based Cardiovascular Risk Score Development

The development of a cardiovascular risk score was based on a sample of 1123 individuals from the PMRP (Personalized Medicine, 2(1): 49-79 (2005)). The set was selected based on a case-cohort design. Subjects from the PMRP cohort were considered “cases” if they were from 40-80 years old at the time of baseline blood draw and if they had an incident MI or had been hospitalized for unstable angina (UA) during the 5 years of follow-up. There were 385 total cases (164 subjects with initial MI, and 221 subjects with UA) and 838 controls. The available data included 59 (47 unique) protein biomarkers measured for each individual and 107 clinical characteristics including demographic (age, gender, race, diabetes status, family history of MI, smoking, etc.) and laboratory measurements (total cholesterol, HDL, LDL, etc.) and medication use (statin, antihypertensive medication, hypoglycemic medication, etc.).

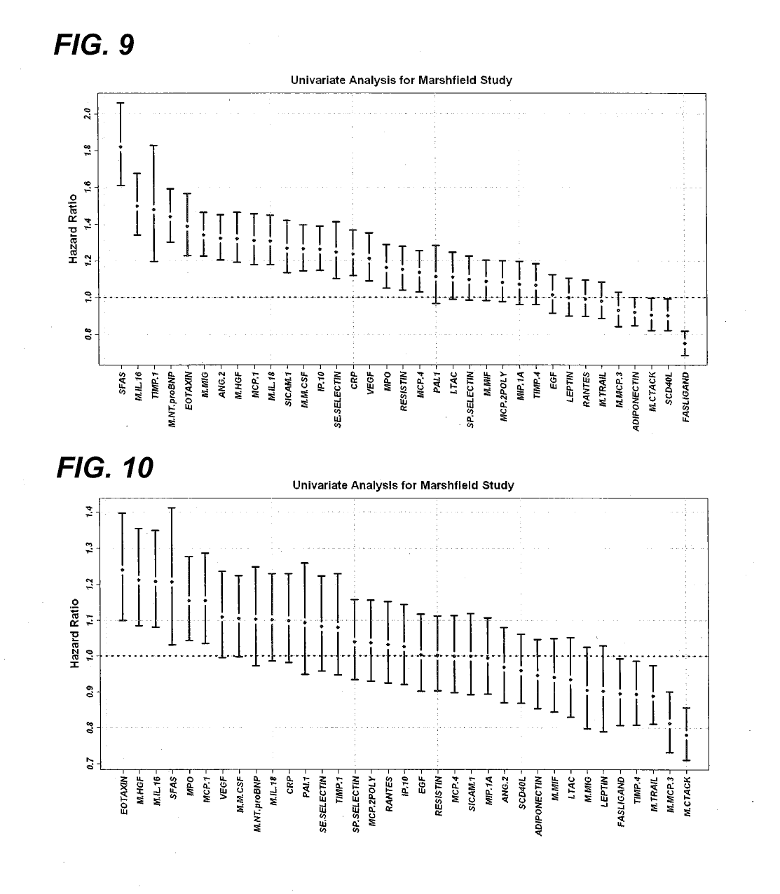

Univariate Analysis. The association of each biomarker with patient outcome was evaluated using a Cox proportional hazard regression and time dependent area under the curve (AUC) using the Kaplan-Meier method of Heagerty et al., (Survival Model Predictive Accuracy and ROC Curves Biometrics, 61:92-105 (2005)). In order to present the hazard ratio (HR) across all protein biomarkers with different concentration ranges on a common scale, the values for all subjects were normalized by subtracting the mean value of the controls' concentration divided by the standard deviation of the controls after log-transforming the data. The hazard ratios were thus expressed per one standard deviation unit. FIG. 9 shows the unadjusted hazard ratio and standard error for the 35 biomarkers that were used as candidates for developing multivariate models of risk. Twenty-two of the biomarkers have an HR that is statistically significant.

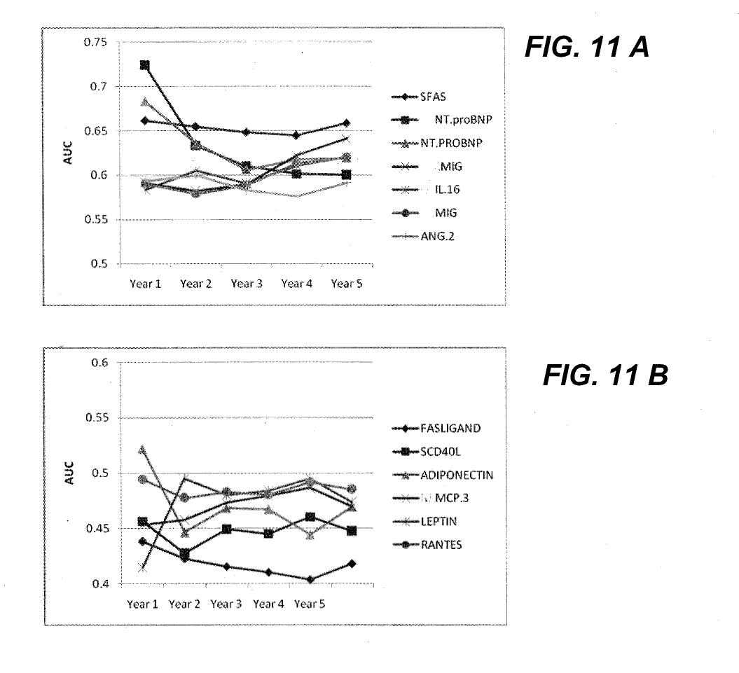

The same analysis was repeated while adjusting each of the biomarkers for the following traditional risk factors (TRFs): age, sex, systolic BP, diastolic BP, cholesterol, HDL, hypertension, use of hypertension drug, hyperlipidemia, diabetes, smoking (FIG. 10). After adjustment, only 11 of the biomarkers maintained statistical significance, which is not surprising since the TRFs chosen were known to be associated with cardiovascular disease. FIGS. 11 A and B show the markers with the highest time-dependent AUC and the corresponding values for up to 5 years of follow-up. The AUC for all of the markers remained constant with time with the exception of the two versions of the NT-proBNP assay, which showed a decrease with time.

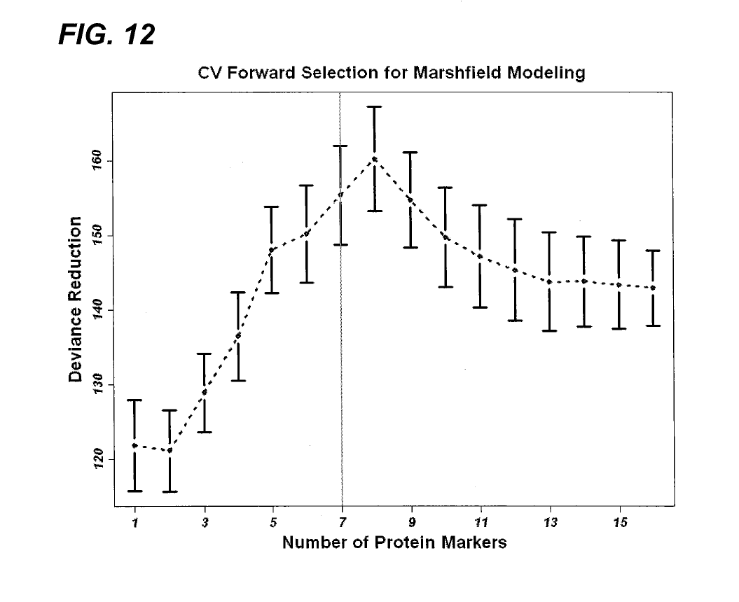

Multivariate analysis: development of prognostic score for MI and/or UA. The development of a prognostic score was based on the inclusion of TRFs as well as protein biomarkers. Given the known association of age, gender, diabetes, and family history with cardiovascular events, these four parameters were included in the model. The inclusion of these 4 parameters was confirmed by running a number of forward marker selection algorithms. All of the algorithms selected the four variables in the final multivariate algorithms. The determination of the optimum model size was based on the use of the following criteria: (a) Akaike information criterion, (b) Bayesian information criterion, (c) Drop-in-deviance criterion. The first 2 are known in-sample error estimators and the third utilizes a cross-validation loop to estimate the goodness-of-fit. In all three cases, the model size was selected for the model that best fit the data, avoiding overfitting. A characteristic drop-in-deviance curve for model selection (a plot of the absolute value of the quantity) is shown in FIG. 12. The size of the model was selected based on using the 1 standard error rule, i.e., the maximum of the curve was identified and then a line was drawn from the 1 standard error point below the maximum. The optimum number of protein biomarkers was selected as the smallest number that its corresponding average absolute deviance value exceeded the aforementioned line. That number corresponded to 7 protein biomarkers, i.e., the optimum risk score was therefore composed of 4 TRFs and 7 protein biomarkers (FIG. 12). All three methods selected between 5 and 7 biomarkers as the optimum number of biomarkers in the model. The smaller set of biomarkers was always a subset of the larger set. Table 21 shows the frequency and ranking of the selected biomarkers after age, gender, diabetes, and family history of MI have been inserted into the model. These counts and rankings were obtained from the different models that were built during the cross-validation process; one model is, built for every training fold, the size of which is selected by one of the model selection methods mentioned above. The cross-validation process was repeated in order to average over the variability introduced by the membership assignment of each subject.

TABLE 21

| | Counts | | | |
|---|---|---|---|---|
| Biomarker | (out of 20) | Average Rank | Min Rank | Max Rank |
| EOTAXIN | 20 | 3.7 | 2 | 7 |
| IL.16 | 20 | 1.05 | 1 | 2 |
| MCP.3 | 20 | 4.4 | 2 | 7 |
| CTACK | 17 | 2.9 | 2 | 5 |
| ADIPONECTIN | 16 | 5.4 | 2 | 9 |
| HGF | 12 | 5.1 | 1 | 9 |
| FASLIGAND | 10 | 6.0 | 2 | 8 |
| SFAS | 10 | 6.6 | 5 | 8 |
| IL.18 | 9 | 7.7 | 4 | 12 |
| TIMP.4 | 7 | 7.0 | 3 | 11 |
| TIMP.1 | 5 | 8.4 | 5 | 12 |
| CRP | 4 | 6.3 | 4 | 9 |
| HGF | 4 | 7.5 | 3 | 11 |
| VEGF | 3 | 7.7 | 7 | 8 |
| EGF | 1 | 6.0 | 6 | 6 |

Table 21 shows the frequency selection, average, minimum and maximum rank of each biomarker over 4 repeats of a 5-fold prevalidation (a form of cross-validation) process. The 4 TRFs were included in each of the models.

Using the optimum model size predicted by the drop-in-deviance approach, a Cox proportional hazard model was fit to all available data in order to obtain a model that could be used for validation on a different population. This final protein-based model contained the following protein biomarkers in the order selected: IL-16, eotaxin, fasligand, CTACK, MCP-3, HGF, and sFas.

### Example 8

### Comparison of Protein Model to Other Standard Predictive Models

The transportability of the disclosed model for predicting risk of cardiovascular event (ie, MI or UA) was assessed in a second multi-ethnic cohort selected from the U.S. population, ages 45-84 years old (Multi Ethnic Study of AtheroSclerosis Cohort) [Bild D E, Bluemke D A, Burke G L, Detrano R, Diez Roux A V, Folsom A R, Greenland P, Jacob D R, Jr., Kronmal R, Liu K, Nelson J C, O'Leary D, Saad M F, Shea S, Szklo M, Tracy R P. Multi-ethnic study of atherosclerosis: objectives and design. Am J Epidemiol. 2002; 156(9):871-881.

In order to establish the expected performance of the model on a different sample similar to the one used for development, the method of prevalidation was used again, before applying the model to the second population. Two performance metrics were used: the Net Reclassification Index (NRI) and the Clinical Net Reclassification Index (CNRI). The definition of the net reclassification index is given by the following equation:

$$NRI = \frac{Cases Up - Cases Down}{No . of cases in risk category} - \frac{Controls Up - Controls Down}{No . of controls in risk category}$$

The equation measures the improvement for the cases and controls separately in terms of a percent and combines the results into a single number. A positive percentile for the cases and a negative for the controls represents improvement in performance introduced by the disclosed model. The risk category is defined by establishing appropriate thresholds for the risk scores predicted by the existing and disclosed models. The CNRI is defined in the same way but applies to a subset of the population that can gain from an improved method of identifying the true risk within the group. For cardiovascular disease, application of the NRI metric in the intermediate risk population, as defined by the Franimgham score for example, satisfies this criterion. The calculated value represents the CNRI performance for the intermediate risk category.

Traditionally, the intermediate risk category, as calculated by the Framingham score for 10 year risk, has been defined as those individuals with risk score between 10% and 20%. The results presented here are based on the following cutoffs for defining the intermediate risk category: <3.5%, >7.5%. The use of these lower cutoffs is justified because: a) the disclosed model focuses on a time horizon of 5 years, and b) the event rate in the current population is lower than the one observed when the Framingham score was developed.

The reclassification comparison required the calculation of an absolute risk, from each model, for a given subject. The calculation of an absolute risk for each individual using a Cox Proportional Hazard (Cox PH) model required the calculation of the relative risk for this individual based on their characteristics and the estimation of a baseline hazard. The Cox PH model is designed to predict the relative risk but does not require specification of the hazard function. To produce absolute risk estimates from a Cox PH model, we needed the absolute risk for any individual, or for an “average” individual; then using the risk estimates relative to this individual or the average, the absolute risk for any individual was computed. The average is a hypothetical individual with the population average value for each predictor. Given that the true baseline hazard for the population and the corresponding “average” person are not known (because the correct model for the calculation of the risk of a cardiovascular event is unknown), an estimate needed to be provided. The R language [R: A Language and Environment for Statistical Computing, R Development Core Team, R Foundation for Statistical Computing, Vienna, Austria, 2010] survfit function was used to calculate the baseline hazard for the average individual. The survfit function uses weights for the calculation: each member of the population receives a weight depending on their estimated risk score relative to the average, and then a weighted hazard estimate is used for the baseline hazard. The estimation of a baseline hazard depends on the model used and hence also upon the predicted relative risk. In order to make fair comparisons of the reclassification performance of the disclosed model vs. the FRS and TRF-based models, an appropriate baseline hazard estimate was needed that did not unduly favor any one model. Described below is the preferred approach for the calculation of the baseline hazard that used a risk score that is the average score from the two models being compared. In addition, the survfit function implemented two alternative estimators: Kaplan-Meier and Aalen. Both estimators were tested and the difference observed was negligible. In order to extend our conclusions to the population, the baseline survivor function was evaluated at the population mean of the covariates using the case-cohort weights of the study.

The selection of a baseline hazard estimate for comparing two models in terms of absolute risk score is a difficult problem, and one not addressed in the literature. Because the true baseline hazard for the population is unknown, the use of a different estimate by each model can have a significant effect on the results of the comparison. To investigate the effect of the baseline hazard estimate, all calculations were performed using two different methods: 1.) the absolute risk score for each model based on the individual baseline survivor estimate using the linear predictor scores calculated by each model; and 2.) the absolute risk score based on a common baseline survivor estimate obtained by calculating the average linear predictor from the two scores, centered at the population mean.

Tables 22, 23, and 24 present the NRI and CNRI expected performance of the pre-validated models containing biomarkers against three alternative models: 1.) the Framingham risk score (“FRS”); 2.) a model fitted on the Marshfield data using 4 TRFs (“4-TRF”; age, gender, diabetes, and family history of MI) as covariates; and 3.) an alternate model fitted on the Marshfield data using 9 TRFs (“9-TRF”; age, gender, diabetes, family history of MI, smoking, total cholesterol, HDL, hypertension medication, and systolic pressure) as covariates.

Overall, the models that included protein biomarkers provided a better reclassification over the FRS or TRF-based models in both the 3.5-7.5% and 3.5-10% ranges of 5 year risk for a cardiovascular event. Table 22 shows the expected reclassification performance of the disclosed model score against the calibrated FRS score based on pre-validation (Marshfield data set). Tables 23 and 24 show the expected reclassification score against the 4-TRF and 9-TRF model scores, respectively, based on pre-validation (Marshfield data set).

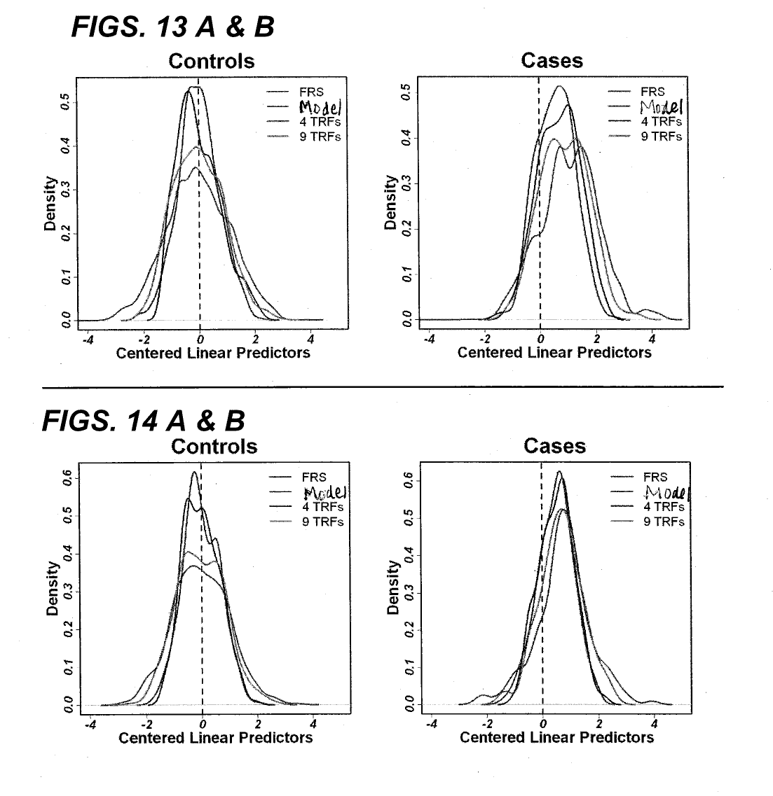

The overall reclassification in terms of both NRI and CNRI were comparable using either of the two methods for calculating the baseline survivor function. There was, however, a difference in the reclassification balance of cases and controls that make up the total NRI or CNRI between the two methods. The common baseline survivor function method did provide a more balanced reclassification. This result was consistent with the results obtained for the relative risk prediction of the models. FIGS. 13 A-B present this comparison in terms of the kernel density estimate of the linear scores of the FRS, the disclosed model (obtained from multiple repeats of the pre-validation approach), 4-TRF, and the 9-TRF models. The disclosed model score provided a higher relative risk for cases than any model. The distribution for the controls was also wider for the disclosed model score indicating a balance of up and down risked controls compared to the other scores. These results provided a strong indication that the disclosed model score correctly up-classified cases with respect to the other scores.

The common baseline survivor function method (using the average score) was also consistent with many statistical approaches that use a voting scheme (i.e. weighted averaging) for improving prediction accuracy.

TABLE 22

| | | Baseline | | | | | | |
|---|---|---|---|---|---|---|---|---|
| | | Hazard | | | | | | |
| | Range | calculation | NRI (sd) | NRI_case | NRI_ctrl | CNRI (sd) | CNRI_case | CNRI_ctrl |
| FRS | 3.5-7.5% | Individual | 10.34% [1.85%] | 6.1% [2.11%] | −4.24% [0.66%] | 44.52% [4.5%] | 2.95% [4.8%] | −41.56% [1.83%] |
| | | Average | 15.18% [2.26%] | 23.23% [1.45%] | 8.05% [1.42%] | 48.51% [5.42%] | 27.33% [3.31%] | −21.19% [4.05%] |
| | 3.5-10.0% | Individual | 9.39% [2.1%] | 5.41% [1.46%] | −3.98% [0.8%] | 42.19% [4.92%] | 1.74% [3.41%] | −40.45% [2.76%] |
| | | Average | 15.94% [1.2%] | 24.23% [1.69%] | 8.28% [0.88%] | 44.07% [2.05%] | 21.31% [3.06%] | −22.76% [2.59%] |

Expected Reclassification performance of Aviir score against the calibrated Framingham score based on pre-validation (Marshfield data set)

TABLE 23

| | | Baseline | | | | | | |
|---|---|---|---|---|---|---|---|---|
| | | Hazard | | | | | | |
| | Range | calculation | NRI (sd) | NRI_case | NRI_ctrl | CNRI (sd) | CNRI_case | CNRI_ctrl |
| 4-TRF | 3.5-7.5% | Individual | 6.92% [1.39%] | 5.3% [1.71%] | −1.62% [0.69%] | 33.42% [3.58%] | 11.38% [3.99%] | −22.04% [3.12%] |
| | | Average | 13.24% [2.2%] | 24.39% [1.86%] | 11.15% [0.72%] | 31.52% [4.72%] | 34.64% [3.71%] | 3.12% [3.04%] |
| | 3.5-10.0% | Individual | 9.56% [2.4%] | 7.32% [2.04%] | −2.24% [0.76%] | 29.83% [3.84%]″ | 6.61% [2.79%] | −23.22% [2.31%] |
| | | Average | 15.23% [1.86%] | 25.91% [1.76%] | 10.68% [0.48%] | 31.86% [3.76%] | 29.07% [3.27%] | −2.78% [1.7%] |

Expected Reclassification performance of Aviir score against the 4-TRF model score based on pre-validation (Marshfield data set)

TABLE 24

| | | Baseline | | | | | | |
|---|---|---|---|---|---|---|---|---|
| | | Hazard | | | | | | |
| | Range | calculation | NRI (sd) | NRI_case | NRI_ctrl | CNRI (sd) | CNRI_case | CNRI_ctrl |
| 9-TRF | 3.5-7.5% | Individual | −0.1% [1.52%] | −1.23% [1.69%] | −1.12% [0.81%] | 29.86% [4.23%] | 4.94% [3.53%] | −24.93% [2.73%] |
| | | Average | 3.95% [1.81%] | 9.78% [1.77%] | 5.83% [0.66%] | 28.77% [3.78%] | 19.95% [3.68%] | −8.82% [1.86%] |
| | 3.5-10.0% | Individual | 1.9% [1.7%] | 0.73% [1.71%] | −1.17% [0.73%] | 28.25% [3.8%] | 1.95% [2.67%] | −26.3% [2.46%] |
| | | Average | 7.19% [1.84%] | 12.65% [1.54%] | 5.46% [0.76%] | 28.35% [3.83%] | 16.32% [2.94%] | −12.03% [2.05%] |

Expected Reclassification performance of Aviir score against the 9-TRF model score based on pre-validation (Marshfield data set)

### Example 9

### Transportability of Disclosed Model to a Second Population

The question of transportability of a prognostic model across multiple populations provides the ultimate test for the usefulness of the prediction model. A model's statistical and clinical validity are equally important facets of a model's transportability. A three-step validation approach has been proposed for a new test: 1) internal validation, 2) temporal validation, and 3) external validation. The completion of the first step by using pre-validation approach (a form of cross-validation) to validate the modeling methods was described above. The second step requires the testing of the algorithm on a different patient set from the same population or clinical center. Given that there is only a short period of time (about 2 years) between the time that the last event took place within the Marshfield study and the current time, the number of subsequent events was too small for validation within the same population. Therefore, the external validation step was conducted by testing the disclosed protein model on the MESA sample set as a demonstration of the disclosed protein model's transportability.

To evaluate the disclosed model's performance on the MESA cohort, 824 samples (222 cases and 602 controls) were assayed using the panel of protein biomarkers described in Example 7 (IL-16, eotaxin, fas ligand, CTACK, MCP-3, HGF, and sFas).

The Marshfield-trained model was used to predict a score for each subject of the MESA sample with marker selection and model fitting performed on the Marshfield population without any knowledge or input from the MESA results.

The calculations of the absolute risk scores for all models were based on the approaches described above. Due to some missing values for some of the risk factors and the biomarkers, the cohort weights were modified for the combination of status and gender in each of the comparisons. The calculations of the reclassifications also accounted for the same modified weights, because the reclassification of a female and a male case or control does not carry the same weight. This was done in an attempt to properly extend the results to the total population assuming that the missing values were missing at random.

Tables 25 and 26 present the comparison between the disclosed model vs. the 3 other models in terms of NRI and CNRI presented earlier, as well comparison against the Reynolds score [Ridker P M, Buring J E, Rifai N, et al. Development and validation of improved algorithms for the assessment of global cardiovascular risk in women: the Reynolds Risk Score JAMA 2007; 297:611-619]. The comparisons were consistent with the predicted performance from the Marshfield set. The disclosed model provided better clinical net reclassification over any other transported model presented here. The method using the average of the scores for estimating the baseline survivor function also provided a better balance in reclassification between cases and controls, when compared to the method using the individual estimates. This was again consistent with the relative risk predictions for these models on the MESA samples (FIGS. 14 A and B). These results clearly support the clinical usefulness and transportability of the disclosed model for the low intermediate/intermediate risk populations in the MESA set. The predictive ability of the model in the non-diabetic population is shown in Table 27 in terms of NRI and CNRI. For the later the intermediate range of risk is set to the 3.5 to 7.5% interval based on the reference model. All subjects with diagnosed diabetes at baseline have been excluded from the comparison. The results again show the clinical utility of the model in the intermediate risk category for non-diabetic subjects.

TABLE 25

| | Baseline | | | | | | | | |
|---|---|---|---|---|---|---|---|---|---|
| | Hazard | | | | | | | | |
| | Calculation | NRI | NRI pval | NRI Case | NRI Ctrl | CNRI | CNRI pval | CNRI Case | CNRI Ctrl |
| FRS | individual | 1.906% | 0.3425 | −3.568% | −5.474% | 31.931% | 0.0000 | 2.076% | −29.855% |
| | average | 2.706% | 0.2895 | 7.130% | 4.424% | 30.254% | 0.0000 | 12.311% | −17.943% |
| 4-TRFs | individual | 6.071% | 0.0650 | −0.611% | −6.682% | 23.566% | 0.0000 | 2.198% | −21.368% |
| | average | 12.266% | 0.0025 | 19.505% | 7.238% | 23.932% | 0.0000 | 20.426% | −3.505% |
| 9-TRFs | individual | −0.289% | 0.5269 | −3.324% | −3.035% | 20.211% | 0.0002 | 2.407% | −17.804% |
| | average | 2.257% | 0.3033 | 4.479% | 2.222% | 18.404% | 0.0012 | 8.400% | −10.004% |
| Reynolds | individual | −5.045% | 0.8436 | −6.102% | −1.057% | 26.697% | 0.0001 | 9.231% | −17.466% |
| | average | −8.490% | 0.9606 | −15.562% | −7.072% | 25.202% | 0.0003 | 3.380% | −21.822% |

NRI and CNRI results for the MESA data set comparing the Aviir score against FRS, 4-TRF, 9-TRF and Reynolds score models. The CNRI is based on a baseline range of risk of 3.5-10% of the reference model. Subjects with missing biomarker data have been excluded from the comparison.

TABLE 26

| | Baseline | | | | | | | | |
|---|---|---|---|---|---|---|---|---|---|
| | Hazard | | | | | | | | |
| | Calculation | NRI | NRI pval | NRI Case | NRI Ctrl | CNRI | CNRI pval | CNRI Case | CNRI Ctrl |
| FRS-individ | individual | 0.247% | 0.4805 | −9.878% | −10.125% | 46.363% | 0.0000 | 12.836% | −33.527% |
| FRS-average | average | 0.657% | 0.4477 | 4.875% | 4.218% | 39.596% | 0.0000 | 24.328% | −15.268% |
| TRF4-individ | individual | 2.703% | 0.2660 | −7.622% | −10.325% | 30.501% | 0.0000 | 4.666% | −25.834% |
| TRF4-average | average | 2.902% | 0.2520 | 10.940% | 8.038% | anal | 0.0269 | 19.772% | 4.296% |
| TRFext-individ | individual | −3.249% | 0.7582 | −9.115% | −5.866% | 32.157% | 0.0001 | 11.602% | −20.556% |
| TRFext-average | average | −1.072% | 0.5895 | 2.162% | 3.234% | 27.144% | 0.0017 | 23.674% | −3.470% |
| Reynold-individ | individual | −3.951% | 0.7919 | −3.172% | 0.779% | 33.933% | 0.0008 | 19.294% | −14.639% |
| Reynold-average | average | −6.377% | 0.9229 | −11.151% | −4.774% | 22.063% | 0.0257 | 2.718% | −19.345% |

NRI and CNRI results for the MESA data set comparing the Aviir score against FRS, 4-TRF, 9-TRF and Reynolds score models. The CNRI is based on a baseline range of risk of 3.5-7.5% of the reference model. Subjects with missing biomarker data have been excluded from the comparison.

TABLE 27

| | | Baseline | | | | | | | | |
|---|---|---|---|---|---|---|---|---|---|---|
| | | Hazard | | | | | | | | |
| | Range | Calculation | NRI | NRI p-val | NRI_case | NRI_ctrl | CNRI | CNRI p-val | CNRI_case | CNRI_ctrl |
| FRS | 3.5-7.5% | Individual | 0.42% | 0.472 | −1.23% | −1.65% | 38.42% | 0.000 | 13.94% | −24.47% |
| | | Average | 4.64% | 0.211 | 9.84% | 5.21% | 42.31% | 0.000 | 23.28% | −19.02% |
| 4-TRFs | 3.5-7.5% | Individual | 2.31% | 0.324 | −1.20% | −3.51% | 23.48% | 0.006 | 5.06% | −18.42% |
| | | Average | 9.44% | 0.034 | 20.11% | 10.67% | 29.63% | 0.001 | 34.91% | 5.28% |
| 9-TRFs | 3.5-7.5% | Individual | 3.69% | 0.256 | 3.24% | −0.45% | 30.17% | 0.001 | 17.81% | −12.36% |
| | | Average | 6.78% | 0.111 | 12.03% | 5.25% | 28.88% | 0.003 | 26.59% | −2.29% |

NRI and CNRI results for the MESA data set comparing the Aviir score against FRS, 4-TRF and 9-TRF models for non-diabetic individuals in the MESA set. The CNRI is based on a baseline range of risk of 3.5-7.5% of the reference model. Subjects with missing biomarker data have been excluded from the comparison.

### Example 10

### Hybrid Biomarker Prognostic/Diagnostic Model

In addition to the protein biomarker/TRF, miRNAs can be measured in a human fluid, such as blood, and used to predict future cardiovascular events in a subject.

The prognostic power of a hybrid miRNA/protein biomarker set is determined by building a hybrid prognostic model with covariates selected from the miRNA set presented in Table 28 and the disclosed protein biomarker model (see Examples 7-9) as single score, using a case-cohort study design. The cohort contains all of the cases that developed MI within the time frame of interest (n=200) and 200 controls. In order to efficiently utilize the smaller cohort, the TRFs and protein predictors are treated in terms of a single calculated score (single variable), unless univariate association of the miRNA biomarkers is stronger than that observed for the protein biomarkers or TRFs. In the latter case, multivariate models are built based on the use of penalized regression methods selecting variables from all available biomarkers (TRFs, protein biomarkers, miRNAs). In the former case, the score calculation is performed using the coefficients previously estimated on the larger cohort, described above. Cross-validation and penalized regression techniques are used to select the model size and miRNA markers for three types of models: a) miRNA-only model; b) a TRF+miRNA-based model; and c) a TRF+protein+miRNA biomarker-based model. The expected performance of the fitted models is evaluated based on the time-dependent AUC, NRI, and CNRI characteristics of the hybrid models vs. the FRS as well as the previously disclosed TRF+protein-based model (see Examples 8-9)

TABLE 28
miRNAs

| | |
|---|---|
| miR-378 | miR-19b |
| miR-497 | miR-151-5p |
| miR-21 | miR-215 |
| miR-15b | miR-25 |
| miR-99a | let-7f |
| miR-652 | miR-10b |
| miR-30b | miR-423-3p |
| miR-26a | miR-502-3p |
| miR-29a | miR-140.3p |
| miR-1974 | miR-92a |
| miR-30c | miR-660 |
| miR-122 | miR-142-3p |
| miR-29c | miR-130a |
| miR-192 | miR-185 |
| miR-34a | let-7c |
| miR-24 | miR-18a |
| miR-221 | miR-365 |
| miR-126 | miR-26b |
| miR-331-3p | miR-125b |
| let-7a | miR-297 |
| miR-148a | miR-146a |
| let-7g | miR-99b |
| miR-19a | miR-424 |
| miR-142-5p | miR-93 |
| miR-22 | let-7b |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

1. A method for assessing the cardiovascular health of a human comprising:
a) obtaining a biological sample from a human;
b) determining levels of at least 2 miRNA markers selected from miRNAs listed in Table 20 in the biological sample;
c) obtaining a dataset comprised of the levels of each miRNA marker;
d) inputting the data into an analytical classification process that uses the data to classify the biological sample, wherein the classification is selected from the group consisting of an atherosclerotic cardiovascular disease classification, a healthy classification, a medication exposure classification, a no medication exposure classification; and
e) determining a treatment regimen for the human based on the classification in step (d);
wherein the cardiovascular health of the human is assessed.

2. The method of claim 1, wherein the at least 2 miRNA markers are selected from the group consisting of miR-378, miR-497, miR-21, miR-15b, miR-99a, miR-29a, miR-24, miR-30b, miR-29c, miR-331.3p, miR-19a, miR-22, miR-126, let-7b, miR-502.3, and miR-652.

3. The method of claim 2, wherein the at least 2 miRNA markers are selected from the group consisting of miR-378, miR-497, miR-21, miR-15b, miR-99a, and miR-652.

4. The method of claim 1, wherein the atherosclerotic cardiovascular disease classification is selected from the group consisting of coronary artery disease, myocardial infarction, and unstable angina.

5. The method of claim 1, further comprising using the classification for determining atherosclerosis diagnosis, atherosclerosis staging, atherosclerosis prognosis, vascular inflammation levels, extent of atherosclerosis progression, monitoring a therapeutic response, predicting a coronary calcium score, distinguishing stable from unstable manifestations of atherosclerotic disease, and a combination thereof.

6. The method of claim 1, wherein the dataset further comprises data for one or more clinical indicia.

7. The method of claim 6, wherein the one or more clinical indicia are selected from the group consisting of age, gender, LDL concentration, HDL concentration, triglyceride concentration, blood pressure, body mass index, CRP concentration, coronary calcium score, waist circumference, tobacco smoking status, previous history of cardiovascular disease, family history of cardiovascular disease, heart rate, fasting insulin concentration, fasting glucose concentration, diabetes status, use of high blood pressure medication, and a combination thereof.

8. The method of claim 7, wherein the clinical indicia selected are age, gender, diabetes, and family history of MI.

9. The method of claim 1, wherein the biological sample comprises blood, serum, plasma, saliva, urine, sweat, breast milk, and a combination thereof.

10. The method of claim 1, further comprising determining levels of at least one protein biomarker in the biological sample.

11. The method of claim 10, wherein the at least one protein biomarker is selected from the group consisting of IL-16, sFas, Fas ligand, MCP-3, HGF, CTACK, EOTAXIN, adiponectin, IL-18, TIMP.4, TIMP.1, CRP, VEGF, and EGF.

12. The method of claim 11, wherein the at least one protein biomarker is selected from the group consisting of IL-16, EOTAXIN, Fas ligand, CTACK, MCP-3, HGF, and sFAS.

13. The method of claim 11, wherein three or more protein biomarker levels are determined.

14. The method of claim 1, wherein the analytical classification process comprises the use of a predictive model.

15. The method of claim 1, wherein the analytical classification process comprises comparing the obtained dataset with a reference dataset.

16. The method of claim 13, wherein the predictive model comprises at least one quality metric of at least 0.68 for classification.

17. The method of claim 15, wherein the quality metric is selected from AUC and accuracy.

18. The method of claim 1, wherein the analytical classification process comprises using one or more selected from the group consisting of a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, and a combination thereof.

19. The method of claim 18, wherein the analytical classification process comprises terms selected to provide a quality metric of at least 0.68.

20. The method of claim 18, wherein the analytical classification process comprises terms selected to provide a quality metric of 0.70.

21. The method of claim 18, wherein the analytical classification process comprises at least one quality metric of at least 0.70 for classification.

22. The method of claim 1, wherein the treatment regimen comprises one or more selected from the group consisting of further testing, pharmacologic intervention, no treatment, and a combination thereof.

23. A method for assessing the cardiovascular health of a human comprising:
a) obtaining a biological sample from a human;
b) determining levels of at least 3 protein markers selected from the group consisting of IL-16, sFas, Fas ligand, MCP-3, HGF, CTACK, EOTAXIN, adiponectin, IL-18, TIMP.4, TIMP.1, CRP, VEGF, and EGF in the biological sample;
c) obtaining a dataset comprised of the levels of each protein marker;
d) inputting the data into an analytical classification process that uses the data to classify the biological sample, wherein the classification is selected from the group consisting of an atherosclerotic cardiovascular disease classification, a healthy classification, a medication exposure classification, a no medication exposure classification; and
e) determining a treatment regimen for the human based on the classification in step (d);
wherein the cardiovascular health of the human is assessed.

24. The method of claim 23, wherein the at least 3 protein markers are selected from the group consisting of IL-16, EOTAXIN, Fas ligand, CTACK, MCP-3, HGF, and sFAS.

25. The method of claim 23, wherein the dataset further comprises data for one or more clinical indicia selected from the group consisting of age, gender, LDL concentration, HDL concentration, triglyceride concentration, blood pressure, body mass index, CRP concentration, coronary calcium score, waist circumference, tobacco smoking status, previous history of cardiovascular disease, family history of cardiovascular disease, heart rate, fasting insulin concentration, fasting glucose concentration, diabetes status, use of high blood pressure medication, and a combination thereof.

26. A method for assessing the cardiovascular health of a human to determine the need for or effectiveness of a treatment regimen comprising:
obtaining a biological sample from a human;
determining levels of at least 2 miRNA markers selected from miRNAs listed in Table 20 in the biological sample;
determining levels of at least 3 protein biomarker selected from the group consisting of IL-16, sFas, Fas ligand, MCP-3, HGF, CTACK, EOTAXIN, adiponectin, IL-18, TIMP.4, TIMP.1, CRP, VEGF, and EGF in the biological sample;
obtaining a dataset comprised of the individual levels of the miRNA markers and the protein biomarkers;
inputting the data into an analytical classification process that uses the data to classify the biological sample, wherein the classification is selected from the group consisting of an atherosclerotic cardiovascular disease classification, a healthy classification, a medication exposure classification, a no medication exposure classification; and
classifying the biological sample according to the output of the classification process and determining a treatment regimen for the human based on the classification.

27. The method of claim 26, wherein the miRNA markers are selected from the group consisting of miR-378, miR-497, miR-21, miR-15b, miR-99a, miR-29a, miR-24, miR-30b, miR-29c, miR-331.3p, miR-19a, miR-22, miR-126, let-7b, miR-502.3, and miR-652.

28. The method of claim 26, wherein the protein biomarkers are selected from the group consisting of IL-16, EOTAXIN, Fas ligand, CTACK, MCP-3, HGF, and sFAS.

29. A kit for assessing the cardiovascular health of a human to determine the need for or effectiveness of a treatment regimen, comprising:
an assay for determining levels of at least 2 miRNA markers selected from miRNAs listed in Table 20 in the biological sample;
instructions for obtaining a dataset comprised of the individual levels of the miRNA markers, inputting the data into an analytical classification process that uses the data to classify the biological sample, wherein the classification is selected from the group consisting of an atherosclerotic cardiovascular disease classification, a healthy classification, a medication exposure classification, a no medication exposure classification; and classifying the biological sample according to the output of the classification process and determining a treatment regimen for the human based on the classification.

30. The kit of claim 29, further comprising an assay for determining levels of at least 3 protein biomarker selected from the group consisting of IL-16, sFas, Fas ligand, MCP-3, HGF, CTACK, EOTAXIN, adiponectin, IL-18, TIMP.4, TIMP.1, CRP, VEGF, and EGF in the biological sample; and instructions for obtaining a dataset comprised of the individual levels of the protein markers, inputting the data of the miRNA and protein markers into an analytical classification process that uses the data to classify the biological sample, wherein the classification is selected from the group consisting of an atherosclerotic cardiovascular disease classification, a healthy classification, a medication exposure classification, a no medication exposure classification; and classifying the biological sample according to the output of the classification process and determining a treatment regimen for the human based on the classification.

31. A method for assessing the risk of a cardiovascular event of a human comprising:
a) obtaining a biological sample from a human;
b) determining levels of at least 2 miRNA markers selected from miRNAs listed in Table 20 in the biological sample;
c) obtaining a dataset comprised of the levels of each miRNA marker;
d) inputting the data into an risk prediction analysis process to determine the risk of a cardiovascular event based on the dataset; and
e) determining a treatment regimen for the human based on the predicted risk of a cardiovascular event in step (d);
wherein the risk of a cardiovascular event of the human is assessed.

32. The method of claim 31, wherein the risk of a cardiovascular event is determined for a period of time selected from the group consisting of about 1 year, about 2 years, about 3 years, about 4 years, and about 5 years from the date the sample is obtained.

33. The method of claim 31, further comprising determining levels of 3 or more protein biomarkers in the biological sample.

34. The method of claim 33, wherein the 3 or more protein biomarkers are selected from the group consisting of IL-16, sFas, Fas ligand, MCP-3, HGF, CTACK, EOTAXIN, adiponectin, IL-18, TIMP.4, TIMP.1, CRP, VEGF, and EGF.

35. The method of claim 34, wherein the three or more protein biomarkers are selected from the group consisting of IL-16, EOTAXIN, Fas ligand, CTACK, MCP-3, HGF, and sFAS.

36. A method for assessing the risk of a cardiovascular event of a human comprising:
a) obtaining a biological sample from a human;
b) determining levels of more protein biomarkers are selected from the group consisting of IL-16, sFas, Fas ligand, MCP-3, HGF, CTACK, EOTAXIN, adiponectin, IL-18, TIMP.4, TIMP.1, CRP, VEGF, and EGF in the sample;
c) obtaining a dataset comprised of the levels of each protein biomarker;
d) inputting the data into an risk prediction analysis process to determine the risk of a cardiovascular event based on the dataset; and
e) determining a treatment regimen for the human based on the predicted risk of a cardiovascular event in step (d);
wherein the risk of a cardiovascular event of the human is assessed.

37. The method of claim 36, wherein the risk of a cardiovascular event is determined for a period of time selected from the group consisting of about 1 year, about 2 years, about 3 years, about 4 years, and about 5 years from the date the sample is obtained.
